(19) United States
(12) Patent Application Publication
(10) Pub. No.: US 2026/0250755 A1
(43) Pub. Date: Aug. 27, 2026

(54) PRIMARY TEMPLATE-DIRECTED AMPLIFICATION AND METHODS THEREOF

(71) Applicant: BioSkryb Genomics, Inc., Durham, NC (US)

(72) Inventors: Jay A.A. WEST, Durham, NC (US); Jeff BLACKINTON, Durham, NC (US)

(21) Appl. No.: 18/861,541

(22) PCT Filed: May 4, 2023
(86) PCT No.: PCT/US2023/021073

§ 371 Date: Oct. 29, 2024

Related U.S. Application Data
(60) Provisional application No. 63/338,669, filed on May 5, 2022.
(60) Provisional application No. 63/406,862, filed on Sep. 15, 2022.

(51) Int. Cl. C12Q 1/686; C12Q 1/48; C12Q 1/6827; C12Q 1/6855; C12Q 1/6869; C12Q 1/6876

(52) U.S. Cl. CPC C12Q 1/686; C12Q 1/48; C12Q 1/6827; C12Q 1/6855; C12Q 1/6869; C12Q 1/6876; C12Y 207/07; C12Q 2600/112

(57) ABSTRACT

Provided herein are compositions and methods for high-throughput Primary Template-Directed Amplification (PTA) nucleic acid amplification and sequencing methods, and their applications for mutational analysis in research, diagnostics, and treatment. Further provided herein are methods for parallel analysis of DNA, RNA, and/or proteins from single cells.

### CROSS REFERENCE

This application claims the benefit of U.S. Provisional Patent Application No. 63/338,669 filed May 5, 2022, and U.S. Provisional Patent Application No. 63/406,862 filed Sep. 15, 2022, both of which are incorporated herein by reference in their entirety.

### BACKGROUND

Research methods that utilize nucleic amplification, e.g., Next Generation Sequencing, provide large amounts of information on complex samples, genomes, and other nucleic acid sources. In some cases, these samples are obtained in small quantities from single cells. There is a need for highly accurate, scalable, and efficient nucleic acid amplification and sequencing methods for research, diagnostics, and treatment involving small samples, especially methods for high-throughput analysis.

### BRIEF SUMMARY

Provided herein are devices, methods, and systems for high-throughput sample analysis.

Provided herein are methods of amplification from single cells comprising: (a) contacting a single cell with lysis buffer, wherein the lysis buffer comprises at least one amplification primer; (b)
adding at the same time to the lysis buffer: a neutralization buffer, at least one nucleic acid polymerase, and a mixture of nucleotides, wherein the mixture of nucleotides comprises at least one terminator nucleotide which terminates nucleic acid replication by the polymerase; and (c) amplifying at least some of the genome to generate a plurality of terminated amplification products, wherein the replication proceeds by strand displacement replication, wherein if the lysis buffer or neutralization buffer comprises TWEEN-20, the amount of TWEEN-20 is no more than 0.2%. Provided herein are methods of amplification from single cells comprising: (a) isolating a single cell from a population of cells; (b) contacting the single cell with lysis buffer, wherein the lysis buffer comprises at least one amplification primer; (c) contacting the single cell with neutralization buffer, at least one nucleic acid polymerase, and a mixture of nucleotides, wherein the mixture of nucleotides comprises at least one terminator nucleotide which terminates nucleic acid replication by the polymerase; and (d) amplifying at least some of the genome to generate a plurality of terminated amplification products, wherein the replication proceeds by strand displacement replication. Further provided herein are methods wherein the lysis buffer comprises one or more of a base, a buffering agent and a chelating agent. Further provided herein are methods wherein the buffering agent comprises HEPES (4-(2-hydroxyethyl)-1-piperazineethanesulfonic acid. Further provided herein are methods wherein the concentration of one or more components of the lysis buffer ranges from about 10 mM to about 500 mM. Further provided herein are methods wherein the pH of the lysis buffer is about 11 to about 14. Further provided herein are methods wherein the step (b) is performed for more than 10 minutes. Further provided herein are methods wherein the step (b), step (c), or both step (b) and step (c) is performed for less than 10 hours. Further provided herein are methods wherein the step (b), step (c), or both step (b) and step (c) is performed for less than 1 hour. Further provided herein are methods wherein the method further comprises addition of an ERAT and/or ligation mixture directly after the step (c). Further provided herein are methods wherein the terminator is an irreversible terminator. Further provided herein are methods wherein the terminator nucleotide is selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids. Further provided herein are methods wherein the nucleotides with modification to the alpha group are alpha-thio dideoxynucleotides. Further provided herein are methods wherein the terminator nucleotide comprises modifications of the r group of the 3′ carbon of the deoxyribose. Further provided herein are methods wherein terminator nucleotide is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. Further provided herein are methods wherein the concentration of magnesium is less than 8 mM. Further provided herein are methods wherein the mixture of nucleotides comprises at least one dNTP. Further provided herein are methods wherein the at least one dNTP is present at a concentration of 0.1-5 mM. Further provided herein are methods wherein the at least one dNTP is present at a concentration of less than 2.5 mM. Further provided herein are methods wherein the at least one terminator nucleotides is present at a concentration of less than 0.3 mM. Further provided herein are methods wherein the polymerase has substantially no polymerase activity until heated to at least 40 degrees C. Further provided herein are methods wherein the method further comprises sequencing the plurality of terminated amplification products or amplicons thereof. Further provided herein are methods wherein sequencing results in one or more of: an allelic balance of at least 0.8; a 1× coverage of at least 0.95; a precision of at least 0.99; and an SNV sensitivity of at least 0.85. Further provided herein are methods wherein the single cell is NA12878. Further provided herein are methods wherein the method comprises analysis of at least 10⁰ cells. Further provided herein are methods wherein the method comprises analysis of at least 1000 cells.

Provided herein are compositions comprising at least one amplification primer, at least one nucleic acid polymerase, a buffer, and a mixture of nucleotides, wherein the mixture of nucleotides comprises at least one dNTP and at least one terminator nucleotide which terminates nucleic acid replication by the polymerase, where the buffer comprises magnesium, wherein concentration of magnesium is less than 8 mM. Further provided herein are compositions wherein the composition further comprises a target nucleic acid molecule. Further provided herein are compositions wherein magnesium concentration is 3-6 mM. Further provided herein are compositions wherein buffer comprises magnesium chloride. Further provided herein are compositions wherein the at least one dNTP is present at a concentration of 0.1-5 mM. Further provided herein are compositions wherein the at least one dNTP is present at a concentration of less than 2.5 mM. Further provided herein are compositions wherein the at least one dNTP is present at a concentration of 0.5-2 mM. Further provided herein are compositions wherein the concentration ratio of dNTPs and terminators to magnesium is 0.5-1.5. Further provided herein are compositions the at least one terminator nucleotide is present at a concentration of less than 0.3 mM. Further provided herein are compositions the at least one terminator nucleotide is present at a concentration of 0.05-0.20 mM. Further provided herein are compositions the terminator is an irreversible terminator. Further provided herein are compositions the terminator nucleotide is selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids. Further provided herein are compositions the nucleotides with modification to the alpha group are alpha-thio dideoxynucleotides. Further provided herein are compositions the terminator nucleotide comprises modifications of the r group of the 3′ carbon of the deoxyribose. Further provided herein are compositions the terminator nucleotide is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. Further provided herein are compositions the composition further comprises a lysis buffer. Further provided herein are compositions the lysis buffer comprises one or more of a base, a buffering agent and a chelating agent. Further provided herein are compositions the buffering agent comprises HEPES (4-(2-hydroxyethyl)-1-piperazineethanesulfonic acid, BES, MOPS, TES,

HEPES, DIPSO, MOBS, TAPSO, Trizma, HEPPSO, POPSO, or TEA. Further provided herein are compositions the concentration of one or more components of the lysis buffer ranges from about 10 mM to about 500 mM. Further provided herein are compositions wherein the magnesium concentration is 2-4 mM.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B illustrate a workflow for high throughput analysis of single cells using two-step PTA methods (bottom) as compared to four-step PTA methods (top).

FIG. 2A illustrates a plot of signal vs. cycles for four-step PTA comprising lysis, neutralization, priming and PTA reaction steps.

FIG. 2B illustrates a plot of signal vs. cycles for two-step PTA comprising lysis/priming and neutralization/PTA reaction steps.

FIG. 3 illustrates the yield of nucleic acids (ng) using two-step PTA. NTC means no template control. SCs means single cells. Different colors represent replicates that do not show amplification.

FIG. 4 illustrates mitochondrial reads (%) vs. pre-seq value for four-step PTA (circles with X's) vs. two-step PTA (solid circles). Optimal performance among the conditions tested are shown in the boxed region. The x-axis is labeled proportion Chromosome M from 0, 0.01, and 0.02; the y-axis represents pre-seq values from 0 to 4.5×10⁹ at 0.5×10⁹ intervals.

FIG. 5A illustrates a graph of real-time PTA showing yield (measured as fluorescence) as a function of amplification cycles for SB4B conditions using controls. The x-axis is labeled Cycle from 0 to 120 at 20 unit intervals, the y-axis is labeled Fluorescence from 0 to 500,000 at 50,000 unit intervals.

FIG. 5B illustrates a graph of real-time PTA showing yield (measured as fluorescence) as a function of amplification cycles for SB4B conditions using single cells. The x-axis is labeled Cycle from 0 to 120 at 20 unit intervals, the y-axis is labeled Fluorescence from 0 to 500,000 at 50,000 unit intervals.

FIG. 5C illustrates a graph of real-time PTA showing yield (measured as fluorescence) as a function of amplification cycles for SB4W conditions using controls. The x-axis is labeled Cycle from 0 to 120 at 20 unit intervals, the y-axis is labeled Fluorescence from 0 to 500,000 at 50,000 unit intervals.

FIG. 5D illustrates a graph of real-time PTA showing yield (measured as fluorescence) as a function of amplification cycles for SB4B conditions using single cells. The x-axis is labeled Cycle from 0 to 120 at 20 unit intervals, the y-axis is labeled Fluorescence from 0 to 500,000 at 50,000 unit intervals.

FIG. 5E illustrates a graph of real-time PTA showing yield (measured as fluorescence) as a function of amplification cycles for samples A-P. The x-axis is labeled Cycle from 0 to 120 at 20 unit intervals, the y-axis is labeled Fluorescence from 0 to 200,000 at 25,000 unit intervals.

FIG. 6A illustrates the effect of changing (top to bottom in x-axis) concentration of Mg, dNTP, ddNTP, KOH in SB4, Phi29, SEZs separate(S) or combined (C) on yield. Yield in ng/μL is shown on the y-axis from 0 to 1000 in 200 unit intervals. Different conditions are shown on the x-axis. SB4W (solid box) and SB4B (dotted box) conditions are labeled.

FIG. 6B illustrates graphs for SB4B-nom, SB4D, and SB4W conditions showing fragment sizes for SB4B-nom (top), SB4D (middle), and SB4W (bottom). The y-axis is labeled sample intensity [normalized FU] from 0 to 2000 at 1000 unit intervals; the x-axis represents sizes (bp) with 15, 100, 250, 400, 600, 1000, 1500, 2500, 3500, 5000, and 10000 labeled.

FIG. 6C illustrates the effect of changing (top to bottom in x-axis) concentration of Mg, dNTP, ddNTP, KOHin SB4, Phi29, SEZs separate(S) or combined (C) on Pre-seq value. Pre-seq values are shown on the y-axis from 0 to 4×109 in 109 unit intervals. Different conditions are shown on the x-axis. SB4W (solid box) and SB4B (dotted box) conditions are labeled.

FIG. 6D illustrates the effect of changing (top to bottom in x-axis) concentration of Mg, dNTP, ddNTP, KOHin SB4, Phi29, SEZs separate(S) or combined (C) on chimera rate. Chimera rates are shown on the y-axis from 0 to 40 in 5 unit intervals. Different conditions are shown on the x-axis. SB4W (solid box) and SB4B (dotted box) conditions are labeled.

FIG. 7A illustrates a reaction setup, including multiple NTC, 1 ng gDNA, 100 μg gDNA and 10 μg gDNA controls added into a 384-well plate containing sorted single cells. 1 μL of Cell Buffer is dispensed into the wells in columns 2 through 23. Cells are then sorted into these wells (FACS/FANS etc.) Prior to processing/PTA amplification, 1 μL of the control samples are added to columns 1 and 24 as shown.

FIG. 8A depicts a graph of raw DNA yield (ng) after amplification using a 2-step PTA reaction. The y-axis is labeled yield from 0 to 400 ng at 100 unit intervals.

FIG. 8B depicts a graph of fragment sizes obtained from a 2-step PTA reaction. The y-axis is labeled sample intensity 0 to 2000 normalized FU at 500 unit intervals. The x-axis is labeled size (bp) at 15, 100, 250, 400, 600, 1000, 1500, 2500, 3500, 5000, and 10000.

FIG. 9 illustrates a graph of PreSeq data from single cells using a variety of reaction buffer formulations (L1-L5G). The y-axis is labeled PreSeq (estimated Genome size) from 1×10⁹ to 4×10⁹ at 1×10 intervals. The x-axis is labeled reaction conditions as L1, L2, L3, L4, L5F, and L5G. The key represents varying magnesium ion concentrations in reaction buffer SB5: circles (2.2 mM), up-pointing triangles (2.2+0.2 mM), down-pointing triangles (2.2+0.3 mM), and diamonds (2.45 mM).

FIG. 10A depicts a plot of amplification time for a two-step vs. four step PTA method. Time for the 2-step method is reduced by ~4×, to 2.5 hrs.

FIG. 10B depicts a plot of yields for a two-step vs. four step PTA method. Both methods yielded sufficient amplified genome to prepare NGS libraries.

FIGS. 11A-11D depict plots of sequencing metrics for the four-step (V1) and two-step (V2) method. The allelic balance of the reaction was both higher and had less deviation in either operator mode (automated vs manual, FIG. 11A). Genome coverage at 1× was also improved (FIG. 11B), which has the downstream effect of improving the overall sensitivity of single nucleotide variant detection (FIG. 11C), which the precision of the detected event was unaffected (FIG. 11D).

### DETAILED DESCRIPTION OF THE INVENTION

There is a need to develop new scalable, accurate and efficient methods for nucleic acid amplification (including single-cell and multi-cell genome amplification) and sequencing which would overcome limitations in the current methods by increasing sequence representation, uniformity and accuracy in a reproducible manner. Provided herein are compositions and methods for providing accurate and scalable Primary Template-Directed Amplification (PTA) and sequencing. Further provided herein are methods of high-throughput PTA. Further provided herein are methods of multiomic analysis, including analysis of proteins, DNA, and RNA from single cells, and corresponding post-transcriptional or post-translational modifications in combination with PTA. Especially for high throughput applications, a need exists for streamlined workflows which reduce processing times.

### Definitions

Unless defined otherwise, all technical and scientific terms used herein have the same meaning as is commonly understood by one of ordinary skill in the art to which these inventions belong.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

The terms “subject” or “patient” or “individual”, as used herein, refer to animals, including mammals, such as, e.g., humans, veterinary animals (e.g., cats, dogs, cows, horses, sheep, pigs, etc.) and experimental animal models of diseases (e.g., mice, rats). In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A practical Approach, Volumes I and II (D. N. Glover ed. 1985); Oligonucleotide Synthesis (MJ. Gait ed. 1984); Nucleic Acid Hybridization (B. D. Hames & S. J. Higgins eds. (1985»>; Transcription and Translation (B. D. Hames & S. J. Higgins, eds. (1984»; Animal Cell Culture (R. I. Freshney, ed. (1986»; Immobilized Cells and Enzymes (IRL Press, (1986»; B. Perbal, A practical Guide To Molecular Cloning (1984); F. M. Ausubel et al. (eds.), Current Protocols in Molecular Biology, John Wiley & Sons, Inc. (1994); among others.

The term “nucleic acid” encompasses multi-stranded, as well as single-stranded molecules. In double- or triple-stranded nucleic acids, the nucleic acid strands need not be coextensive (i.e., a double-stranded nucleic acid need not be double-stranded along the entire length of both strands). Nucleic acid templates described herein may be any size depending on the sample (from small cell-free DNA fragments to entire genomes), including but not limited to 50-300 bases, 100-2000 bases, 100-750 bases, 170-500 bases, 100-5000 bases, 50-10,000 bases, or 50-2000 bases in length. In some instances, templates are at least 50, 100, 200, 500, 1000, 2000, 5000, 10,000, 20,000 50,000, 100,000, 200,000, 500,000, 1,000,000 or more than 1,000,000 bases in length. Methods described herein provide for the amplification of nucleic acid acids, such as nucleic acid templates. Methods described herein additionally provide for the generation of isolated and at least partially purified nucleic acids and libraries of nucleic acids. In some instances, methods described herein provide for extracted nucleic acids (e.g., extracted from tissues, cells, or media). Nucleic acids include but are not limited to those comprising DNA, RNA, circular RNA, mtDNA (mitochondrial DNA), cfDNA (cell free DNA), cfRNA (cell free RNA), siRNA (small interfering RNA), cffDNA (cell free fetal DNA), mRNA, tRNA, IRNA, miRNA (microRNA), synthetic polynucleotides, polynucleotide analogues, any other nucleic acid consistent with the specification, or any combinations thereof. The length of polynucleotides, when provided, are described as the number of bases and abbreviated, such as nt (nucleotides), bp (bases), kb (kilobases), or Gb (gigabases).

The term “droplet” as used herein refers to a volume of liquid on a droplet actuator. Droplets in some instances, for example, be aqueous or non-aqueous or may be mixtures or emulsions including aqueous and non-aqueous components. For non-limiting examples of droplet fluids that may be subjected to droplet operations, see, e.g., Int. Pat. Appl. Pub. No. WO2007/120241. Any suitable system for forming and manipulating droplets can be used in the embodiments presented herein. For example, in some instances a droplet actuator is used. For non-limiting examples of droplet actuators which can be used, see, e.g., U.S. Pat. Nos. 6,911,132, 6,977,033, 6,773,566, 6,565,727, 7,163,612, 7,052,244, 7,328,979, 7,547,380, 7,641,779, U.S. Pat. Appl. Pub. Nos. US20060194331, US20030205632, US20060164490, US20070023292, US20060039823, US20080124252, US20090283407, US20090192044, US20050179746, US20090321262, US20100096266, US20110048951, Int. Pat. Appl. Pub. No. WO2007/120241. In some instances, beads are provided in a droplet, in a droplet operations gap, or on a droplet operations surface. In some instances, beads are provided in a reservoir that is external to a droplet operations gap or situated apart from a droplet operations surface, and the reservoir may be associated with a flow path that permits a droplet including the beads to be brought into a droplet operations gap or into contact with a droplet operations surface. Non-limiting examples of droplet actuator techniques for immobilizing magnetically responsive beads and/or non-magnetically responsive beads and/or conducting droplet operations protocols using beads are described in U.S. Pat. Appl. Pub. No. US20080053205, Int. Pat. Appl. Pub. No. WO2008/098236, WO2008/134153, WO2008/116221, WO2007/120241. Bead characteristics may be employed in the multiplexing embodiments of the methods described herein. Examples of beads having characteristics suitable for multiplexing, as well as methods of detecting and analyzing signals emitted from such beads, may be found in U.S. Pat. Appl. Pub. No. US20080305481, US20080151240, US20070207513, US20070064990, US20060159962, US20050277197, US20050118574.

Primers and/or template switching oligonucleotides can also be affixed to solid substrate to facilitate reverse transcription and template switching of the mRNA polynucleotides. In this arrangement a portion of the RT or template switching reaction occurs in the bulk solution of the device, where the second step of the reaction occurs in proximity to the surface. In other arrangements the primer of template switch oligonucleotide is allowed to be released from the solid substrate to allow the entire reaction to occur above the surface in the solution. In a polyomic approach the primers for the multistage reaction in some instances is affixed to the solid substrate or combined with beads to accomplish combinations of multistage primers.

Certain microfluidic devices also support polyomic approaches. Devices fabricated in PDMS, as an example, often have contiguous chambers for each reaction step. Such multichambered devices are often segregated using a microvalve structure which can be controlled though the pressure with air, or a fluid such as water or inert hydrocarbon (i.e. fluorinert). In a multiomic approach each stage of the reaction can be sequestered and allowed to be conducted discretely. At the completion of a particular stage a valve between an adjacent chamber can be released on the substrates for the subsequent reaction can be added in a serial fashion. The result is the ability to emulate an sequential set of reactions, such as a multiomic (Protein/RNA/DNA/epigenomic) set of reactions using an individual cell as a input template material. Various microfluidics platforms may be used for analysis of single cells. Cells in some instances are manipulated through hydrodynamics (droplet microfluidics, inertial microfluidics, vortexing, microvalves, microstructures (e.g., microwells, microtraps)), electrical methods (dielectrophoresis (DEP), electroosmosis), optical methods (optical tweezers, optically induced dielectrophoresis (ODEP), opto-thermocapillary), acoustic methods, or magnetic methods. In some instances, the microfluidics platform comprises microwells. In some instances, the microfluidics platform comprises a PDMS (Polydimethylsiloxane)-based device. Non-limited examples of single cell analysis platforms compatible with the methods described herein are: ddSEQ Single-Cell Isolator, (Bio-Rad, Hercules, CA, USA, and Illumina, San Diego, CA, USA)); Chromium (10× Genomics, Pleasanton, CA, USA)); Rhapsody Single-Cell Analysis System (BD, Franklin Lakes, NJ, USA); Tapestri Platform (MissionBio, San Francisco, CA, USA)), Nadia Innovate (Dolomite Bio, Royston, UK); C1 and Polaris (Fluidigm, South San Francisco, CA, USA); ICELL8 Single-Cell System (Takara); MSND (Wafergen); Puncher platform (Vycap); CellRaft AIR System (CellMicrosystems); DEPArray NXT and DEP Array System (Menarini Silicon Biosystems); AVISO CellCelector (ALS); and InDrop System (1CellBio), and TrapTx (Celldom).

As used herein, the term “unique molecular identifier (UMI)” refers to a unique nucleic acid sequence that is attached to each of a plurality of nucleic acid molecules. When incorporated into a nucleic acid molecule, an UMI in some instances is used to correct for subsequent amplification bias by directly counting UMIs that are sequenced after amplification. The design, incorporation and application of UMIs is described, for example, in Int. Pat. Appl. Pub. No. WO 2012/142213, Islam et al. Nat. Methods (2014) 11:163-166, Kivioja, T. et al. Nat. Methods (2012) 9:72-74, Brenner et al. (2000) PNAS 97 (4), 1665, and Hollas and Schuler, (2003) Conference: 3rd International Workshop on Algorithms in Bioinformatics, Volume: 2812.

As used herein, the term “barcode” refers to a nucleic acid tag that can be used to identify a sample or source of the nucleic acid material. Thus, where nucleic acid samples are derived from multiple sources, the nucleic acids in each nucleic acid sample are in some instances tagged with different nucleic acid tags such that the source of the sample can be identified. Barcodes, also commonly referred to indexes, tags, and the like, are well known to those of skill in the art. Any suitable barcode or set of barcodes can be used. See, e.g., non-limiting examples provided in U.S. Pat. No. 8,053,192 and Int. Pat. Appl. Pub. No. WO2005/068656. Barcoding of single cells can be performed as described, for example, in U.S. Pat. Appl. Pub. No. 2013/0274117.

The terms “solid surface,” “solid support” and other grammatical equivalents herein refer to any material that is appropriate for or can be modified to be appropriate for the attachment of the primers, barcodes and sequences described herein. Exemplary substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon, nitrocellulose, ceramics, resins, silica, silica-based materials (e.g., silicon or modified silicon), carbon, metals, inorganic glasses, plastics, optical fiber bundles, and a variety of other polymers. In some embodiments, the solid support comprises a patterned surface suitable for immobilization of primers, barcodes and sequences in an ordered pattern.

As used herein, the term “biological sample” includes, but is not limited to, tissues, cells, biological fluids and isolates thereof. Cells or other samples used in the methods described herein are in some instances isolated from human patients, animals, plants, soil or other samples comprising microbes such as bacteria, fungi, protozoa, etc. In some instances, the biological sample is of human origin. In some instances, the biological is of non-human origin. The cells in some instances undergo PTA methods described herein and sequencing. Variants detected throughout the genome or at specific locations can be compared with all other cells isolated from that subject to trace the history of a cell lineage for research or diagnostic purposes. In some instances, variants are confirmed through additional methods of analysis such as direct PCR sequencing.

### High-Throughput Primary Template-Directed Amplification

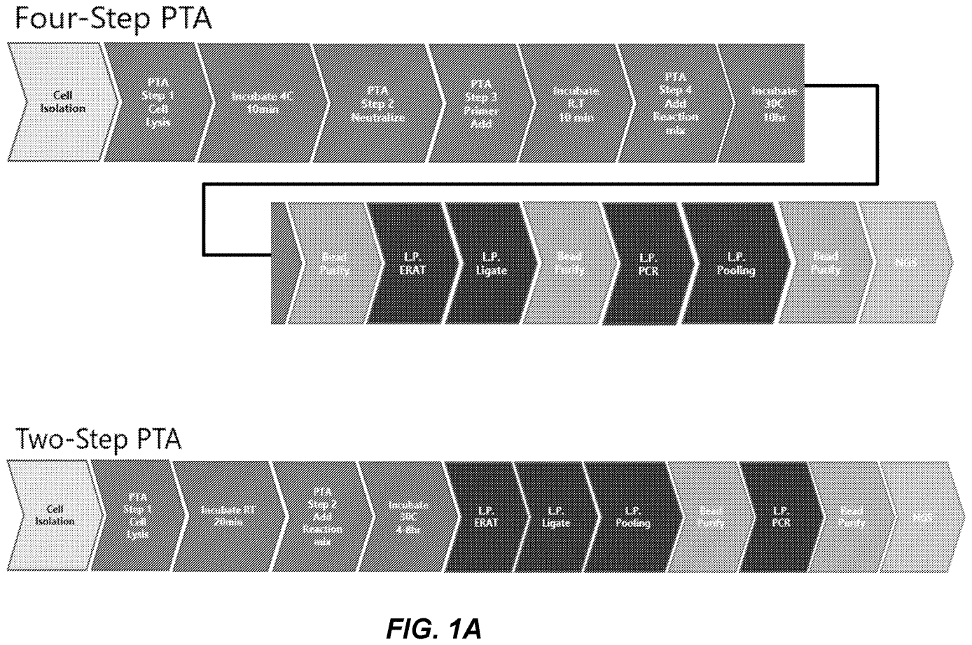

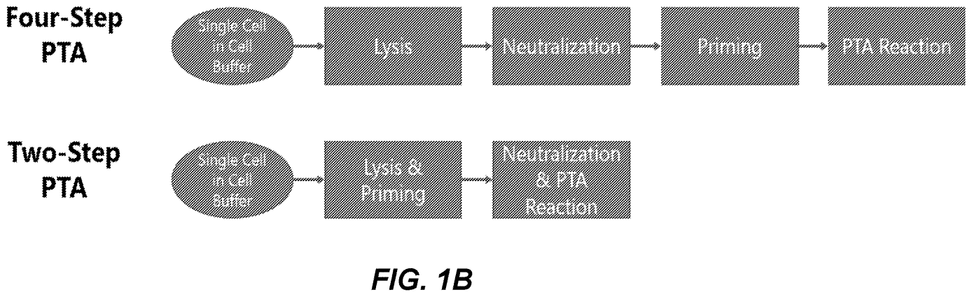

Further provided herein are methods for high-throughput primary template-directed amplification (PTA). Templates in some instances comprise DNA, RNA, or single cells. In some instances, the methods comprise one, two, three, four, or five steps. In some instances, the methods comprise two-step PTA methods. As shown in FIGS. 1A and 1B, two-step PTA methods comprises lysis (step 1) and reaction mix addition (step 2). Compared to four-step PTA methods, in some instances two-step PTA methods combine lysis and priming steps into a single step (step 1) and neutralization and reaction mix addition steps into a single step (step 2), respectively. In some instances, this eliminates need for post-PTA clean-up to add ERAT/ligation directly to amplified products. Also, two-step PTA methods can pool many reactions into single tube for purification and PCR. In some instances, all reagents used in a step are added together to a reaction mixture. In some instances, all reagents used in a step are added together to the reaction mixture from the previous step (i.e., sequential addition). Processing large numbers of samples processed at the same time may require many liquid handling steps as well as extended setup times, which can contribute to reduced efficiencies and time delay between samples. Sequential addition in some instances both decreases the number of liquid handling steps, as well as reduces overall workflow times.

Provided herein are methods comprising one or more steps of contacting a single cell with lysis buffer, wherein the lysis buffer comprises at least one amplification primer; adding at the same time to the lysis buffer: a neutralization buffer, at least one nucleic acid polymerase, and a mixture of nucleotides, wherein the mixture of nucleotides comprises at least one terminator nucleotide which terminates nucleic acid replication by the polymerase; and amplifying at least some of the genome to generate a plurality of terminated amplification products, wherein the replication proceeds by strand displacement replication. In some instances, when the lysis buffer or neutralization buffer comprises TWEEN-20, the amount of TWEEN-20 is no more than 0.2% (v/v). In some instances, the amount of Tween-20 in a reaction mixture is no more than 0.5, 0.4, 0.3, 0.2, 0.15, 0.1, 0.5, or no more than 0.1% (v/v).

In some instances, a lysis buffer is used with the methods described herein. In some instances, the buffer pH is about 11, 11.5, 12, 12.5, 13, 13.5, or about 14. In some instances, the buffer pH is 12-14, 12.5-14, 12.5-13.5, or 13-14. In some instances, the buffer pH is at least 11.5, 12, 12.5, 13, or at least 13.5. In some instances, the concentration of the lysis buffer is at least about 10, 20, 30, 40, 50, 60, 70. 80, 90, 100, or more than 100 mM. In some instances, the concentration of the lysis buffer is at least about 100, 200, 300, 400, 500, or more than 500 mM. In some embodiments, the concentration of the lysis buffer is about 10 mM and about 100 mM, about 20 mM and about 400 mM, 30 mM and about 300 mM, about 40 mM and about 200 mM, about 50 mM and about 100 mM. In some instances, the concentration of the lysis buffer in two-step PTA methods is higher than four-step PTA methods. In some instances, the concentration of the lysis buffer in two-step PTA methods is comparable to four-step PTA methods.

In some instances, the buffers comprise surfactants/detergent or denaturing agents (Tween-20, Triton X-1-- (e.g., 100, 114, etc.), Igepal or NP-40, DMSO, DMF, pegylated polymers comprising a hydrophobic group, or other surfactant), salts (potassium or sodium phosphate (monobasic or dibasic), sodium chloride, potassium chloride, TrisHCI, magnesium chloride or sulfate, Ammonium salts such as phosphate, nitrate, or sulfate, EDTA), reducing agents (DTT, THP, DTE, beta-mercaptoethanol, TCEP, or other reducing agent) or other components (glycerol, hydrophilic polymers such as PEG). In some instances, the lysis buffer is a sulfonic acid buffering agent. In some instances, the lysis buffer is a zwitterionic sulfonic acid buffering agent. In some instances, the lysis buffer is BES, MOPS, TES, HEPES (4-(2-hydroxyethyl)-1-piperazineethanesulfonic acid), DIPSO, MOBS, TAPSO, Trizma, HEPPSO, POPSO, or TEA. In some instances, the lysis buffer is HEPES (4-(2-hydroxyethyl)-1-piperazineethanesulfonic acid). In some instances, the lysis buffer comprises a base, or other reagent described herein for cell lysis. In some instances, a reagent or buffer described herein comprises a surfactant/denaturing agent. In some instances, a surfactant/denaturing agent comprises Tween-20, Triton X-1-- (e.g., 100, 114, etc.), Igepal or NP-40, DMSO, DMF, pegylated polymers comprising a hydrophobic group, or other surfactant. In some instances, the amount of the surfactant/denaturing agent is 0.1-5%, 0.2-5%, 0.3-5%, 0.3-4%, 0.3-3%, 0.2-2%, 0.1-0.5%, 0.1-3%, 0.1-2%, 0.2-5%, or 0.2-0.8% (v/v). In some instances, the amount of Tween-20 in a buffer described herein is no more than 0.5, 0.4, 0.3, 0.2, 0.15, 0.1, 0.5, or no more than 0.1% (v/v).

In some instances, the temperature of the lysis buffer is 1-30 degrees C. In some instances, the temperature of the lysis buffer is no more than 10, 15, 20, 25, or no more than 30 degrees C. In some instances, the temperature of the lysis buffer is about 2, 5, 10, 15, 20, 25, or about 30 degrees C.

In some instances, the lysis buffer further comprise at least one primer. In some instances, a primer comprises a phosphonothioate linkage. In some instances, the residence time for a sample in the lysis buffer is more than about 5, 10, 15, 20, 25, 30 or more than 30 minutes. In some instances, the residence time for a sample in the lysis buffer is no more than 60, 50, 40, 30, 20, 10 minutes, or no more than 10 minutes.

In some instances, a reaction mixture is used for a PTA reaction. In some instances, the temperature of the reaction mixture is 12-30, 10-30, 15-30, 15-25, 17-23, or 20-30 degrees C. In some instances, the temperature of the reaction mixture is about 15, 17, 20, 22, 25, 27, or about 30 degrees C. In some instances, the temperature of the reaction mixture starts at a first temperature at the beginning of PTA and is later cooled to a second temperature after the PTA reaction completes. In some instances, the temperature of the reaction mixture starts at 30 degrees C. at the beginning of PTA and is cooled to 4-10 degrees C. after the PTA reaction completes. In some instances, the PTA reaction is measured in real-time using a dye. In some instances, the dye is an intercalating dye. In some instances, the residence time for a sample in the reaction mixture is no more than 10, 8, 6, 4, 3, or no more than 2 hours. In some instances, the residence time for a sample in the reaction chamber mixture is about 10, 8, 6, 4, 3, or about 2 hours. In some instances, the residence time for a sample in the reaction mixture is 2-10 hours, 2-8 hours, 1-8 hours, 4-10 hours, 4-8 hours, or 6-10 hours. In some instances, the residence time for a sample in the reaction mixture is determined based on the amount of amplification product produced. In some instances, the residence time for a sample in the reaction mixture is such that about 50, 60, 75, 80, 90, 100, 110, 125, 150, 200, or 500 ng of amplification product is produced. In some instances, the residence time for a sample in the reaction mixture is such that about 50-500, 50-200, 75-125, 50-100, 75-150, 100-250, 100-500, or 200-500 ng of amplification product is produced. In some instances, a reaction mixture comprises a neutralization buffer. In some instances, the neutralization buffer comprises an acid, or other reagent described herein for neutralization. In some instances, the buffer pH is about 0.5, 1, 1.5, 2, 2.5, 3, 3.5, or about 4. In some instances, the buffer pH is 0.5-5, 0.5-4, 0.5-3, 0.5-2.5, 0.5-2, or 1-3. In some instances, a reaction mixture comprises a surfactant/denaturing agent. In some instances, a surfactant/denaturing agent comprises Tween-20, Triton X-1-- (e.g., 100, 114, etc.), Igepal or NP-40, DMSO, DMF, pegylated polymers comprising a hydrophobic group, or other surfactant. In some instances, the amount of the surfactant/denaturing agent is 0.1-5%, 0.2-5%, 0.3-5%, 0.3-4%, 0.3-3%, 0.2-2%, 0.1-0.5%, 0.1-3%, 0.1-2%, 0.2-5%, or 0.2-0.8% (v/v).

In some instances, the buffers comprise one or more metal ions. In some instances, metal ions are present as salts. In some instances, metal ions comprise magnesium, potassium, calcium, sodium, manganese, or lithium. In some instances, salts comprise a counterion. In some instances, salts comprise an inorganic counterion. In some instances, the counterion comprises chloride, bromide, iodide, hydrogen sulfate, sulfate, phosphate, hydrogen phosphate, carbonate, bicarbonate, or hexafluorophosphate. In some instances, salts comprise an organic counterion. In some instances, the counterion comprises acetate, propionate, or citrate. In some instances, the metal ion is present in the PTA reaction at a concentration of 0.5-20, 0.5-15, 0.5-10, 0.5-9, 0.5-6, 1-20, 1-15, 1-10, 2-15, 2-10, 5-20, 5-15, 2-5, 1-5, 0.5-5, 0.05-6, 2-6, or 3-6 mM. In some instances, the metal ion is present in the PTA reaction at a concentration of no more than 10, 9, 8, 7, 6, 5, 4, 3, 2, 1, 0.5, or no more than 0.2 mM. In some instances, the metal ion comprises magnesium. In some instances, magnesium is present in the PTA reaction at a concentration of 0.5-20, 0.5-15, 0.5-10, 0.5-9, 0.5-6, 1-20, 1-15, 1-10, 2-15, 2-10, 5-20, 5-15, 2-5, 1-5, 0.5-5, 0.05-6, 2-6, or 3-6 mM. In some instances, magnesium is present in the PTA reaction at a concentration of no more than 10, 9, 8, 7, 6, 5, 4, 3, 2, 1, 0.5, or no more than 0.2 mM. In some instances, magnesium is used at a ratio relative to NTPs (e.g., dNTPs, terminators). In some instances the concentration ratio of dNTPs and terminators to magnesium is 0.5-1.5, 0.1-2, 0.3-2, 0.4-1.7, 0.5-1.3, 0.7-1.2, 0.8-1.3, 0.8-1.5, 0.5-3, 0.5-2.5, 0.5-1.2, or 1-3. In some instances the concentration ratio of dNTPs to magnesium is 0.5-1.5, 0.1-2, 0.3-2, 0.4-1.7, 0.5-1.3, 0.7-1.2, 0.8-1.3, 0.8-1.5, 0.5-3, 0.5-2.5, 0.5-1.2, or 1-3. In some instances the concentration ratio of terminators to magnesium is 0.5-1.5, 0.1-2, 0.3-2, 0.4-1.7, 0.5-1.3, 0.7-1.2, 0.8-1.3, 0.8-1.5, 0.5-3, 0.5-2.5, 0.5-1.2, or 1-3.

A PTA reaction assembled from multiple steps (e.g., two step process) may comprise a number of different reagents.

The PTA reaction may comprise a buffer. In some instances, a buffer comprises a cell buffer, lysis buffer, or other buffer. In some instances, a cell buffer remains from cell sorting or other cell acquisition process. In some instances, a cell buffer is present at 0.1-0.5, 0.05-0.3, 0.05-0.5, 0.2-0.3, 0.2-0.5, or 0.2-0.8 mM. In some instances, PTA reaction comprises BES, MOPS, TES, HEPES (4-(2-hydroxyethyl)-1-piperazineethanesulfonic acid), DIPSO, MOBS, TAPSO, Trizma, HEPPSO, POPSO, or TEA. In some instances, BES, MOPS, TES, HEPES (4-(2-hydroxyethyl)-1-piperazineethanesulfonic acid), DIPSO, MOBS, TAPSO, Trizma, HEPPSO, POPSO, or TEA is present at a concentration of 50-300, 50-200, 50-150, 100-300, 100-200, 100-150, 125-150, 115-145, or 125-300 mM.

The PTA reaction may comprise a salt. In some instances, the salt comprises sodium. In some instances, the salt comprises a chloride anion. In some instances the concentration of sodium or chloride anion is 20-50, 10-60 5-60, 10-30, 20-40, 30-40, or 30-60 mM. In some instances the salt comprises ammonium sulfate. In some instances, the concentration of ammonium sulfate is 10-50, 10-60 5-60, 10-30, 10-40, 5-15, or 15-50 mM. In some instances, the salt comprises magnesium. In some instances, the concentration of magnesium is 1-10, 1-8, 1-6, 2-8, 2-6, or 4-5 mM. In some instances, the salt comprises EDTA. In some instances, the concentration of EDTA is 0.1-0.5, 0.05-0.3, 0.05-0.5, 0.2-0.3, 0.2-0.5, or 0.2-0.8 mM.

The PTA reaction may comprise a base. In some instances, the base comprises hydroxide. In some instances, the base is present at a concentration of 10-100, 10-80, 10-70, 10-50, 25-100, 25-75, 30-60, 40-75, or 40-60 mM. In some instances, the base comprises a hydroxide and is present at a concentration of 10-100, 10-80, 10-70, 10-50, 25-100, 25-75, 30-60, 40-75, or 40-60 mM.

The PTA reaction may comprise a reducing agent. In some instances, the reducing agent comprises DTT. In some instances, the reducing agent is present at a concentration of 5-20, 5-15, 5-10, 7-20, 7-15, 7-10, 9-12, or 10-20 mM.

The PTA reaction may comprise a reporter dye. In some instances the dye comprises Evagreen, SYBR green, or other dye. In some instances, the dye is used at 0.01-0.1, 0.01-0.2, 0.02-2, 0.05-0.1, 0.05-0.2 or 0.8-0.9 equivalents.

The PTA reaction may comprise a surfactant/denaturing agent. In some instances, the surfactant/denaturing agent comprises Tween-20, Triton X-1-- (e.g., 100, 114, etc.), Igepal or NP-40. In some instances, the concentration of surfactant is 0.01-1.0, 0.01-0.8, 0.01-0.7, 0.5-0.5, 0.1-0.4, 0.1-0.3, or 0.05-0.3 (v/v). In some instances, the amount of the surfactant/denaturing agent is 0.1-5%, 0.2-5%, 0.3-5%, 0.3-4%, 0.3-3%, 0.2-2%, 0.1-0.5%, 0.1-3%, 0.1-2%, 0.2-5%, or 0.2-0.8% (v/v). In some instances, the concentration of Tween-20 is 0.01-1.0, 0.01-0.8, 0.01-0.7, 0.5-0.5, 0.1-0.4, 0.1-0.3, or 0.05-0.3% (v/v). In some instances, the amount of the concentration of Tween-20 is 0.1-5%, 0.2-5%, 0.3-5%, 0.3-4%, 0.3-3%, 0.2-2%, 0.1-0.5%, 0.1-3%, 0.1-2%, 0.2-5%, or 0.2-0.8% (v/v). In some instances, the amount of Tween-20 in a buffer described herein is no more than 0.5, 0.4, 0.3, 0.2, 0.15, 0.1, 0.5, or no more than 0.1% (v/v).

The PTA reaction may comprises dNTPs. In some instances dNTPs comprise dATP, dTTP, dGTP, or dCTP. In some instances, the concentration of a dNTP is 0.4-0.8, 0.2-0.8. 0.3-0.8, 0.4-0.8, 0.5-0.7, 0.6-1.0, 0.6-0.8 or 0.7-1.5 mM. In some instances, the PTA reaction comprises terminators. In some instances terminators comprise ddATP, ddTTP, ddGTP, or ddCTP. In some instances terminators comprise alpha-thio ddATP, ddTTP, ddGTP, or ddCTP. In some instances the concentration of a terminator is 0.01-0.2, 0.01-0.15, 0.05-0.15, 0.05-0.1, 0.05-0.2, or 0.75-1.25 mM.

One or more steps may be used with the methods described herein. In some instances, samples or reagents move through reaction chambers. In some instances, samples are treated sequentially by addition of different reagents at each step. In some instances, samples are treated sequentially by addition of different reagents at each step without purification between steps.

In a first step, beads may be transferred to bead capture sites. In some instances, each bead capture site captures one bead. A bead is moved from the capture site through a capillary (e.g., channel) into an analyte addition chamber. In some instances, cells or other sample are released from the beads. In some instances, EDTA is used to release cells from calcium alginate beads to form droplets. In some instances, the analyte addition chamber comprises about 0.5, 1, 1.5, 2, 2.5, 3, 3.5, 4, 4.5, 5, 6, or about 7 nanoliters of liquid. In some instances, the analyte addition chamber comprises no more than 0.5, 1, 1.5, 2, 2.5, 3, 3.5, 4, 4.5, 5, 6, or no more than 7 nanoliters of buffer. In some instances, the analyte addition chamber comprises 0.5-10, 0.5-7, 0.5-5, 0.5-3, 1-4, or 2-4 nanoliters of buffer. In some instances the buffer pH is about 5.5, 6, 6.5, 7, 7.5, 8, or about 8.5. In some instances the buffer pH is 5.5-8.5, 5.5-8, 6-8, 6.5-7.5, or 7-8. In some instances, the temperature of the analyte addition chamber is 1-10 degrees C. In some instances, the temperature of the analyte addition chamber is no more than 10 degrees C. In some instances, the temperature of the lysis chamber is about 2, 4, 5, 7, 9, or about 10 degrees C. In some instances, the residence time for a sample in the analyte addition chamber is no more than 10 min, 7 min, 5 min, 4 min, 3 min, or no more than 3 min.

In a second step, with the external valve closed, the droplet is moved into a lysis chamber. In some instances, the lysis chamber comprises at least one primer. In some instances, a primer comprises a phosphonothioate linkage. In some instances, primers are pre-spotted in the chamber and optionally dried prior to addition of droplets to the device. In some instances, the lysis chamber comprises about 0.5, 1, 1.5, 2, 2.5, 3, 3.5, 4, 4.5, 5, 6, or about 7 nanoliters of liquid. In some instances, the lysis chamber comprises no more than 0.5, 1, 1.5, 2, 2.5, 3, 3.5, 4, 4.5, 5, 6, or no more than 7 nanoliters of buffer. In some instances, the lysis chamber comprises 0.5-10, 0.5-7, 0.5-5, 0.5-3, 1-4, or 2-4 nanoliters of buffer. In some instances the buffer pH is about 11, 11.5, 12, 12.5, 13, 13.5, or about 14. In some instances the buffer pH is 12-14, 12.5-14, 12.5-13.5, or 13-14. In some instances the buffer pH is at least 11.5, 12, 12.5, 13, or at least 13.5. In some instances, the lysis buffer comprises a base, or other reagent described herein for cell lysis.

In some instances, the lysis chamber comprises a lysis buffer. In some instances, the lysis buffer comprises surfactants/detergent or denaturing agents (Tween-20, Triton X-1-- (e.g., 100, 114, etc.), Igepal or NP-40, DMSO, DMF, pegylated polymers comprising a hydrophobic group, or other surfactant), salts (potassium or sodium phosphate (monobasic or dibasic), sodium chloride, potassium chloride, TrisHCI, magnesium chloride or sulfate, Ammonium salts such as phosphate, nitrate, or sulfate, EDTA), reducing agents (DTT, THP, DTE, beta-mercaptoethanol, TCEP, or other reducing agent) or other components (glycerol, hydrophilic polymers such as PEG). In some instances, the lysis buffer is a sulfonic acid buffering agent. In some instances, the lysis buffer is a zwitterionic sulfonic acid buffering agent. In some instances, the lysis buffer is HEPES (4-(2-hydroxyethyl)-1-piperazineethanesulfonic acid). In some instances, the lysis buffer comprises a base, or other reagent described herein for cell lysis. In some instances, the concentration of the lysis buffer is at least about 100, 200, 300, 400, 500, or more than 500 mM. In some embodiments, the concentration of the lysis buffer is about 10 mM and about 100 mM, about 20 mM and about 400 mM, 30 mM and about 300 mM, about 40 mM and about 200 mM, about 50 mM and about 100 mM. In some instances, the temperature of the lysis chamber is 1-30 degrees C. In some instances, the temperature of the lysis chamber is no more than 10, 15, 20, 25, or no more than 30 degrees C. In some instances, the temperature of the lysis chamber is about 2, 5, 10, 15, 20, 25, or about 30 degrees C. In some instances, the residence time for a sample in the lysis chamber is no more than 10 min, 7 min, 5 min, 4 min, 3 min, or no more than 3 min. In some instances, the residence time for a sample in the lysis chamber is more than about 5, 10, 15, 20, 25, 30, or more than 30 minutes. In some instances, the residence time for a sample in the lysis chamber is no more than about 60, 50, 40, 30, 20, 10 minutes, or no more than 10 minutes. In some instances, a lysis chamber comprises one or more primers.

In a third step, with the external valve closed, droplet is moved into a neutralization chamber. In some instances, the neutralization chamber comprises at least one primer used for a PTA reaction, such as reversible or irreversible primers. In some instances, an irreversible primer comprises a phosphonothioate linkage. In some instances, primers are pre-spotted in the chamber and optionally dried prior to addition of droplets to the device. In some instances, the neutralization chamber comprises about 0.5, 1, 1.5, 2, 2.5, 3, 3.5, 4, 4.5, 5, 6, or about 7 nanoliters of liquid. In some instances, the neutralization chamber comprises no more than 0.5, 1, 1.5, 2, 2.5, 3, 3.5, 4, 4.5, 5, 6, or no more than 7 nanoliters of buffer. In some instances, the neutralization chamber comprises 0.5-10, 0.5-7, 0.5-5, 0.5-3, 1-4, or 2-4 nanoliters of buffer. In some instances the buffer pH is about 0.5, 1, 1.5, 2, 2.5, 3, 3.5, or about 4. In some instances the buffer pH is 0.5-5, 0.5-4, 0.5-3, 0.5-2.5, 0.5-2, or 1-3. In some instances the buffer pH is no more than 5, 4, 3, 2.5, 2, 1.5, 1, or no more than 0.5. In some instances, the neutralization buffer comprises an acid, or other reagent described herein for neutralization of a lysis buffer. In some instances, the temperature of the neutralization chamber is 10-30, 15-30, 15-25, 17-23, or 20-30 degrees C. In some instances, the temperature of the neutralization chamber is about 15, 17, 20, 22, 25, 27, or about 30 degrees C. In some instances, the residence time for a sample in the neutralization chamber is no more than 10 min, 7 min, 5 min, 4 min, 3 min, or no more than 3 min. In some instances, the residence time for a sample in the neutralization chamber is no more than 10, 8, 6, 4, 3, or no more than 2 hours. In some instances, the residence time for a sample in the neutralization chamber is about 10, 8, 6, 4, 3, or about 2 hours. In some instances, the residence time for a sample in the neutralization chamber is 2-10 hours, 2-8 hours, 1-8 hours, 4-10 hours, 4-8 hours, or 6-10 hours. In some instances, reagents for neutralization and PTA amplification are used in a single chamber.

In a fourth step, with the external valve closed, the droplet is moved into a primer addition chamber. In some instances, the primer addition chamber comprises about 0.5, 1, 1.5, 2, 2.5, 3, 3.5, 4, 4.5, 5, 6, or about 7 nanoliters of liquid. In some instances, the primer addition chamber comprises no more than 0.5, 1, 1.5, 2, 2.5, 3, 3.5, 4, 4.5, 5, 6, or no more than 7 nanoliters of buffer. In some instances, the primer addition chamber comprises 0.5-10, 0.5-7, 0.5-5, 0.5-3, 1-4, or 2-4 nanoliters of buffer. In some instances the buffer pH is about 5.5, 6, 6.5, 7, 7.5, 8, or about 8.5. In some instances the buffer pH is 5.5-8.5, 5.5-8, 6-8, 6.5-7.5, or 7-8. In some instances, the primer addition chamber comprises primers used for a PTA reaction, such as reversible or irreversible primers. In some instances, an irreversible primer comprises a phosphonothioate linkage. In some instances, the temperature of the primer addition chamber is 10-30, 15-30, 15-25, 17-23, or 20-30 degrees C. In some instances, the temperature of the primer addition chamber is about 15, 17, 20, 22, 25, 27, or about 30 degrees C. In some instances, primers are pre-spotted in the chamber and optionally dried prior to addition of droplets to the device. In some instances, the residence time for a sample in the primer addition chamber is no more than 10 min, 7 min, 5 min, 4 min, 3 min, or no more than 3 min.

In a fifth step, with the external valve closed, the droplet is moved into a reaction (mix) chamber. In some instances the PTA reaction is conducted inside the reaction chamber. In some instances, the reaction chamber comprises about 5, 5.5, 6, 6.5, 7, 7.5, 8, 8.5, 9, 9.5, or about 10 nanoliters of liquid. In some instances, the reaction chamber comprises no more than 6, 6.5, 7, 7.5, 8, 8.5, 9, 10, 10.5, 11, 11.5, or no more than 12 nanoliters of buffer. In some instances, the reaction chamber comprises 0.5-10, 0.5-7, 0.5-5, 0.5-3, 1-4, or 2-4 nanoliters of buffer. In some instances the buffer pH is about 5.5, 6, 6.5, 7, 7.5, 8, or about 8.5. In some instances the buffer pH is 5.5-8.5, 5.5-8, 6-8, 6.5-7.5, or 7-8. In some instances, the reaction chamber comprises primers used for a PTA reaction, such as reversible or irreversible primers. In some instances, the temperature of the reaction chamber is 12-30, 10-30, 15-30, 15-25, 17-23, or 20-30 degrees C. In some instances, the temperature of the reaction chamber is about 15, 17, 20, 22, 25, 27, or about 30 degrees C. In some instances, one or more reagents configured for primary template-directed amplification (e.g., dNTPs, terminators, polymerases, or other component) are pre-spotted in the chamber and optionally dried prior to addition of droplets to the device. In some instances, the temperature of the reaction chamber starts at a first temperature at the beginning of PTA and is later cooled to a second temperature after the PTA reaction completes. In some instances, the temperature of the reaction chamber starts at 30 C at the beginning of PTA and is cooled to 12 C after the PTA reaction completes. In some instances, the PTA reaction is measured in real-time using a dye. In some instances, the dye is an intercalating dye. In some instances, the residence time for a sample in the reaction chamber is no more than 12, 10, 8, 6, 4, 3, or no more than 2 hours. In some instances, the residence time for a sample in the reaction chamber is about 12, 10, 8, 6, 4, 3, or about 2 hours. In some instances, the residence time for a sample in the reaction chamber is 2-12 hours, 2-10 hours, 2-8 hours, 1-8 hours, 4-10 hours, or 6-12 hours. In some instances, the residence time for a sample in the reaction chamber is determined based on the amount of amplification product produced. In some instances, the residence time for a sample in the reaction chamber is such that about 50, 60, 75, 80, 90, 100, 110, 125, 150, 200, or 500 ng of amplification product is produced. In some instances, the residence time for a sample in the reaction chamber is such that about 50-500, 50-200, 75-125, 50-100, 75-150, 100-250, 100-500, or 200-500 ng of amplification product is produced.

In a sixth step, with the external valve closed, the droplet is moved into an ERAT (end repair and A-tailing) chamber. In some instances, the ERAT chamber comprises about 40, 45, 50, 55, 60, 65, 70, 75, 80, 85, 90, or about 95 nanoliters of liquid. In some instances, the ERAT chamber comprises no more than 40, 45, 50, 55 60, 65, 70, 75, 80, 85, 90, or no more than 95 nanoliters of buffer. In some instances, the ERAT chamber comprises 40-100, 40-90, 50-70, 55-80, 60-100 or 80-120 nanoliters of buffer. In some instances the buffer pH is about 5.5, 6, 6.5, 7, 7.5, 8, or about 8.5. In some instances the buffer pH is 5.5-8.5, 5.5-8, 6-8, 6.5-7.5, or 7-8. In some instances, the temperature of the ERAT chamber is 10-72, 15-80, 10-50, 5-90, or 10-60 degrees C. In some instances, the temperature of the ERAT chamber is about 10, 20, 30, 40, 50, 60, 70, 80, or about 90 degrees C. In some instances, the temperature of the reaction chamber starts at a first temperature at the beginning of the ERAT reaction and is later cooled to a second temperature after the ERAT reaction completes. In some instances, the temperature of the reaction chamber starts at 12 C at the beginning of the ERAT reaction and is heated to 72 degrees C. after the ERAT reaction completes. In some instances, reagents for ERAT (e.g., polymerase, kinase, ATP, dNTPs, or other reagents) are pre-spotted in the chamber and optionally dried prior to addition of droplets to the device. In some instances, the residence time for a sample in the ERAT chamber is no more than 60 min, 50 min, 40 min, 30 min, 20 min, 15 min, 10 min, 5 min, or no more than 3 min. In some instances, the residence time for a sample in the ERAT chamber is 5-40, 5-30, 5-20, 5-60, 10-60, 10-50, or 20-40 minutes.

In a seventh step, the droplet is moved into a ligation chamber. In some instances, sequencing adapters are ligated to the end-repaired nucleic acids in the ligation chamber to generate an adapter-ligated library. In some instances, adapters comprise one or more sample barcodes uniquely identifying the source of a sample, or uniquely identifying each molecule in the sample. In some instances, the ligation chamber comprises about 80, 90, 100, 120, 130, 140, 150, 160, or about 170 nanoliters of liquid. In some instances, the ligation chamber comprises no more than 80, 90, 100, 120, 130, 140, 150, 160, or no more than 170 nanoliters of buffer. In some instances, the ligation chamber comprises 40-200, 60-200, 100-130, 80-160, 100-200 or 80-200 nanoliters of buffer. In some instances the buffer pH is about 5.5, 6, 6.5, 7, 7.5, 8, or about 8.5. In some instances the buffer pH is 5.5-8.5, 5.5-8, 6-8, 6.5-7.5, or 7-8. In some instances, the temperature of the ligation chamber is 10-72, 15-80, 10-50, 5-90, or 10-60 degrees C. In some instances, the temperature of the ligation chamber is about 5, 10, 15, 20, 25, 30, or about 40 degrees C. In some instances, reagents for ligation (e.g., ligase, adapters, or other reagents) are pre-spotted in the chamber and optionally dried prior to addition of droplets to the device. In some instances, adapters are pre-spotted in the ligation chamber at a concentration of 100 μM to 1 mM concentration and later dried. In some instances, the amount of adapters in the ligation chamber is 0.1-5, 0.2-2, 0.5-2, 1-2, or 1-3 picomoles. In some instances, adapters are pre-spotted in the ligation chamber in about 1, 2, 3, 4, 5, 6, 7, 8, or about 9 spots.

Methods described herein may comprise additional steps after ligation. In some instances, the adapter-ligated library is further amplified. In some instances, the adapter-ligated library (with or without amplification) is sequenced. In some instances, samples processed through devices described herein are pooled after the moving through the last chamber. In some instances, at least 5, 10, 20, 50, 75, 100, 200, 500 or more than 500 samples are pooled. In some instances, the total number of samples pooled from each device comprises at least 1000, 2000, 5000, 7000, or at least 10,000 cells.

Further described herein are methods for reducing reaction volumes of one or more steps described herein. In some instances, methods described herein comprise lowering the volume of the PTA reaction. In some instances, methods comprise zero-volume cell sorting. In some instances, cells are sorted and delivered to a chamber described herein in no more than 25, 50, 75, 100, 125, 150, 200, or no more than 250 nanoliters. In some instances, cells are sorted and delivered to a chamber described herein in 100-500, 50-500, 150-250, 200-500, or 75-150 nanoliters. In some instances, cell buffer is omitted from the PTA reaction. In some instances, cell buffer is omitted from the PTA reaction of single cells. In some instances, the total volume of a PTA reaction is no more than 1, 0.5, 0.2, 0.1, 0.05, or no more than 0.01 nanoliters. In some instances, the total volume of a PTA reaction is 5-500, 5-250, 10-250, 5-50, 5-15, or 5-25 nanoliters. In some instances, the total volume of a PTA reaction is no more than 200, 100, 50, 25, 10, or no more than 5 nanoliters.

Further described herein are methods for measuring PTA in real-time. In some instances, a dye is used to monitor the extent of a PTA reaction. In some instances, the dye comprises an intercalating dye. In some instances, the dye is Eva Green, SYBR green, SYTO dyes (e.g., 13, 16, 80, or 82), BRYT Green, or other dye. In some instances, the dye is Eva Green. In some instances, a molecular beacon dye is used. In some instances, the dye comprises a FRET pair. In some instances, the dye comprises a fluorophore and a quencher. In some instances, a dye comprises FAM, SUN, Hex, Cy3, Texas Red, or Cy5 fluorophore. In some instances, a dye comprises a ZEN/Iowa Black, TAO, or other quencher.

Further described herein are methods which allow reduced processing times. In some instances, a method described herein takes 5-12, 5-10, 3-12, 4-15, 4-10, 6-8, or 6-12 hours from sample addition to sample pooling or sequencing.

Array devices may be used for high-throughput cell analysis methods. In some instances, an array device comprising microwells is loaded with a sample. In some instances, the sample comprises cells, beads, droplets, or other sample type. In some instances, arrays are loaded with samples by diffusion, centrifugation, or other loading method. In some instances, the array is loaded to about 1%, 5%, 7%, 10%, 15%, 20%, 30%, 40%, 50%, 75%, or about 90% occupancy. In some instances, the array is loaded to at least 1%, 5%, 7%, 10%, 15%, 20%, 30%, 40%, 50%, 75%, or at least 90% occupancy. In some instances, the array is loaded to no more than 1%, 5%, 7%, 10%, 15%, 20%, 30%, 40%, 50%, 75%, or no more than 90% occupancy. In some instances, the PTA reaction is performed using arrays according to one or more of the steps in workflows shown in FIGS. 15B-15C. In some instances, a method comprises one or more of: capturing samples in microwells; drying samples in each microwell after loading; contacting the sample with a DNAse/RNAse cocktail; adding one or more of a lysis reagent, neutralization reagent, PTA reaction mixture, ERAT reagent, bead (comprising sequencing adapters), and ligation mix (with or without EDTA). In some instances reagents are added and dried sequentially. In some instances each microwell is dried under vacuum then refilled with next reagent under vacuum or by centrifugation. In some instances barcodes are added to nucleic acids obtained from each sample (e.g., cell). In some instances barcodes are either added at the priming step with beads or at the ligation step with indexes. In some instances, libraries generated using arrays are subjected to one or more of pooling, purifying, amplifying, and sequencing.

### Single Cell Multiomics

Described herein are methods, devices, and compositions for high-throughput analysis of single cells. Analysis of cells in bulk provides general information about the cell population, but often is unable to detect low-frequency mutants over the background. Such mutants may comprise important properties such as drug resistance or mutations associated with cancer. In some instances, DNA, RNA, and/or proteins from the same single cell are analyzed in parallel, using the devices described herein. The analysis may include identification of epigenetic post-translational (e.g., glycosylation, phosphorylation, acetylation, ubiquination, histone modification) and/or post-transcriptional (e.g., methylation, hydroxymethylation) modifications. Such methods may comprise “Primary Template-Directed Amplification” (PTA) to obtain libraries of nucleic acids for sequencing. In some instances PTA is combined with additional steps or methods such as RT-PCR or proteome/protein quantification techniques (e.g., mass spectrometry, antibody staining, etc.). In some instances, various components of a cell are physically or spatially separated from each other during individual analysis steps. In some instances, proteins are first labeled with antibodies. In some instances, at least some of the antibodies comprise a tag or marker (e.g., nucleic acid/oligo tag, mass tag, or fluorescent, tag). In some instances, a portion of the antibodies comprise an oligo tag. In some instances, a portion of the antibodies comprise a fluorescent marker. In some instances antibodies are labeled by two or more tags or markers. In some instances, a portion of the antibodies are sorted based on fluorescent markers. After RT-PCR, first strand mRNA products are generated and then removed for analysis. Libraries are then generated from RT-PCR products and barcodes present on protein-specific antibodies, which are subsequently sequenced. In parallel, genomic DNA from the same cell is subjected to PTA, a library generated, and sequenced. Sequencing results from the genome, proteome, and transcriptome are in some instances pooled using bioinformatics methods. Methods described herein in some instances comprise any combination of labeling, cell sorting, affinity separation/purification, lysing of specific cell components (e.g., outer membrane, nucleus, etc.), RNA amplification, DNA amplification (e.g., PTA), or other step associated with protein, RNA, or DNA isolation or analysis. In some instances, methods described herein comprise one or more enrichment steps, such as exome enrichment.

Described herein is a first method of single cell analysis comprising analysis of RNA and DNA from a single cell. In some instances, the method comprises isolation of single cells, lysis of single cells, and reverse transcription (RT). In some instances, reverse transcription is carried out with template switching oligonucleotides (TSOs). In some instances, TSOs comprise a molecular TAG such as biotin, which allows subsequent pull-down of cDNA RT products, and PCR amplification of RT products to generate a cDNA library. Alternatively or in combination, centrifugation is used to separate RNA in the supernatant from cDNA in the cell pellet. Remaining cDNA is in some instances fragmented and removed with UDG (uracil DNA glycosylase), and alkaline lysis is used to degrade RNA and denature the genome. After neutralization, addition of primers and PTA, amplification products are in some instances purified on SPRI (solid phase reversible immobilization) beads, and ligated to adapters to generate a gDNA library.

Described herein is a second method of single cell analysis comprising analysis of RNA and DNA from a single cell. In some instances, the method comprises isolation of single cells, lysis of single cells, and reverse transcription (RT). In some instances, reverse transcription is carried out with template switching oligonucleotides (TSOs). In some instances, TSOs comprise a molecular TAG such as biotin, which allows subsequent pull-down of cDNA RT products, and PCR amplification of RT products to generate a cDNA library. In some instances, alkaline lysis is then used to degrade RNA and denature the genome. After neutralization, addition of random primers and PTA, amplification products are in some instances purified on SPRI (solid phase reversible immobilization) beads, and ligated to adapters to generate a gDNA library. RT products are in some instances isolated by pulldown, such as a pulldown with streptavidin beads.

Described herein is a third method of single cell analysis comprising analysis of RNA and DNA from a single cell. In some instances, the method comprises isolation of single cells, lysis of single cells, and reverse transcription (RT). In some instances, reverse transcription is carried out with template switching oligonucleotides (TSOs) in the presence of terminator nucleotides. In some instances, TSOs comprise a molecular TAG such as biotin, which allows subsequent pull-down of cDNA RT products, and PCR amplification of RT products to generate a cDNA library. In some instances, alkaline lysis is then used to degrade RNA and denature the genome. After neutralization, addition of random primers and PTA, amplification products are in some instances purified on SPRI (solid phase reversible immobilization) beads, and ligated to adapters to generate a DNA library. RT products are in some instances isolated by pulldown, such as a pulldown with streptavidin beads.

Described herein is a fourth method of single cell analysis comprising analysis of RNA and DNA from a single cell. In some instances, the method comprises isolation of single cells, lysis of single cells, and reverse transcription (RT). In some instances, reverse transcription is carried out with template switching oligonucleotides (TSOs). In some instances, TSOs comprise a molecular TAG such as biotin, which allows subsequent pull-down of cDNA RT products, and PCR amplification of RT products to generate a cDNA library. In some instances, alkaline lysis is then used to degrade RNA and denature the genome. After neutralization, addition of random primers and PTA, amplification products are in some instances subjected to RNase and cDNA amplification using blocked and labeled primers. gDNA is purified on SPRI (solid phase reversible immobilization) beads, and ligated to adapters to generate a gDNA library. RT products are in some instances are isolated by pulldown, such as a pulldown with streptavidin beads.

Described herein is a fifth method of single cell analysis comprising analysis of RNA and DNA from a single cell. A population of cells is contacted with an antibody library, wherein antibodies are labeled. In some instances, antibodies are labeled with either fluorescent labels, nucleic acid barcodes, or both. Labeled antibodies bind to at least one cell in the population, and such cells are sorted, placing one cell per container (e.g., a tube, vial, microwell, etc.). In some instances, the container comprises a solvent. In some instances, a region of a surface of a container is coated with a capture moiety. In some instances, the capture moiety is a small molecule, an antibody, a protein, or other agent capable of binding to one or more cells, organelles, or other cell component. In some instances, at least one cell, or a single cell, or component thereof, binds to a region of the container surface. In some instances, a nucleus binds to the region of the container. In some instances, the outer membrane of the cell is lysed, releasing mRNA into a solution in the container. In some instances, the nucleus of the cell containing genomic DNA is bound to a region of the container surface. Next, RT is often performed using the mRNA in solution as a template to generate cDNA. In some instances, template switching primers comprise from 5′ to 3′ a TSS region (transcription start site), an anchor region, a RNA BC region, and a poly dT tail. In some instances, the poly dT tail binds to poly A tail of one or more mRNAs. In some instances, template switching primers comprise from 3′ to 5′ a TSS region, an anchor region, and a poly G region. In some instances, the poly G region comprises riboG. In some instances the poly G region binds to a poly C region on an mRNA transcript. In some instances, riboG was added to the mRNA transcripts by a terminal transferase. After removal of RT PCR products for subsequent sequencing, any remaining RNA in the cell is removed by UNG. The nucleus is then lysed, and the released genomic DNA is subjected to the PTA method using random primers with an isothermal polymerase. In some instances, primers are 6-9 bases in length. In some instances, PTA generates genomic amplicons of 100-5000, 200-5000, 500-2000, 500-2500, 1000-3000, or 300-3000 bases in length. In some instances, PTA generates genomic amplicons with an average length of 100-5000, 200-5000, 500-2000, 500-2500, 1000-3000, or 300-3000 bases. In some instances, PTA generates genomic amplicons of 250-1500 bases in length. In some instances, the methods described herein generate a short fragment cDNA pool with about 500, about 750, about 1000, about 5000, or about 10,000 fold amplification. In some instances, the methods described herein generate a short fragment cDNA pool with 500-5000, 750-1500, or 250-10,000 fold amplification. PTA products are optionally subjected to additional amplification and sequenced.

Additional devices and methods may be used for analysis of single cells. In some instances, cell printing is used in combination with PTA. In some instances, cell printing comprises use of a dot matrix printer. In some instances, cell printers comprise D100 single cell dispenser. In some instances, cell printers comprise D300 digital reagent dispenser. In some instances, cell printing is combined with PTA for drug combination studies, biochemical assays, cell-based assays, DMPK, assay development, secondary screening, synthetic biology, spotting, qPCR assay set up, enzyme profiling, antibody therapies, and/or SAR.

### Sample Preparation and Isolation of Single Cells

Methods described herein may require isolation of single cells for analysis. Any method of single cell isolation may be used with PTA, such as mouth pipetting, micro pipetting, flow cytometry/FACS, microfluidics, methods of sorting nuclei (tetraploid or other), or manual dilution. Such methods are aided by additional reagents and steps, for example, antibody-based enrichment (e.g., circulating tumor cells), other small-molecule or protein-based enrichment methods, or fluorescent labeling. In some instances, a method of multiomic analysis described herein comprises mechanical or enzymatic dissociate of cells from larger tissues.

### Preparation and Analysis of Cell Components

Methods of multiomic analysis comprising PTA described herein may comprise one or more methods of processing cell components such as DNA, RNA, and/or proteins. In some instances, the nucleus (comprising genomic DNA) is physically separated from the cytosol (comprising mRNA), followed by a membrane-selective lysis buffer to dissolve the membrane but keep the nucleus intact. The cytosol is then separated from the nucleus using methods including micro pipetting, centrifugation, or anti-body conjugated magnetic microbeads. In another instance, an oligo-dT primer coated magnetic bead binds polyadenylated mRNA for separation from DNA. In another instance, DNA and RNA are preamplified simultaneously, and then separated for analysis. In another instance, a single cell is split into two equal pieces, with mRNA from one half processed, and genomic DNA from the other half processed.

Use of the PTA method (e.g., two-step method) may lead to benefits in sequencing metrics. In some instances, sequencing metrics are benchmarked with a known cell line, such as NA12878. In some instances, sequencing results in an allelic balance of at least 0.5, 0.6, 0.7, 0.75 0.8, 0.85, 0.9, or at least 0.95. In some instances, sequencing results in a 1× coverage of at least 0.8, 0.85, 0.9, 0.92, 0.94, 0.95 0.96, 0.97, 0.98, 0.99, or at least 0.995. In some instances, sequencing results in a precision of at least 0.8, 0.85, 0.9, 0.92, 0.94, 0.95 0.96, 0.97, 0.98, 0.99, or at least 0.995. In some instances, sequencing results in an SNV sensitivity of at least 0.6, 0.7, 0.75, 0.8, 0.85, 0.9, 0.92, 0.94, 0.95 0.96, 0.97, 0.98, 0.99, or at least 0.995.

### Multiomics

Methods described herein (e.g., PTA) may be used as a replacement for any number of other known methods in the art which are used for single cell sequencing (multiomics or the like). PTA may substitute genomic DNA sequencing methods such as MDA, PicoPlex, DOP-PCR, MALBAC, or target-specific amplifications. In some instances, PTA replaces the standard genomic DNA sequencing method in a multiomics method including DR-seq (Dey et al., 2015), G&T seq (MacAulay et al., 2015), scMT-seq (Hu et al., 2016), sc-GEM (Cheow et al., 2016), scTrio-seq (Hou et al., 2016), simultaneous multiplexed measurement of RNA and proteins (Darmanis et al., 2016), scCOOL-seq (Guo et al., 2017), CITE-seq (Stoeckius et al., 2017), REAP-seq (Peterson et al., 2017), scNMT-seq (Clark et al., 2018), or SIDR-seq (Han et al., 2018). In some instances, a method described herein comprises PTA and a method of polyadenylated mRNA transcripts. In some instances, a method described herein comprises PTA and a method of non-polyadenylated mRNA transcripts. In some instances, a method described herein comprises PTA and a method of total (polyadenylated and non-polyadenylated) mRNA transcripts.

In some instances, PTA is combined with a standard RNA sequencing method to obtain genome and transcriptome data. In some instances, a multiomics method described herein comprises PTA and one of the following: Drop-seq (Macosko, et al. 2015), mRNA-seq (Tang et al., 2009), InDrop (Klein et al., 2015), MARS-seq (Jaitin et al., 2014), Smart-seq2 (Hashimshony, et al., 2012; Fish et al., 2016), CEL-seq (Jaitin et al., 2014), STRT-seq (Islam, et al., 2011), Quartz-seq (Sasagawa et al., 2013), CEL-seq2 (Hashimshony, et al. 2016), cytoSeq (Fan et al., 2015), SuPeR-seq (Fan et al., 2011), RamDA-seq (Hayashi, et al. 2018), MATQ-seq (Sheng et al., 2017), or SMARTer (Verboom et al., 2019).

Various reaction conditions and mixes may be used for generating cDNA libraries for transcriptome analysis. In some instances, an RT reaction mix is used to generate a cDNA library. In some instances, the RT reaction mixture comprises a crowding reagent, at least one primer, a template switching oligonucleotide (TSO), a reverse transcriptase, and a dNTP mix. In some instances, an RT reaction mix comprises an RNAse inhibitor. In some instances an RT reaction mix comprises one or more surfactants. In some instances an RT reaction mix comprises Tween-20 and/or Triton-X. In some instances an RT reaction mix comprises Betaine. In some instances an RT reaction mix comprises one or more salts. In some instances an RT reaction mix comprises a magnesium salt (e.g., magnesium chloride) and/or tetramethylammonium chloride. In some instances an RT reaction mix comprises gelatin. In some instances an RT reaction mix comprises PEG (PEG1000, PEG2000, PEG4000, PEG6000, PEG8000, or PEG of other length).

Multiomic methods described herein may provide both genomic and RNA transcript information from a single cell (e.g., a combined or dual protocol). In some instances, genomic information from the single cell is obtained from the PTA method, and RNA transcript information is obtained from reverse transcription to generate a cDNA library. In some instances, a whole transcript method is used to obtain the cDNA library. In some instances, 3′ or 5′ end counting is used to obtain the cDNA library. In some instances, cDNA libraries are not obtained using UMIs. In some instances, a multiomic method provides RNA transcript information from the single cell for at least 500, 1000, 2000, 5000, 8000, 10,000, 12,000, or at least 15,000 genes. In some instances, a multiomic method provides RNA transcript information from the single cell for about 500, 1000, 2000, 5000, 8000, 10,000, 12,000, or about 15,000 genes. In some instances, a multiomic method provides RNA transcript information from the single cell for 100-12,000 1000-10,000, 2000-15,000, 5000-15,000, 10,000-20,000, 8000-15,000, or 10,000-15,000 genes. In some instances, a multiomic method provides genomic sequence information for at least 80%, 90%, 92%, 95%, 97%, 98%, or at least 99% of the genome of the single cell. In some instances, a multiomic method provides genomic sequence information for about 80%, 90%, 92%, 95%, 97%, 98%, or about 99% of the genome of the single cell.

Multiomic methods may comprise analysis of single cells from a population of cells. In some instances, at least 5, 10, 20, 50, 100, 200, 500, 1000, 2000, 5000, or at least 8000 cells are analyzed. In some instances, about 5, 10, 20, 50, 100, 200, 500, 1000, 2000, 5000, or about 8000 cells are analyzed. In some instances, 5-100, 10-100, 50-500, 100-500, 100-1000, 50-5000, 100-5000, 500-1000, 500-10000, 1000-10000, or 5000-20,000 cells are analyzed.

Multiomic methods may generate yields of genomic DNA from the PTA reaction based on the type of single cell. In some instances, the amount of DNA generated from a single cell is about 0.1, 1, 1.5, 2, 3, 5, or about 10 micrograms. In some instances, the amount of DNA generated from a single cell is about 0.1, 1, 1.5, 2, 3, 5, or about 10 femtograms. In some instances, the amount of DNA generated from a single cell is at least 0.1, 1, 1.5, 2, 3, 5, or at least 10 micrograms. In some instances, the amount of DNA generated from a single cell is at least 0.1, 1, 1.5, 2, 3, 5, or at least 10 femtograms. In some instances, the amount of DNA generated from a single cell is about 0.1-10, 1-10, 1.5-10, 2-20, 2-50, 1-3, or 0.5-3.5 micrograms. In some instances, the amount of DNA generated from a single cell is about 0.1-10, 1-10, 1.5-10, 2-20, 2-4, 1-3, or 0.5-4 femtograms.

### Methylome Analysis

Described herein are methods comprising PTA, wherein sites of methylated DNA in single cells are determined using the PTA method. In some instances, these methods further comprise parallel analysis of the transcriptome and/or proteome of the same cell. Methods of detecting methylated genomic bases include selective restriction with methylation-sensitive endonucleases, followed by processing with the PTA method. Sites cut by such enzymes are determined from sequencing, and methylated bases are identified. In another instance, bisulfite treatment of genomic DNA libraries converts unmethylated cytosines to uracil. Libraries are then in some instances amplified with methylation-specific primers which selectively anneal to methylated sequences. Alternatively, non-methylation-specific PCR is conducted, followed by one or more methods to discriminate between bisulfite-reacted bases, including direct pyrosequencing, MS-SnuPE, HRM, COBRA, MS-SSCA, or base-specific cleavage/MALDI-TOF. In some instances, genomic DNA samples are split for parallel analysis of the genome (or an enriched portion thereof) and methylome analysis. In some instances, analysis of the genome and methylome comprises enrichment of genomic fragments (e.g., exome, or other targets) or whole genome sequencing.

### Bioinformatics

The data obtained from single-cell analysis methods utilizing PTA described herein may be compiled into a database. Described herein are methods and systems of bioinformatic data integration. Data from the proteome, genome, transcriptome, methylome or other data is in some instances combined/integrated into a database and analyzed. Bioinformatic data integration methods and systems in some instances comprise one or more of protein detection (FACS and/or NGS), mRNA detection, and/or genome variance detection. In some instances, this data is correlated with a disease state or condition. In some instances, data from a plurality of single cells is compiled to describe properties of a larger cell population, such as cells from a specific sample, region, organism, or tissue. In some instances, protein data is acquired from fluorescently labeled antibodies which selectively bind to proteins on a cell. In some instances, a method of protein detection comprises grouping cells based on fluorescent markers and reporting sample location post-sorting. In some instances, a method of protein detection comprises detecting sample barcodes, detecting protein barcodes, comparing to designed sequences, and grouping cells based on barcode and copy number. In some instances, protein data is acquired from barcoded antibodies which selectively bind to proteins on a cell. In some instances, transcriptome data is acquired from sample and RNA specific barcodes. In some instances, a method of mRNA detection comprises detecting sample and RNA specific barcodes, aligning to genome, aligning to RefSeq/Encode, reporting Exon/Intro/Intergenic sequences, analyzing exon-exon junctions, grouping cells based on barcode and expression variance and clustering analysis of variance and top variable genes. In some instances, genomic data is acquired from sample and DNA specific barcodes. In some instances, a method of genome variance detection comprises detecting sample and DNA specific barcodes, aligning to the genome, determine genome recovery and SNV mapping rate, filtering reads on exon-exon junctions, generating variant call file (VCF), and clustering analysis of variance and top variable mutations.

### Mutations

In some instances, the methods (e.g., multiomic PTA) described herein result in higher detection sensitivity and/or lower rates of false positives for the detection of mutations. In some instances a mutation is a difference between an analyzed sequence (e.g., using the methods described herein) and a reference sequence. Reference sequences are in some instances obtained from other organisms, other individuals of the same or similar species, populations of organisms, or other areas of the same genome. In some instances, mutations are identified on a plasmid or chromosome. In some instances, a mutation is an SNV (single nucleotide variation), SNP (single nucleotide polymorphism), or CNV (copy number variation, or CNA/copy number aberration). In some instances, a mutation is base substitution, insertion, or deletion. In some instances, a mutation is a transition, transversion, nonsense mutation, silent mutation, synonymous or non-synonymous mutation, non-pathogenic mutation, missense mutation, or frameshift mutation (deletion or insertion). In some instances, PTA results in higher detection sensitivity and/or lower rates of false positives for the detection of mutations when compared to methods such as in-silico prediction, ChIP-seq, GUIDE-seq, circle-seq, HTGTS (High-Throughput Genome-Wide Translocation Sequencing), IDLV (integration-deficient lentivirus), Digenome-seq, FISH (fluorescence in situ hybridization), or DISCOVER-seq.

### Primary Template-Directed Amplification

Described herein are nucleic acid amplification methods, such as “Primary Template-Directed Amplification (PTA).” In some instances, PTA is combined with other analysis workflows for multiomic analysis. With the PTA method, amplicons are preferentially generated from the primary template (“direct copies”) using a polymerase (e.g., a strand displacing polymerase). Consequently, errors are propagated at a lower rate from daughter amplicons during subsequent amplifications compared to MDA. The result is an easily executed method that, unlike existing WGA protocols, can amplify low DNA input including the genomes of single cells with high coverage breadth and uniformity in an accurate and reproducible manner. In some instances, PTA enables kinetic control of an amplification reaction. In some instances, PTA results in a pseudo-linear amplification reaction (rather than exponential amplification). Moreover, the terminated amplification products can undergo direction ligation after removal of the terminators, allowing for the attachment of a cell barcode to the amplification primers so that products from all cells can be pooled after undergoing parallel amplification reactions. In some instances, template nucleic acids are not bound to a solid support. In some instances, direct copies of template nucleic acids are not bound to a solid support. In some instances, one or more primers are not bound to a solid support. In some instances, no primers are not bound to a solid support. In some instances, a primer is attached to a first solid support, and a template nucleic acid is attached to a second solid support, wherein the first and the second solid supports are not the same. In some instances, PTA is used to analyze single cells from a larger population of cells. In some instances, PTA is used to analyze more than one cell from a larger population of cells, or an entire population of cells.

Described herein are methods employing nucleic acid polymerases with strand displacement activity for amplification. In some instances, such polymerases comprise strand displacement activity and low error rate. In some instances, such polymerases comprise strand displacement activity and proofreading exonuclease activity, such as 3′->5′ proofreading activity. In some instances, nucleic acid polymerases are used in conjunction with other components such as reversible or irreversible terminators, or additional strand displacement factors. In some instances, the polymerase has strand displacement activity, but does not have exonuclease proofreading activity. For example, in some instances such polymerases include bacteriophage phi29 (@29) polymerase, which also has very low error rate that is the result of the 3′->5′ proofreading exonuclease activity (see, e.g., U.S. Pat. Nos. 5,198,543 and 5,001,050). In some instances, non-limiting examples of strand displacing nucleic acid polymerases include, e.g., genetically modified phi29 (@29) DNA polymerase, Klenow Fragment of DNA polymerase I (Jacobsen et al., Eur. J. Biochem. 45:623-627 (1974)), phage M2 DNA polymerase (Matsumoto et al., Gene 84:247 (1989)), phage phiPRD1 DNA polymerase (Jung et al., Proc. Natl. Acad. Sci. USA 84:8287 (1987); Zhu and Ito, Biochim. Biophys. Acta. 1219:267-276 (1994)), Bst DNA polymerase (e.g., Bst large fragment DNA polymerase (Exo (-) Bst; Aliotta et al., Genet. Anal. (Netherlands) 12:185-195 (1996)), exo (-) Bca DNA polymerase (Walker and Linn, Clinical Chemistry 42:1604-1608 (1996)), Bsu DNA polymerase, Vent_{R} DNA polymerase including Vent_{R} (exo-) DNA polymerase (Kong et al., J. Biol. Chem. 268:1965-1975 (1993)), Deep Vent DNA polymerase including Deep Vent (exo-) DNA polymerase, IsoPol DNA polymerase, DNA polymerase I, Therminator DNA polymerase, T5 DNA polymerase (Chatterjee et al., Gene 97:13-19 (1991)), Sequenase (U.S. Biochemicals), T7 DNA polymerase, T7-Sequenase, T7 gp5 DNA polymerase, PRDI DNA polymerase, T4 DNA polymerase (Kaboord and Benkovic, Curr. Biol. 5:149-157 (1995)). Additional strand displacing nucleic acid polymerases are also compatible with the methods described herein. The ability of a given polymerase to carry out strand displacement replication can be determined, for example, by using the polymerase in a strand displacement replication assay (e.g., as disclosed in U.S. Pat. No. 6,977,148). Such assays in some instances are performed at a temperature suitable for optimal activity for the enzyme being used, for example, 32° C. for phi29 DNA polymerase, from 46° C. to 64° C. for exo (-) Bst DNA polymerase, or from about 60° C. to 70° C. for an enzyme from a hyperthermophylic organism. Another useful assay for selecting a polymerase is the primer-block assay described in Kong et al., J. Biol. Chem. 268:1965-1975 (1993). The assay consists of a primer extension assay using an M13 ssDNA template in the presence or absence of an oligonucleotide that is hybridized upstream of the extending primer to block its progress. Other enzymes capable of displacement the blocking primer in this assay are in some instances useful for the disclosed method. In some instances, polymerases incorporate dNTPs and terminators at approximately equal rates. In some instances, the ratio of rates of incorporation for dNTPs and terminators for a polymerase described herein are about 1:1, about 1.5:1, about 2:1, about 3:1 about 4:1 about 5:1, about 10:1, about 20:1 about 50:1, about 100:1, about 200:1, about 500:1, or about 1000:1. In some instances, the ratio of rates of incorporation for dNTPs and terminators for a polymerase described herein are 1:1 to 1000:1, 2:1 to 500:1, 5:1 to 100:1, 10:1 to 1000:1, 100:1 to 1000:1, 500:1 to 2000:1, 50:1 to 1500:1, or 25:1 to 1000:1. In some instances, “hot” or “warm” start polymerases are used. In some instances, polymerases have substantially no polymerase activity until heated to at least 40 degrees C. In some instances, such polymerases allow for uniform control over amplification start times when multiple samples are processed.

A polynucleotide mixture used herein for PTA may comprise dNTPs. In some instances, dNTPs comprise one or more of dA, dT, dG, and dC. In some instances, the concentration of dNTPs is no more than 10, 8, 7, 5, 4, 3, 2, 1, 0.5, 0.2, 0.1, 0.05, or no more than 0.01 mM. In some instances, the concentration of dNTPs is 0.5-10, 0.5-5, 0.5-3, 0.5-2.5, 0.5-2, 0.5-1.5, 0.5-1, 0.1-5, 0.1-3, 0.1-3, 1-3, 0.5-2.5, or 1-2 mM. Such mixtures in some instances also comprise one or more terminators.

A polynucleotide mixture used herein for PTA may comprise terminators. In some instances, terminators comprise ddNTPs. In some instances, terminators comprise irreversible terminators. In some instances, irreversible terminators comprise alpha-thio dideoxynucleotides. In some instances, the concentration of terminators is no more than 1, 0.8, 0.7, 0.5, 0.4, 0.3, 0.2, 0.1, 0.05, 0.02, 0.01, 0.005, or no more than 0.001 mM. In some instances, the concentration of dNTPs is 0.05-1, 0.05-0.5, 0.05-0.3, 0.05-0.25, 0.05-0.2, 0.05-0.15, 0.05-0.1, 0.01-0.5, 0.01-0.3, 0.01-0.3, 0.1-0.3, 0.05-0.25, or 0.1-0.2 mM.

Described herein are methods of amplification wherein strand displacement can be facilitated through the use of a strand displacement factor, such as, e.g., helicase. Such factors are in some instances used in conjunction with additional amplification components, such as polymerases, terminators, or other component. In some instances, a strand displacement factor is used with a polymerase that does not have strand displacement activity. In some instances, a strand displacement factor is used with a polymerase having strand displacement activity. Without being bound by theory, strand displacement factors may increase the rate that smaller, double stranded amplicons are reprimed. In some instances, any DNA polymerase that can perform strand displacement replication in the presence of a strand displacement factor is suitable for use in the PTA method, even if the DNA polymerase does not perform strand displacement replication in the absence of such a factor. Strand displacement factors useful in strand displacement replication in some instances include (but are not limited to) BMRF1 polymerase accessory subunit (Tsurumi et al., J. Virology 67 (12): 7648-7653 (1993)), adenovirus DNA-binding protein (Zijderveld and van der Vliet, J. Virology 68 (2): 1158-1164 (1994)), herpes simplex viral protein ICP8 (Boehmer and Lehman, J. Virology 67 (2): 711-715 (1993); Skaliter and Lehman, Proc. Natl. Acad. Sci. USA 91 (22): 10665-10669 (1994)); single-stranded DNA binding proteins (SSB; Rigler and Romano, J. Biol. Chem. 270:8910-8919 (1995)); phage T4 gene 32 protein (Villemain and Giedroc, Biochemistry 35:14395-14404 (1996); T7 helicase-primase; T7 gp2.5 SSB protein; Tte-UvrD (from Thermoanaerobacter tengcongensis), calf thymus helicase (Siegel et al., J. Biol. Chem. 267:13629-13635 (1992)); bacterial SSB (e.g., E. coli SSB), Replication Protein A (RPA) in eukaryotes, human mitochondrial SSB (mtSSB), and recombinases, (e.g., Recombinase A (RecA) family proteins, T4 UvsX, T4 UvsY, Sak4 of Phage HK620, Rad51, Dmc1, or Radb). Combinations of factors that facilitate strand displacement and priming are also consistent with the methods described herein. For example, a helicase is used in conjunction with a polymerase. In some instances, the PTA method comprises use of a single-strand DNA binding protein (SSB, T4 gp32, or other single stranded DNA binding protein), a helicase, and a polymerase (e.g., SauDNA polymerase, Bsu polymerase, Bst2.0, GspM, GspM2.0, GspSSD, or other suitable polymerase). In some instances, reverse transcriptases are used in conjunction with the strand displacement factors described herein. In some instances, reverse transcriptases are used in conjunction with the strand displacement factors described herein. In some instances, amplification is conducted using a polymerase and a nicking enzyme (e.g., “NEAR”), such as those described in U.S. Pat. No. 9,617,586. In some instances, the nicking enzyme is Nt.BspQI, Nb.BbvCi, Nb.BsmI, Nb.BsrDI, Nb.BtsI, Nt.AlwI, Nt.BbvCI, Nt.BstNBI, Nt. CviPII, Nb.Bpu10I, or Nt.Bpu10I.

Described herein are amplification methods comprising use of terminator nucleotides, polymerases, and additional factors or conditions. For example, such factors are used in some instances to fragment the nucleic acid template(s) or amplicons during amplification. In some instances, such factors comprise endonucleases. In some instances, factors comprise transposases. In some instances, mechanical shearing is used to fragment nucleic acids during amplification. In some instances, nucleotides are added during amplification that may be fragmented through the addition of additional proteins or conditions. For example, uracil is incorporated into amplicons; treatment with uracil D-glycosylase fragments nucleic acids at uracil-containing positions. Additional systems for selective nucleic acid fragmentation are also in some instances employed, for example an engineered DNA glycosylase that cleaves modified cytosine-pyrene base pairs. (Kwon, et al. Chem Biol. 2003, 10 (4), 351)

Described herein are amplification methods comprising use of terminator nucleotides, which terminate nucleic acid replication thus decreasing the size of the amplification products. Such terminators are in some instances used in conjunction with polymerases, strand displacement factors, or other amplification components described herein. In some instances, terminator nucleotides reduce or lower the efficiency of nucleic acid replication. Such terminators in some instances reduce extension rates by at least 99.9%, 99%, 98%, 95%, 90%, 85%, 80%, 75%, 70%, or at least 65%. Such terminators in some instances reduce extension rates by 50%-90%, 60%-80%, 65%-90%, 70%-85%, 60%-90%, 70%-99%, 80%-99%, or 50%-80%. In some instances terminators reduce the average amplicon product length by at least 99.9%, 99%, 98%, 95%, 90%, 85%, 80%, 75%, 70%, or at least 65%. Terminators in some instances reduce the average amplicon length by 50%-90%, 60%-80%, 65%-90%, 70%-85%, 60%-90%, 70%-99%, 80%-99%, or 50%-80%. In some instances, amplicons comprising terminator nucleotides form loops or hairpins which reduce a polymerase's ability to use such amplicons as templates. Use of terminators in some instances slows the rate of amplification at initial amplification sites through the incorporation of terminator nucleotides (e.g., dideoxynucleotides that have been modified to make them exonuclease-resistant to terminate DNA extension), resulting in smaller amplification products. By producing smaller amplification products than the currently used methods (e.g., average length of 50-2000 nucleotides in length for PTA methods as compared to an average product length of >10,000 nucleotides for MDA methods) PTA amplification products in some instances undergo direct ligation of adapters without the need for fragmentation, allowing for efficient incorporation of cell barcodes and unique molecular identifiers (UMI).

Terminator nucleotides are present at various concentrations depending on factors such as polymerase, template, or other factors. For example, the amount of terminator nucleotides in some instances is expressed as a ratio of non-terminator nucleotides to terminator nucleotides in a method described herein. Such concentrations in some instances allow control of amplicon lengths. In some instances, the ratio of terminator to non-terminator nucleotides is modified for the amount of template present or the size of the template. In some instances, the ratio of ratio of terminator to non-terminator nucleotides is reduced for smaller samples sizes (e.g., femtogram to picogram range). In some instances, the ratio of non-terminator to terminator nucleotides is about 2:1, 5:1, 7:1, 10:1, 20:1, 50:1, 100:1, 200:1, 500:1, 1000:1, 2000:1, or 5000:1. In some instances the ratio of non-terminator to terminator nucleotides is 2:1-10:1, 5:1-20:1, 10:1-100:1, 20:1-200:1, 50:1-1000:1, 50:1-500:1, 75:1-150:1, or 100:1-500:1. In some instances, at least one of the nucleotides present during amplification using a method described herein is a terminator nucleotide. Each terminator need not be present at approximately the same concentration; in some instances, ratios of each terminator present in a method described herein are optimized for a particular set of reaction conditions, sample type, or polymerase. Without being bound by theory, each terminator may possess a different efficiency for incorporation into the growing polynucleotide chain of an amplicon, in response to pairing with the corresponding nucleotide on the template strand. For example, in some instances a terminator pairing with cytosine is present at about 3%, 5%, 10%, 15%, 20%, 25%, or 50% higher concentration than the average terminator concentration. In some instances a terminator pairing with thymine is present at about 3%, 5%, 10%, 15%, 20%, 25%, or 50% higher concentration than the average terminator concentration. In some instances a terminator pairing with guanine is present at about 3%, 5%, 10%, 15%, 20%, 25%, or 50% higher concentration than the average terminator concentration. In some instances a terminator pairing with adenine is present at about 3%, 5%, 10%, 15%, 20%, 25%, or 50% higher concentration than the average terminator concentration. In some instances a terminator pairing with uracil is present at about 3%, 5%, 10%, 15%, 20%, 25%, or 50% higher concentration than the average terminator concentration. Any nucleotide capable of terminating nucleic acid extension by a nucleic acid polymerase in some instances is used as a terminator nucleotide in the methods described herein. In some instances, a reversible terminator is used to terminate nucleic acid replication. In some instances, a non-reversible terminator is used to terminate nucleic acid replication. In some instances, non-limited examples of terminators include reversible and non-reversible nucleic acids and nucleic acid analogs, such as, e.g., 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, or any combination thereof. In one embodiment, terminator nucleotides are dideoxynucleotides. Other nucleotide modifications that terminate nucleic acid replication and may be suitable for practicing the invention include, without limitation, any modifications of the r group of the 3′ carbon of the deoxyribose such as inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. In some instances, terminators are polynucleotides comprising 1, 2, 3, 4, or more bases in length. In some instances, terminators do not comprise a detectable moiety or tag (e.g., mass tag, fluorescent tag, dye, radioactive atom, or other detectable moiety). In some instances, terminators do not comprise a chemical moiety allowing for attachment of a detectable moiety or tag (e.g., “click” azide/alkyne, conjugate addition partner, or other chemical handle for attachment of a tag). In some instances, all terminator nucleotides comprise the same modification that reduces amplification to at region (e.g., the sugar moiety, base moiety, or phosphate moiety) of the nucleotide. In some instances, at least one terminator has a different modification that reduces amplification. In some instances, all terminators have a substantially similar fluorescent excitation or emission wavelengths. In some instances, terminators without modification to the phosphate group are used with polymerases that do not have exonuclease proofreading activity. Terminators, when used with polymerases which have 3′->5′ proofreading exonuclease activity (such as, e.g., phi29) that can remove the terminator nucleotide, are in some instances further modified to make them exonuclease-resistant. For example, dideoxynucleotides are modified with an alpha-thio group that creates a phosphorothioate linkage which makes these nucleotides resistant to the 3′->5′ proofreading exonuclease activity of nucleic acid polymerases. Such modifications in some instances reduce the exonuclease proofreading activity of polymerases by at least 99.5%, 99%, 98%, 95%, 90%, or at least 85%. Non-limiting examples of other terminator nucleotide modifications providing resistance to the 3′->5′ exonuclease activity include in some instances: nucleotides with modification to the alpha group, such as alpha-thio dideoxynucleotides creating a phosphorothioate bond, C3 spacer nucleotides, locked nucleic acids (LNA), inverted nucleic acids, 2′ Fluoro bases, 3′ phosphorylation, 2′-O-Methyl modifications (or other 2′-O-alkyl modification), propyne-modified bases (e.g., deoxycytosine, deoxyuridine), L-DNA nucleotides, L-RNA nucleotides, nucleotides with inverted linkages (e.g., 5′-5′ or 3′-3′), 5′ inverted bases (e.g., 5′ inverted 2′,3′-dideoxy dT), methylphosphonate backbones, and trans nucleic acids. In some instances, nucleotides with modification include base-modified nucleic acids comprising free 3′ OH groups (e.g., 2-nitrobenzyl alkylated HOMedU triphosphates, bases comprising modification with large chemical groups, such as solid supports or other large moiety). In some instances, a polymerase with strand displacement activity but without 3′->5′exonuclease proofreading activity is used with terminator nucleotides with or without modifications to make them exonuclease resistant. Such nucleic acid polymerases include, without limitation, Bst DNA polymerase, Bsu DNA polymerase, Deep Vent (exo-) DNA polymerase, Klenow Fragment (exo-) DNA polymerase, Therminator DNA polymerase, and Vent_{R} (exo-).

### Primers and Amplicon Libraries

Described herein are amplicon libraries resulting from amplification of at least one target nucleic acid molecule. Such libraries are in some instances generated using the methods described herein, such as those using terminators. Such methods comprise use of strand displacement polymerases or factors, terminator nucleotides (reversible or irreversible), or other features and embodiments described herein. In some instances, reversible terminators are capable of removal by an exonuclease (e.g., or polymerase having exonuclease activity). In some instances, irreversible terminators are not capable of substantial removal by an exonuclease (e.g., or polymerase having exonuclease activity). In some instances, amplicon libraries generated by use of terminators described herein are further amplified in a subsequent amplification reaction (e.g., PCR). In some instances, subsequent amplification reactions do not comprise terminators. In some instances, amplicon libraries comprise polynucleotides, wherein at least 50%, 60%, 70%, 80%, 90%, 95%, or at least 98% of the polynucleotides comprise at least one terminator nucleotide. In some instances, the amplicon library comprises the target nucleic acid molecule from which the amplicon library was derived. The amplicon library comprises a plurality of polynucleotides, wherein at least some of the polynucleotides are direct copies (e.g., replicated directly from a target nucleic acid molecule, such as genomic DNA, RNA, or other target nucleic acid). For example, at least 5%, 10%, 20%, 30%, 40%, 50%, 60%, 70%, 80%, 90%, 95% or more than 95% of the amplicon polynucleotides are direct copies of the at least one target nucleic acid molecule. In some instances, at least 5% of the amplicon polynucleotides are direct copies of the at least one target nucleic acid molecule. In some instances, at least 10% of the amplicon polynucleotides are direct copies of the at least one target nucleic acid molecule. In some instances, at least 15% of the amplicon polynucleotides are direct copies of the at least one target nucleic acid molecule. In some instances, at least 20% of the amplicon polynucleotides are direct copies of the at least one target nucleic acid molecule. In some instances, at least 50% of the amplicon polynucleotides are direct copies of the at least one target nucleic acid molecule. In some instances, 3%-5%, 3-10%, 5%-10%, 10%-20%, 20%-30%, 30%-40%, 5%-30%, 10%-50%, or 15%-75% of the amplicon polynucleotides are direct copies of the at least one target nucleic acid molecule. In some instances, at least some of the polynucleotides are direct copies of the target nucleic acid molecule, or daughter (a first copy of the target nucleic acid) progeny. For example, at least 5%, 10%, 20%, 30%, 40%, 50%, 60%, 70%, 80%, 90%, 95% or more than 95% of the amplicon polynucleotides are direct copies of the at least one target nucleic acid molecule or daughter progeny. In some instances, at least 5% of the amplicon polynucleotides are direct copies of the at least one target nucleic acid molecule or daughter progeny. In some instances, at least 10% of the amplicon polynucleotides are direct copies of the at least one target nucleic acid molecule or daughter progeny. In some instances, at least 20% of the amplicon polynucleotides are direct copies of the at least one target nucleic acid molecule or daughter progeny. In some instances, at least 30% of the amplicon polynucleotides are direct copies of the at least one target nucleic acid molecule or daughter progeny. In some instances, 3%-5%, 3%-10%, 5%-10%, 10%-20%, 20%-30%, 30%-40%, 5%-30%, 10%-50%, or 15%-75% of the amplicon polynucleotides are direct copies of the at least one target nucleic acid molecule or daughter progeny. In some instances, direct copies of the target nucleic acid are 50-2500, 75-2000, 50-2000, 25-1000, 50-1000, 500-2000, or 50-2000 bases in length. In some instances, daughter progeny are 1000-5000, 2000-5000, 1000-10,000, 2000-5000, 1500-5000, 3000-7000, or 2000-7000 bases in length. In some instances, the average length of PTA amplification products is 25-3000 nucleotides in length, 50-2500, 75-2000, 50-2000, 25-1000, 50-1000, 500-2000, or 50-2000 bases in length. In some instance, amplicons generated from PTA are no more than 5000, 4000, 3000, 2000, 1700, 1500, 1200, 1000, 700, 500, or no more than 300 bases in length. In some instance, amplicons generated from PTA are 1000-5000, 1000-3000, 200-2000, 200-4000, 500-2000, 750-2500, or 1000-2000 bases in length. Amplicon libraries generated using the methods described herein in some instances comprise at least 1000, 2000, 5000, 10,000, 100,000, 200,000, 500,000 or more than 500,000 amplicons comprising unique sequences. In some instances, the library comprises at least 100, 200, 300, 400, 500, 600, 700, 800, 900, 1000, 1100, 1200, 1300, 1400, 1500, 2000, 2500, 3000, or at least 3500 amplicons. In some instances, at least 5%, 10%, 15%, 20%, 25%, 30% or more than 30% of amplicon polynucleotides having a length of less than 1000 bases are direct copies of the at least one target nucleic acid molecule. In some instances, at least 5%, 10%, 15%, 20%, 25%, 30% or more than 30% of amplicon polynucleotides having a length of no more than 2000 bases are direct copies of the at least one target nucleic acid molecule. In some instances, at least 5%, 10%, 15%, 20%, 25%, 30% or more than 30% of amplicon polynucleotides having a length of 3000-5000 bases are direct copies of the at least one target nucleic acid molecule. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are no more than 700-1200 bases in length. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are 700-1200 bases in length, and the daughter amplicons are 2500-6000 bases in length. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule or daughter amplicons. The number of direct copies may be controlled in some instances by the number of amplification cycles. In some instances, no more than 30, 25, 20, 15, 13, 11, 10, 9, 8, 7, 6, 5, 4, or 3 cycles are used to generate copies of the target nucleic acid molecule. In some instances, about 30, 25, 20, 15, 13, 11, 10, 9, 8, 7, 6, 5, 4, or about 3 cycles are used to generate copies of the target nucleic acid molecule. In some instances, 3, 4, 5, 6, 7, or 8 cycles are used to generate copies of the target nucleic acid molecule. In some instances, 2-4, 2-5, 2-7, 2-8, 2-10, 2-15, 3-5, 3-10, 3-15, 4-10, 4-15, 5-10 or 5-15 cycles are used to generate copies of the target nucleic acid molecule. Amplicon libraries generated using the methods described herein are in some instances subjected to additional steps, such as adapter ligation and further amplification. In some instances, such additional steps precede a sequencing step. In some instances, the cycles are PCR cycles. In some instances, the cycles represent annealing, extension, and denaturation. In some instances, the cycles represent annealing, extension, and denaturation which occur under isothermal or essentially isothermal conditions.

Methods described herein may additionally comprise one or more enrichment or purification steps. In some instances, one or more polynucleotides (such as cDNA, PTA amplicons, or other polynucleotide) are enriched during a method described herein. In some instances, polynucleotide probes are used to capture one or more polynucleotides. In some instances, probes are configured to capture one or more genomic exons. In some instances, a library of probes comprises at least 1000, 2000, 5000, 10,000, 50,000, 100,000, 200,000, 500,000, or more than 1 million different sequences. In some instances, a library of probes comprises sequences capable of binding to at least 10, 20, 50, 100, 200, 500, 1000, 2000, 5000, 10,000 or more than 10,000 genes. In some instances, probes comprise a moiety for capture by a solid support, such as biotin. In some instances, an enrichment step occurs after a PTA step. In some instances, an enrichment step occurs before a PTA step. In some instances, probes are configured to bind genomic DNA libraries. In some instances, probes are configured to bind cDNA libraries.

Amplicon libraries of polynucleotides generated from the PTA methods and compositions (terminators, polymerases, etc.) described herein in some instances have increased uniformity. Uniformity, in some instances, is described using a Lorenz curve, or other such method. Such increases in some instances lead to lower sequencing reads needed for the desired coverage of a target nucleic acid molecule (e.g., genomic DNA, RNA, or other target nucleic acid molecule). For example, no more than 50% of a cumulative fraction of polynucleotides comprises sequences of at least 80% of a cumulative fraction of sequences of the target nucleic acid molecule. In some instances, no more than 50% of a cumulative fraction of polynucleotides comprises sequences of at least 60% of a cumulative fraction of sequences of the target nucleic acid molecule. In some instances, no more than 50% of a cumulative fraction of polynucleotides comprises sequences of at least 70% of a cumulative fraction of sequences of the target nucleic acid molecule. In some instances, no more than 50% of a cumulative fraction of polynucleotides comprises sequences of at least 90% of a cumulative fraction of sequences of the target nucleic acid molecule. In some instances, uniformity is described using a Gini index (wherein an index of 0 represents perfect equality of the library and an index of 1 represents perfect inequality). In some instances, amplicon libraries described herein have a Gini index of no more than 0.55, 0.50, 0.45, 0.40, or 0.30. In some instances, amplicon libraries described herein have a Gini index of no more than 0.50. In some instances, amplicon libraries described herein have a Gini index of no more than 0.40. Such uniformity metrics in some instances are dependent on the number of reads obtained. For example, no more than 100 million, 200 million, 300 million, 400 million, or no more than 500 million reads are obtained. In some instances, the read length is about 50,75, 100, 125, 150, 175, 200, 225, or about 250 bases in length. In some instances, uniformity metrics are dependent on the depth of coverage of a target nucleic acid. For example, the average depth of coverage is about 10×, 15×, 20×, 25×, or about 30×. In some instances, the average depth of coverage is 10-30×, 20-50×, 5-40×, 20-60×, 5-20×, or 10-20×. In some instances, amplicon libraries described herein have a Gini index of no more than 0.55, wherein about 300 million reads was obtained. In some instances, amplicon libraries described herein have a Gini index of no more than 0.50, wherein about 300 million reads was obtained. In some instances, amplicon libraries described herein have a Gini index of no more than 0.45, wherein about 300 million reads was obtained. In some instances, amplicon libraries described herein have a Gini index of no more than 0.55, wherein no more than 300 million reads was obtained. In some instances, amplicon libraries described herein have a Gini index of no more than 0.50, wherein no more than 300 million reads was obtained. In some instances, amplicon libraries described herein have a Gini index of no more than 0.45, wherein no more than 300 million reads was obtained. In some instances, amplicon libraries described herein have a Gini index of no more than 0.55, wherein the average depth of sequencing coverage is about 15×. In some instances, amplicon libraries described herein have a Gini index of no more than 0.50, wherein the average depth of sequencing coverage is about 15×. In some instances, amplicon libraries described herein have a Gini index of no more than 0.45, wherein the average depth of sequencing coverage is about 15×. In some instances, amplicon libraries described herein have a Gini index of no more than 0.55, wherein the average depth of sequencing coverage is at least 15×. In some instances, amplicon libraries described herein have a Gini index of no more than 0.50, wherein the average depth of sequencing coverage is at least 15×. In some instances, amplicon libraries described herein have a Gini index of no more than 0.45, wherein the average depth of sequencing coverage is at least 15×. In some instances, amplicon libraries described herein have a Gini index of no more than 0.55, wherein the average depth of sequencing coverage is no more than 15×. In some instances, amplicon libraries described herein have a Gini index of no more than 0.50, wherein the average depth of sequencing coverage is no more than 15×. In some instances, amplicon libraries described herein have a Gini index of no more than 0.45, wherein the average depth of sequencing coverage is no more than 15×. Uniform amplicon libraries generated using the methods described herein are in some instances subjected to additional steps, such as adapter ligation and further PCR amplification. In some instances, such additional steps precede a sequencing step.

Primers comprise nucleic acids used for priming the amplification reactions described herein. Such primers in some instances include, without limitation, random deoxynucleotides of any length with or without modifications to make them exonuclease resistant, random ribonucleotides of any length with or without modifications to make them exonuclease resistant, modified nucleic acids such as locked nucleic acids, DNA or RNA primers that are targeted to a specific genomic region, and reactions that are primed with enzymes such as primase. In the case of whole genome PTA, it is preferred that a set of primers having random or partially random nucleotide sequences be used. In a nucleic acid sample of significant complexity, specific nucleic acid sequences present in the sample need not be known and the primers need not be designed to be complementary to any particular sequence. Rather, the complexity of the nucleic acid sample results in a large number of different hybridization target sequences in the sample, which will be complementary to various primers of random or partially random sequence. The complementary portion of primers for use in PTA are in some instances fully randomized, comprise only a portion that is randomized, or be otherwise selectively randomized. The number of random base positions in the complementary portion of primers in some instances, for example, is from 20% to 100% of the total number of nucleotides in the complementary portion of the primers. In some instances, the number of random base positions in the complementary portion of primers is 10% to 90%, 15-95%, 20%-100%, 30%-100%, 50%-100%, 75-100% or 90-95% of the total number of nucleotides in the complementary portion of the primers. In some instances, the number of random base positions in the complementary portion of primers is at least 10%, 20%, 30%, 40%, 50%, 60%, 70%, 80%, or at least 90% of the total number of nucleotides in the complementary portion of the primers. Sets of primers having random or partially random sequences are in some instances synthesized using standard techniques by allowing the addition of any nucleotide at each position to be randomized. In some instances, sets of primers are composed of primers of similar length and/or hybridization characteristics. In some instances, the term “random primer” refers to a primer which can exhibit four-fold degeneracy at each position. In some instances, the term “random primer” refers to a primer which can exhibit three-fold degeneracy at each position. Random primers used in the methods described herein in some instances comprise a random sequence that is 3, 4, 5, 6, 7, 8, 10, 11, 12, 13, 14, 15, 16, 17, 18, 19, 20, or more bases in length. In some instances, primers comprise random sequences that are 3-20, 5-15, 5-20, 6-12, or 4-10 bases in length. Primers may also comprise non-extendable elements that limit subsequent amplification of amplicons generated thereof. For example, primers with non-extendable elements in some instances comprise terminators. In some instances, primers comprise terminator nucleotides, such as 1, 2, 3, 4, 5, 10, or more than 10 terminator nucleotides. Primers need not be limited to components which are added externally to an amplification reaction. In some instances, primers are generated in-situ through the addition of nucleotides and proteins which promote priming. For example, primase-like enzymes in combination with nucleotides is in some instances used to generate random primers for the methods described herein. Primase-like enzymes in some instances are members of the DnaG or AEP enzyme superfamily. In some instances, a primase-like enzyme is TthPrimPol. In some instances, a primase-like enzyme is T7 gp4 helicase-primase. Such primases are in some instances used with the polymerases or strand displacement factors described herein. In some instances, primases initiate priming with deoxyribonucleotides. In some instances, primases initiate priming with ribonucleotides. In some instances, primers are irreversible primers. In some instances, irreversible primers comprise phosphonothioate linkages.

The PTA amplification can be followed by selection for a specific subset of amplicons. Such selections are in some instances dependent on size, affinity, activity, hybridization to probes, or other known selection factor in the art. In some instances, selections precede or follow additional steps described herein, such as adapter ligation and/or library amplification. In some instances, selections are based on size (length) of the amplicons. In some instances, smaller amplicons are selected that are less likely to have undergone exponential amplification, which enriches for products that were derived from the primary template while further converting the amplification from an exponential into a quasi-linear amplification process. In some instances, amplicons comprising 50-2000, 25-5000, 40-3000, 50-1000, 200-1000, 300-1000, 400-1000, 400-600, 600-2000, or 800-1000 bases in length are selected. Size selection in some instances occurs with the use of protocols, e.g., utilizing solid-phase reversible immobilization (SPRI) on carboxylated paramagnetic beads to enrich for nucleic acid fragments of specific sizes, or other protocol known by those skilled in the art. Optionally or in combination, selection occurs through preferential ligation and amplification of smaller fragments during PCR while preparing sequencing libraries, as well as a result of the preferential formation of clusters from smaller sequencing library fragments during sequencing (e.g., sequencing by synthesis, nanopore sequencing, or other sequencing method). Other strategies to select for smaller fragments are also consistent with the methods described herein and include, without limitation, isolating nucleic acid fragments of specific sizes after gel electrophoresis, the use of silica columns that bind nucleic acid fragments of specific sizes, and the use of other PCR strategies that more strongly enrich for smaller fragments. Any number of library preparation protocols may be used with the PTA methods described herein. Amplicons generated by PTA are in some instances ligated to adapters (optionally with removal of terminator nucleotides). In some instances, amplicons generated by PTA comprise regions of homology generated from transposase-based fragmentation which are used as priming sites. In some instances, libraries are prepared by fragmenting nucleic acids mechanically or enzymatically. In some instances, libraries are prepared using tagmentation via transposomes. In some instances, libraries are prepared via ligation of adapters, such as Y-adapters, universal adapters, or circular adapters.

The non-complementary portion of a primer used in PTA can include sequences which can be used to further manipulate and/or analyze amplified sequences. An example of such a sequence is a “detection tag”. Detection tags have sequences complementary to detection probes and are detected using their cognate detection probes. There may be one, two, three, four, or more than four detection tags on a primer. There is no fundamental limit to the number of detection tags that can be present on a primer except the size of the primer. In some instances, there is a single detection tag on a primer. In some instances, there are two detection tags on a primer. When there are multiple detection tags, they may have the same sequence or they may have different sequences, with each different sequence complementary to a different detection probe. In some instances, multiple detection tags have the same sequence. In some instances, multiple detection tags have a different sequence.

Another example of a sequence that can be included in the non-complementary portion of a primer is an “address tag” that can encode other details of the amplicons, such as the location in a tissue section. In some instances, a cell barcode comprises an address tag. An address tag has a sequence complementary to an address probe. Address tags become incorporated at the ends of amplified strands. If present, there may be one, or more than one, address tag on a primer. There is no fundamental limit to the number of address tags that can be present on a primer except the size of the primer. When there are multiple address tags, they may have the same sequence or they may have different sequences, with each different sequence complementary to a different address probe. The address tag portion can be any length that supports specific and stable hybridization between the address tag and the address probe. In some instances, nucleic acids from more than one source can incorporate a variable tag sequence. This tag sequence can be up to 100 nucleotides in length, preferably 1 to 10 nucleotides in length, most preferably 4, 5 or 6 nucleotides in length and comprises combinations of nucleotides. In some instances, a tag sequence is 1-20, 2-15, 3-13, 4-12, 5-12, or 1-10 nucleotides in length For example, if six base-pairs are chosen to form the tag and a permutation of four different nucleotides is used, then a total of 4096 nucleic acid anchors (e.g. hairpins), each with a unique 6 base tag can be made. In some instances, tags identify the source of a sample or analyte. In some instances, tags uniquely identify every molecule in a population.

Primers described herein may be present in solution or immobilized on a solid support. In some instances, primers bearing sample barcodes and/or UMI sequences can be immobilized on a solid support. The solid support can be, for example, one or more beads. In some instances, individual cells are contacted with one or more beads having a unique set of sample barcodes and/or UMI sequences in order to identify the individual cell. In some instances, lysates from individual cells are contacted with one or more beads having a unique set of sample barcodes and/or UMI sequences in order to identify the individual cell lysates. In some instances, extracted nucleic acid from individual cells are contacted with one or more beads having a unique set of sample barcodes and/or UMI sequences in order to identify the extracted nucleic acid from the individual cell. The beads can be manipulated in any suitable manner as is known in the art, for example, using droplet actuators as described herein. The beads may be any suitable size, including for example, microbeads, microparticles, nanobeads and nanoparticles. In some embodiments, beads are magnetically responsive; in other embodiments beads are not significantly magnetically responsive. Non-limiting examples of suitable beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® available from Invitrogen Group, Carlsbad, CA), fluorescent microparticles and nanoparticles, coated magnetic microparticles and nanoparticles, ferromagnetic microparticles and nanoparticles, coated ferromagnetic microparticles and nanoparticles, and those described in U.S. Pat. Appl. Pub. No. US20050260686, US20030132538, US20050118574, 20050277197, 20060159962. Beads may be pre-coupled with an antibody, protein or antigen, DNA/RNA probe or any other molecule with an affinity for a desired target. In some embodiments, primers bearing sample barcodes and/or UMI sequences can be in solution. In certain embodiments, a plurality of droplets can be presented, wherein each droplet in the plurality bears a sample barcode which is unique to a droplet and the UMI which is unique to a molecule such that the UMI are repeated many times within a collection of droplets. In some embodiments, individual cells are contacted with a droplet having a unique set of sample barcodes and/or UMI sequences in order to identify the individual cell. In some embodiments, lysates from individual cells are contacted with a droplet having a unique set of sample barcodes and/or UMI sequences in order to identify the individual cell lysates. In some embodiments, extracted nucleic acid from individual cells are contacted with a droplet having a unique set of sample barcodes and/or UMI sequences in order to identify the extracted nucleic acid from the individual cell.

PTA primers may comprise a sequence-specific or random primer, a cell barcode and/or a unique molecular identifier (UMI) (e.g., linear primer and or hairpin primer). In some instances, a primer comprises a sequence-specific primer. In some instances, a primer comprises a random primer. In some instances, a primer comprises a cell barcode. In some instances, a primer comprises a sample barcode. In some instances, a primer comprises a unique molecular identifier. In some instances, primers comprise two or more cell barcodes. Such barcodes in some instances identify a unique sample source, or unique workflow. Such barcodes or UMIs are in some instances 5, 6, 7, 8, 9, 10, 11, 12, 15, 20, 25, 30, or more than 30 bases in length. Primers in some instances comprise at least 1000, 10,000, 50,000, 100,000, 250,000, 500,000, 106, 107, 108, 109, or at least 1010 unique barcodes or UMIs. In some instances primers comprise at least 8, 16, 96, 384, or 1536 or more unique barcodes or UMIs. In some instances a standard adapter is then ligated onto the amplification products prior to sequencing; after sequencing, reads are first assigned to a specific cell based on the cell barcode. Suitable adapters that may be utilized with the PTA method include, e.g., xGen® Dual Index UMI adapters available from Integrated DNA Technologies (IDT). Reads from each cell is then grouped using the UMI, and reads with the same UMI may be collapsed into a consensus read. The use of a cell barcode allows all cells to be pooled prior to library preparation, as they can later be identified by the cell barcode. The use of the UMI to form a consensus read in some instances corrects for PCR bias, improving the copy number variation (CNV) detection. In addition, sequencing errors may be corrected by requiring that a fixed percentage of reads from the same molecule have the same base change detected at each position. This approach has been utilized to improve CNV detection and correct sequencing errors in bulk samples. In some instances, UMIs are used with the methods described herein, for example, U.S. Pat. No. 8,835,358 discloses the principle of digital counting after attaching a random amplifiable barcode. Schmitt. et al and Fan et al. disclose similar methods of correcting sequencing errors. In some instances, a library is generated for sequencing using primers. In some instances, the library comprises fragments of 200-700 bases, 100-1000, 300-800, 300-550, 300-700, or 200-800 bases in length. In some instances, the library comprises fragments of at least 50, 100, 150, 200, 300, 500, 600, 700, 800, or at least 1000 bases in length. In some instances, the library comprises fragments of about 50, 100, 150, 200, 300, 500, 600, 700, 800, or about 1000 bases in length.

The methods described herein may further comprise additional steps, including steps performed on the sample or template. Such samples or templates in some instance are subjected to one or more steps prior to PTA. In some instances, samples comprising cells are subjected to a pre-treatment step. For example, cells undergo lysis and proteolysis to increase chromatin accessibility using a combination of freeze-thawing, Triton X-1-- (e.g., 100, 114, etc.), Tween 20, and Proteinase K. Other lysis strategies are also suitable for practicing the methods described herein. Such strategies include, without limitation, lysis using other combinations of detergent and/or lysozyme and/or protease treatment and/or physical disruption of cells such as sonication and/or alkaline lysis and/or hypotonic lysis. In some instances, the primary template or target molecule(s) is subjected to a pre-treatment step. In some instances, the primary template (or target) is denatured using sodium hydroxide, followed by neutralization of the solution. Other denaturing strategies may also be suitable for practicing the methods described herein. Such strategies may include, without limitation, combinations of alkaline lysis with other basic solutions, increasing the temperature of the sample and/or altering the salt concentration in the sample, addition of additives such as solvents or oils, other modification, or any combination thereof. In some instances, additional steps include sorting, filtering, or isolating samples, templates, or amplicons by size. In some instances, cells are lysed with mechanical (e.g., high pressure homogenizer, bead milling) or non-mechanical (physical, chemical, or biological). In some instances, physical lysis methods comprise heating, osmotic shock, and/or cavitation. In some instances, chemical lysis comprises alkali and/or detergents. In some instances, biological lysis comprises use of enzymes. Combinations of lysis methods are also compatible with the methods described herein. Non-limited examples of lysis enzymes include recombinant lysozyme, serine proteases, and bacterial lysins. In some instances, lysis with enzymes comprises use of lysozyme, lysostaphin, zymolase, cellulose, protease or glycanase. For example, after amplification with the methods described herein, amplicon libraries are enriched for amplicons having a desired length. In some instances, amplicon libraries are enriched for amplicons having a length of 50-2000, 25-1000, 50-1000, 75-2000, 100-3000, 150-500, 75-250, 170-500, 100-500, or 75-2000 bases. In some instances, amplicon libraries are enriched for amplicons having a length no more than 75, 100, 150, 200, 500, 750, 1000, 2000, 5000, or no more than 10,000 bases. In some instances, amplicon libraries are enriched for amplicons having a length of at least 25, 50, 75, 100, 150, 200, 500, 750, 1000, or at least 2000 bases.

Methods and compositions described herein may comprise buffers or other formulations. Such buffers are in some instances used for PTA, RT, or other method described herein. Such buffers in some instances comprise surfactants/detergent or denaturing agents (Tween-20, DMSO, DMF, pegylated polymers comprising a hydrophobic group, or other surfactant), salts (potassium or sodium phosphate (monobasic or dibasic), sodium chloride, potassium chloride, TrisHCI, magnesium chloride or sulfate, Ammonium salts such as phosphate, nitrate, or sulfate, EDTA), reducing agents (DTT, THP, DTE, beta-mercaptoethanol, TCEP, or other reducing agent) or other components (glycerol, hydrophilic polymers such as PEG). In some instances, buffers are used in conjunction with components such as polymerases, strand displacement factors, terminators, or other reaction component described herein. In some instances, buffers are used in conjunction with components such as polymerases, strand displacement factors, terminators, or other reaction component described herein. Buffers may comprise one or more crowding agents. In some instances, crowding reagents include polymers. In some instances, crowding reagents comprise polymers such as polyols. In some instances, crowding reagents comprise polyethylene glycol polymers (PEG). In some instances, crowding reagents comprise polysaccharides. Without limitation, examples of crowding reagents include ficoll (e.g., ficoll PM 400, ficoll PM 70, or other molecular weight ficoll), PEG (e.g., PEG1000, PEG 2000, PEG4000, PEG6000, PEG8000, or other molecular weight PEG), dextran (dextran 6, dextran 10, dextran 40, dextran 70, dextran 6000, dextran 138k, or other molecular weight dextran).

The nucleic acid molecules amplified according to the methods described herein may be sequenced and analyzed using methods known to those of skill in the art. Non-limiting examples of the sequencing methods which in some instances are used include, e.g., sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (Int. Pat. Appl. Pub. No. WO2006/073504), multiplex sequencing (U.S. Pat. Appl. Pub. No. US2008/0269068; Porreca et al., 2007, Nat. Methods 4:931), polymerized colony (POLONY) sequencing (U.S. Pat. Nos. 6,432,360, 6,485,944 and 6,511,803, and Int. Pat. Appl. Pub. No. WO2005/082098), nanogrid rolling circle sequencing (ROLONY) (U.S. Pat. No. 9,624,538), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout), high-throughput sequencing methods such as, e.g., methods using Roche 454, Illumina Solexa, AB-SOLID, Helicos, Polonator platforms and the like, and light-based sequencing technologies (Landegren et al. (1998) Genome Res. 8:769-76; Kwok (2000) Pharmacogenomics 1:95-100; and Shi (2001) Clin. Chem.47:164-172). In some instances, the amplified nucleic acid molecules are shotgun sequenced. Sequencing of the sequencing library is in some instances performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis (array/colony-based or nanoball based).

Sequencing libraries generated using the methods described herein (e.g., PTA or RNAseq) may be sequenced to obtain a desired number of sequencing reads. In some instances, libraries are generated from a single cell or sample comprising a single cell (alone or part of a multiomics workflow). In some instances, libraries are sequenced to obtain at least 0.1, 0.2, 0.4, 0.5, 0.7, 0.8, 0.9, 1, 1.1, 1.2, 1.5, 2, 5, or at least 10 million reads. In some instances, libraries are sequenced to obtain no more than 0.1, 0.2, 0.4, 0.5, 0.7, 0.8, 0.9, 1, 1.1, 1.2, 1.5, 2, 5, or no more than 10 million reads. In some instances, libraries are sequenced to obtain about 0.1, 0.2, 0.4, 0.5, 0.7, 0.8, 0.9, 1, 1.1, 1.2, 1.5, 2, 5, or about 10 million reads. In some instances, libraries are sequenced to obtain 0.1-10, 0.1-5, 0.1-1, 0.2-1, 0.3-1.5, 0.5-1, 1-5, or 0.5-5 million reads per sample. In some instances, the number of reads is dependent on the size of the genome. In some in instances samples comprising bacterial genomes are sequenced to obtain 0.5-1 million reads. In some instances, libraries are sequenced to obtain at least 2, 4, 10, 20, 50, 100, 200, 300, 500, 700, or at least 900 million reads. In some instances, libraries are sequenced to obtain no more than 2, 4, 10, 20, 50, 100, 200, 300, 500, 700, or no more than 900 million reads. In some instances, libraries are sequenced to obtain about 2, 4, 10, 20, 50, 100, 200, 300, 500, 700, or about 900 million reads. In some in instances samples comprising mammalian genomes are sequenced to obtain 500-600 million reads. In some instances, the type of sequencing library (cDNA libraries or genomic libraries) are identified during sequencing. In some instances, cDNA libraries and genomic libraries are identified during sequencing with unique barcodes.

The term “cycle” when used in reference to a polymerase-mediated amplification reaction is used herein to describe steps of dissociation of at least a portion of a double stranded nucleic acid (e.g., a template from an amplicon, or a double stranded template, denaturation). hybridization of at least a portion of a primer to a template (annealing), and extension of the primer to generate an amplicon. In some instances, the temperature remains constant during a cycle of amplification (e.g., an isothermal reaction). In some instances, the number of cycles is directly correlated with the number of amplicons produced. In some instances, the number of cycles for an isothermal reaction is controlled by the amount of time the reaction is allowed to proceed.

### High Throughput Methods and Applications

High throughput devices and methods described herein may be used for a number of applications. Described herein are methods of identifying mutations in cells with the methods of multiomic analysis PTA, such as single cells. Use of the PTA method in some instances results in improvements over known methods, for example, MDA. PTA in some instances has lower false positive and false negative variant calling rates than the MDA method. Genomes, such as NA12878 platinum genomes, are in some instances used to determine if the greater genome coverage and uniformity of PTA would result in lower false negative variant calling rate. Without being bound by theory, it may be determined that the lack of error propagation in PTA decreases the false positive variant call rate. The amplification balance between alleles with the two methods is in some cases estimated by comparing the allele frequencies of the heterozygous mutation calls at known positive loci. In some instances, amplicon libraries generated using PTA are further amplified by PCR. In some instances, PTA is used in a workflow with additional analysis methods, such as RNAseq, methylome analysis or other method described herein.

Cells analyzed using the methods described herein in some instances comprise tumor cells. For example, circulating tumor cells can be isolated from a fluid taken from patients, such as but not limited to, blood, bone marrow, urine, saliva, cerebrospinal fluid, pleural fluid, pericardial fluid, ascites, or aqueous humor. The cells are then subjected to the methods described herein (e.g. PTA) and sequencing to determine mutation burden and mutation combination in each cell. These data are in some instances used for the diagnosis of a specific disease or as tools to predict treatment response. Similarly, in some instances cells of unknown malignant potential in some instances are isolated from fluid taken from patients, such as but not limited to, blood, bone marrow, urine, saliva, cerebrospinal fluid, pleural fluid, pericardial fluid, ascites, aqueous humor, blastocoel fluid, or collection media surrounding cells in culture. In some instances, a sample is obtained from collection media surrounding embryonic cells. . . . After utilizing the methods described herein and sequencing, such methods are further used to determine mutation burden and mutation combination in each cell. These data are in some instances used for the diagnosis of a specific disease or as tools to predict progression of a premalignant state to overt malignancy. In some instances, cells can be isolated from primary tumor samples. The cells can then undergo PTA and sequencing to determine mutation burden and mutation combination in each cell. These data can be used for the diagnosis of a specific disease or are as tools to predict the probability that a patient's malignancy is resistant to available anti-cancer drugs. By exposing samples to different chemotherapy agents, it has been found that the major and minor clones have differential sensitivity to specific drugs that does not necessarily correlate with the presence of a known “driver mutation,” suggesting that combinations of mutations within a clonal population determine its sensitivities to specific chemotherapy drugs. Without being bound by theory, these findings suggest that a malignancy may be easier to eradicate if premalignant lesions that have not yet expanded are and evolved into clones are detected whose increased number of genome modification may make them more likely to be resistant to treatment. See, Ma et al., 2018, “Pan-cancer genome and transcriptome analyses of 1,699 pediatric leukemias and solid tumors.” A single-cell genomics protocol is in some instances used to detect the combinations of somatic genetic variants in a single cancer cell, or clonotype, within a mixture of normal and malignant cells that are isolated from patient samples. This technology is in some instances further utilized to identify clonotypes that undergo positive selection after exposure to drugs, both in vitro and/or in patients. By comparing the surviving clones exposed to chemotherapy compared to the clones identified at diagnosis, a catalog of cancer clonotypes can be created that documents their resistance to specific drugs. PTA methods in some instances detect the sensitivity of specific clones in a sample composed of multiple clonotypes to existing or novel drugs, as well as combinations thereof, where the method can detect the sensitivity of specific clones to the drug. This approach in some instances shows efficacy of a drug for a specific clone that may not be detected with current drug sensitivity measurements that consider the sensitivity of all cancer clones together in one measurement. When the PTA described herein are applied to patient samples collected at the time of diagnosis in order to detect the cancer clonotypes in a given patient's cancer, a catalog of drug sensitivities may then be used to look up those clones and thereby inform oncologists as to which drug or combination of drugs will not work and which drug or combination of drugs is most likely to be efficacious against that patient's cancer. The PTA may be used for analysis of samples comprising groups of cells. In some instances, a sample comprises neurons or glial cells. In some instances, the sample comprises nuclei.

Described herein are methods of measuring the gene expression alteration in combination with the mutagenicity of an environmental factor. For example, cells (single or a population) are exposed to a potential environmental condition. For example, cells such originating from organs (liver, pancreas, lung, colon, thyroid, or other organ), tissues (skin, or other tissue), blood, or other biological source are in some instances used with the method. In some instances, an environmental condition comprises heat, light (e.g. ultraviolet), radiation, a chemical substance, or any combination thereof. After an amount of exposure to the environmental condition, in some instances minutes, hours, days, or longer, single cells are isolated and subjected to the PTA method. In some instances, molecular barcodes and unique molecular identifiers are used to tag the sample. The sample is sequenced and then analyzed to identify gene expression alterations and or resulting from mutations resulting from exposure to the environmental condition. In some instances, such mutations are compared with a control environmental condition, such as a known non-mutagenic substance, vehicle/solvent, or lack of an environmental condition. Such analysis in some instances not only provides the total number of mutations caused by the environmental condition, but also the locations and nature of such mutations. Patterns are in some instances identified from the data, and may be used for diagnosis of diseases or conditions. In some instances, patterns are used to predict future disease states or conditions. In some instances, the methods described herein measure the mutation burden, locations, and patterns in a cell after exposure to an environmental agent, such as, e.g., a potential mutagen or teratogen. This approach in some instances is used to evaluate the safety of a given agent, including its potential to induce mutations that can contribute to the development of a disease. For example, the method could be used to predict the carcinogenicity or teratogenicity of an agent to specific cell types after exposure to a specific concentration of the specific agent.

Described herein are methods of identifying gene expression alteration in combination with the mutations in animal, plant or microbial cells that have undergone genome editing (e.g., using CRISPR technologies). Such cells in some instances can be isolated and subjected to PTA and sequencing to determine mutation burden and mutation combination in each cell. The per-cell mutation rate and locations of mutations that result from a genome editing protocol are in some instances used to assess the safety of a given genome editing method.

Described herein are methods of determining gene expression alteration in combination with the mutations in cells that are used for cellular therapy, such as but not limited to the transplantation of induced pluripotent stem cells, transplantation of hematopoietic or other cells that have not be manipulated, or transplantation of hematopoietic or other cells that have undergone genome edits. The cells can then undergo PTA and sequencing to determine mutation burden and mutation combination in each cell. The per-cell mutation rate and locations of mutations in the cellular therapy product can be used to assess the safety and potential efficacy of the product.

Cells for use with the PTA method may be fetal cells, such as embryonic cells. In some embodiments, PTA is used in conjunction with non-invasive preimplantation genetic testing (NIPGT). In a further embodiment, cells can be isolated from blastomeres that are created by in vitro fertilization. The cells can then undergo PTA and sequencing to determine the burden and combination of potentially disease predisposing genetic variants in each cell. The gene expression alteration in combination with the mutation profile of the cell can then be used to extrapolate the genetic predisposition of the blastomere to specific diseases prior to implantation. In some instances embryos in culture shed nucleic acids that are used to assess the health of the embryo using low pass genome sequencing. In some instances, embryos are frozen-thawed. In some instances, nucleic acids are obtained from blastocyte culture conditioned medium (BCCM), blastocoel fluid (BF), or a combination thereof. In some instances, PTA analysis of fetal cells is used to detect chromosomal abnormalities, such as fetal aneuploidy. In some instances, PTA is used to detect diseases such as Down's or Patau syndromes. In some instances, frozen blastocytes are thawed and cultured for a period of time before obtaining nucleic acids for analysis (e.g., culture media, BF, or a cell biopsy). In some instances, blastocytes are cultured for no more than 4, 6, 8, 12, 16, 24, 36, 48, or no more than 64 hours prior to obtaining nucleic acids for analysis.

In another embodiment, microbial cells (e.g., bacteria, fungi, protozoa) can be isolated from plants or animals (e.g., from microbiota samples [e.g., GI microbiota, skin microbiota, etc.] or from bodily fluids such as, e.g., blood, bone marrow, urine, saliva, cerebrospinal fluid, pleural fluid, pericardial fluid, ascites, or aqueous humor). In addition, microbial cells may be isolated from indwelling medical devices, such as but not limited to, intravenous catheters, urethral catheters, cerebrospinal shunts, prosthetic valves, artificial joints, or endotracheal tubes. The cells can then undergo PTA and sequencing to determine the identity of a specific microbe, as well as to detect the presence of microbial genetic variants that predict response (or resistance) to specific antimicrobial agents. These data can be used for the diagnosis of a specific infectious disease and/or as tools to predict treatment response.

Described herein are methods generating amplicon libraries from samples comprising short nucleic acid using the PTA methods described herein. In some instances, PTA leads to improved fidelity and uniformity of amplification of shorter nucleic acids. In some instances, nucleic acids are no more than 2000 bases in length. In some instances, nucleic acids are no more than 1000 bases in length. In some instances, nucleic acids are no more than 500 bases in length. In some instances, nucleic acids are no more than 200, 400, 750, 1000, 2000 or 5000 bases in length. In some instances, samples comprising short nucleic acid fragments include but at not limited to ancient DNA (hundreds, thousands, millions, or even billions of years old), FFPE (Formalin-Fixed Paraffin-Embedded) samples, cell-free DNA, or other sample comprising short nucleic acids.

### EXAMPLES

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### Example 1: Primary Template-Directed Amplification

Single Cell Capture by FACS Sorting. A low bind 96-well PCR plate was placed on a PCR cooler. 3 μL of Cell Buffer was added to all the wells where cells will be sorted. The plate was sealed with a sealing film and kept it on ice until ready to use. After single cell sorting, the plate is sealed. The plate was mixed for 10 seconds at 1400 RPM on a PCR Plate Thermal Mixer at room temperature, spun briefly, and placed on ice. Alternatively, plates containing sorted cells were stored on dry ice with a seal or at −80° C. until ready.

Single Cell Whole Genome Amplification with PTA. After adding reagents to plates containing cells, an RPM controlled mixer was used PCR cooler at set to −20° C. for 2 hrs and thawed for 10 min or alternatively the following reactions were conducted on ice. Reactions were assembled in a DNA-free pre-PCR hood. All reagents were thawed on ice until ready to use. Before use, each reagent was vortexed for 10 sec and spun briefly. Reagents were dispensed to the wall of the tube without touching cell suspension. 96-well PCR plate containing cells were placed on the PCR cooler. If cells were stored at −80° C., cells were thawed on ice for 5 minutes, spun for 10 seconds, then the plate placed on the PCR cooler (or ice). 1× Reagent Mix was prepared by diluting 12× mix, mixing on the vortexer, and briefly spinning tube. MS Mix was prepared by combining 1× reagent mix and lysis buffer, mixing on the vortexer, and briefly spinning tube. 3 μL of MS Mix was added to each well of the plate, and the plate was sealed with the sealing film. After spinning for 10 sec, mixing at room temperature for 1 min at 1400 rpm (plate mixer), and spinning for 10 sec, plate was placed back on PCR cooler (or ice) for 10 minutes. 3 μL of neutralization buffer, was then added, and the plate was sealed with the plate film. After spinning for 10 sec, mixing at room temperature for 1 min at 1400 rpm (plate mixer), spinning for 10 sec, the plate was placed back on the PCR cooler. 3 μL of buffer was added, and the plate sealed with the plate film. Next, the plate was spun for 10 sec, mixed at room temperature for 1 min at 1400 rpm (plate mixer), and spun for 10 sec followed by incubating at room temperature for 10 min. During the incubation step, the Reaction Mix was prepared by combining the components in the order (nucleotide/terminator reagents, 5.0 μL; 1× reagent mix, 1.0 μL; Phi29 polymerase, 0.8 μL; singe-stranded binding protein reagent, 1.2 μL), followed by mixing gently and thoroughly by pipetting up and down 10 times, then spun briefly. When the incubation is completed, the plate is placed on the PCR cooler (or ice). 8 μL of Reaction Mix was added to each sample while the plate is still on the PCR cooler (or ice), and mixed at room temperature for 1 min at 1000 rpm in plate mixer, then spun briefly. The plate is placed on a thermal cycler (lid set to 70° C.) with the following program: 30° C. for 10 hrs, 65° C. for 3 min, 4° C. hold.

Amplified DNA Cleanup. Capture beads were allowed to equilibrate to room temperature for 30 min. Beads are mixed thoroughly, and then 40 μL of beads were added to each reaction well (vortex and spin). Beads were aspirated prior to each dispensing step, incubated at room temperature for 10 minutes, and the sample plate briefly centrifuged. The plate was placed on a magnet for 3 minutes or until the supernatant cleared. While on the magnet, the supernatant is removed and discarded, being careful not to disturb the beads containing DNA. While on magnet, 200 μL of freshly prepared 80% ethanol was added to the beads and incubated for 30 seconds at room temperature. While still on the magnet, the first ethanol wash is removed and discarded, taking care not to disturb the beads. Another 200 μL of freshly prepared 80% ethanol is added to the beads, and then incubated for 30 seconds at room temperature. The second ethanol wash is then removed and discarded, taking care not to disturb the beads. Any remaining ethanol from the wells is discarded. The beads are then incubated at room temperature for 5 minutes to air-dry beads, then the plate was removed from the magnet. Beads were then re-suspended in 40 μL of elution buffer, incubated for 2 minutes at room temperature, and placed on the magnet for 3 minutes, or until the supernatant clears. 38 μL of the eluted DNA was transferred to a new plate, for DNA quantification. DNA was then ready to use in downstream applications such as PCR or Real Time PCR.

DNA Quantification. Quantitate DNA using the High Sensitivity dsDNA Assay kit (Qubit) as per manufacturer. Size fragment analysis was completed to ensure proper amplification product size. Fragment size distribution was determined by running 1 μL of PTA product on an E-Gel EX, or 1 μL of 2 ng/μL in a High Sensitivity Bioanalyzer DNA Chip.

End Repair and A-tailing. 500 ng of amplified DNA was added to a PCR tube. DNA volume was adjusted to 35 μL with RT-PCR grade water. The End-Repair A-Tail Reaction was assembled on a PCR cooler (or ice) as follows: Amplified DNA (500 ng total DNA/Rxn, 35 μL), RT-PCR grade water (10 μL), fragmentation buffer (5 μL), ER/AT buffer (7 μL), ER/AT enzyme (3 μL) to a total volume of 60 μL, which was mixed thoroughly and spun briefly. The mixture was then incubated at 65° C. on a thermal cycler with the lid at 105° C. for 30 minutes.

Adapter Ligation. Multi-Use Library Adapters stock plate was diluted to 1× by adding 54 μL of 10 mM Tris-HCl, 0.1 mM EDTA, pH 8.0 to each well. In the same plate/tube(s) in which end-repair and A-tailing was performed, each Adapter Ligation Reaction was assembled as follows: ER/AT DNA (60 μL), 1× Multi-Use Library Adapters (5 μL), RT-PCR grade water (5 μL), ligation buffer (30 μL), and DNA ligase (10 μL) to a total volume of 110 μL. After thorough mixing and brief spin, the mixture is incubated at 20° C. on thermal cycler for 15 minutes (heated lid not required).

Post Ligation Cleanup. Beads were allowed to equilibrate to room temperature for 30 minutes then mixed thoroughly and immediately before pipetting. In the same plate/tube(s), a 0.8×SPRI cleanup was assembled as follows: adapter-ligated DNA (110 μL), and beads (88 μL) to a final volume of 198 μL. The mixture is mixed thoroughly and incubated for 10 min at room temperature, and the plate/tube(s) are placed on the magnet for 2 minutes, or until the supernatant clears. While on the magnet, the supernatant was removed and discarded being careful not to disturb any beads, followed by washing with 200 μL of freshly prepared 80% ethanol to the beads and incubating for 30 seconds at room temperature. While still on the magnet, the first ethanol wash is removed and discarded, taking care not to disturb the beads. Another 200 μL of freshly prepared 80% ethanol is added to the beads, and then incubated for 30 seconds at room temperature. The second ethanol wash is then removed and discarded, taking care not to disturb the beads. Any remaining ethanol from the wells is discarded. The beads are then incubated at room temperature for 5 minutes to air-dry beads, then the plate was removed from the magnet. Beads were then re-suspended in 20 μL of elution buffer, incubated for 2 minutes at room temperature, and placed on the magnet for 3 minutes, or until the supernatant clears.

Library Amplification. In the same plate/tube(s) containing the DNA-Bead slurry, each library amplification reaction is assembled as follows: adapter ligated library (20 μL), 10×KAPA library amplification primer mix (5 μL), and 2×KAPA HiFi Hotstart ready mix (25 μL) to a total volume of 50 μL. After mixing thoroughly and spinning briefly, amplification is conducted using the cycling protocol: Initial Denaturation 98° C. @ 45 sec (1 cycle), Denaturation 98° C. @ 15 sec; Annealing 60° C. 30 sec; and Extension 72° C. 30 sec (10 cycles), Final Extension 72° C. @ 1 min for 1 cycle, and HOLD 4° C. indefinitely. The heated lid was set to 105° C. The plate/tube(s) were stored at 4° C. for up to 72 hours, or directly used for Post-Amplification Cleanup.

Post Amplification Clean up. Beads were allowed to equilibrate to room temperature for 30 minutes. Beads thoroughly and immediately before pipetting, and in the same plate/tube(s), a 0.55×SPRI cleanup was assembled as follows: amplified library (50.0 μL) and beads (27.5 μL) to a total volume of 77.5 μL, followed by thorough mixing and incubation for 10 min at room temperature. Plate/tube(s) were placed on the magnet for 3 minutes, or until the supernatant clears. While on the magnet, the supernatant was transferred to a new plate/tube(s) being careful not to transfer any beads.

In a plate/tube(s), a 0.25×SPRI cleanup was assembled as follows: 0.55× Cleanup Supernatant (77.5 μL), and beads (12.5 μL) to a total volume of 90.0 μL. After thorough mixing, the mixture was spun down and incubated for 10 min at room temperature. Plate/tube(s) were placed on the magnet for 3 minutes or until the supernatant clears. While on the magnet, the supernatant was removed and discarded being careful not to disturb any beads, followed by washing with 200 μL of freshly prepared 80% ethanol to the beads and incubating for 30 seconds at room temperature. While still on the magnet, the first ethanol wash is removed and discarded, taking care not to disturb the beads. Another 200 μL of freshly prepared 80% ethanol is added to the beads, and then incubated for 30 seconds at room temperature. The second ethanol wash is then removed and discarded, taking care not to disturb the beads. Any remaining ethanol from the wells is discarded. The beads are then incubated at room temperature for 5 minutes to air-dry beads, then the plate was removed from the magnet. Beads were then re-suspended in 42 μL of elution buffer, incubated for 2 minutes at room temperature, and placed on the magnet for 3 minutes, or until the supernatant clears. 40 μL of the eluted DNA was transferred to a new plate, for DNA quantification.

Library Quantification. The amplified library is quantitated using a Qubit dsDNA kit as per manufacturer. Fragment size distribution was determined by running 1 μL of library on an E-Gel EX, or 1 μL of 2 ng/μL in a Bioanalyzer DNA Chip.

### Example 2: Methods for High-Throughput Primary Template-Directed Amplification

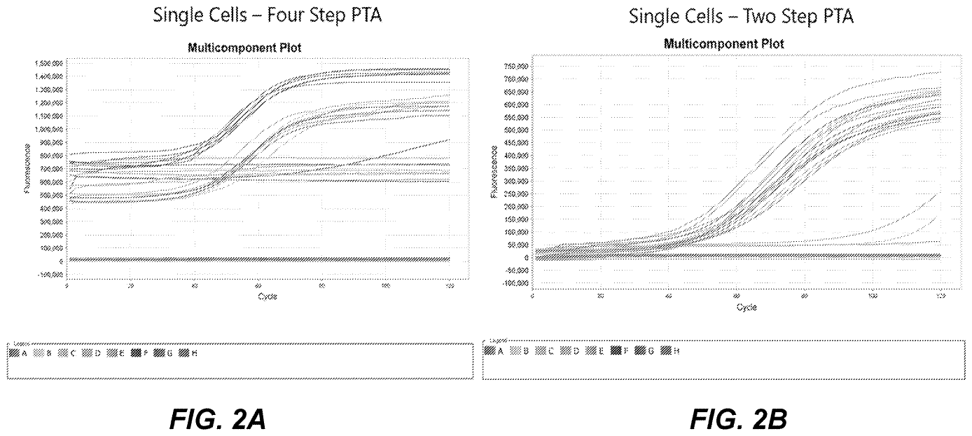

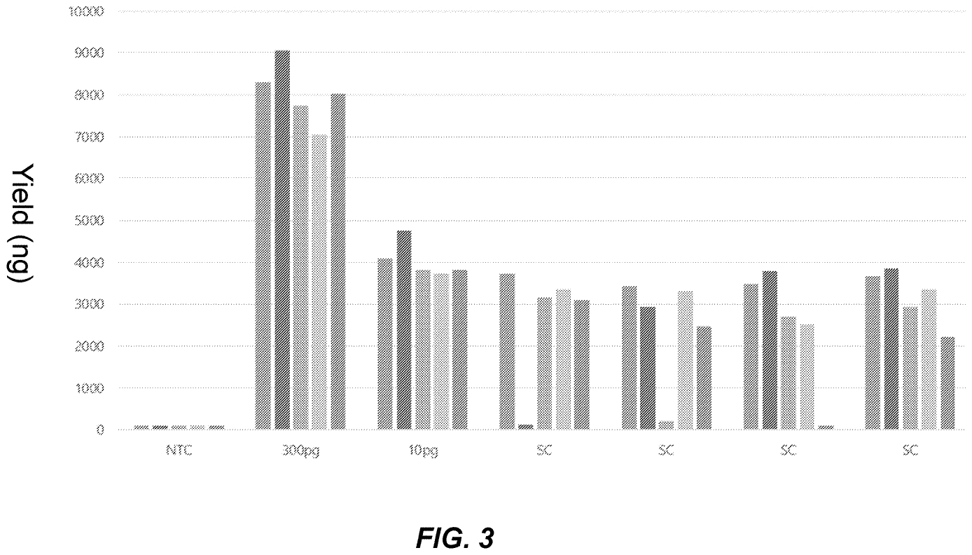

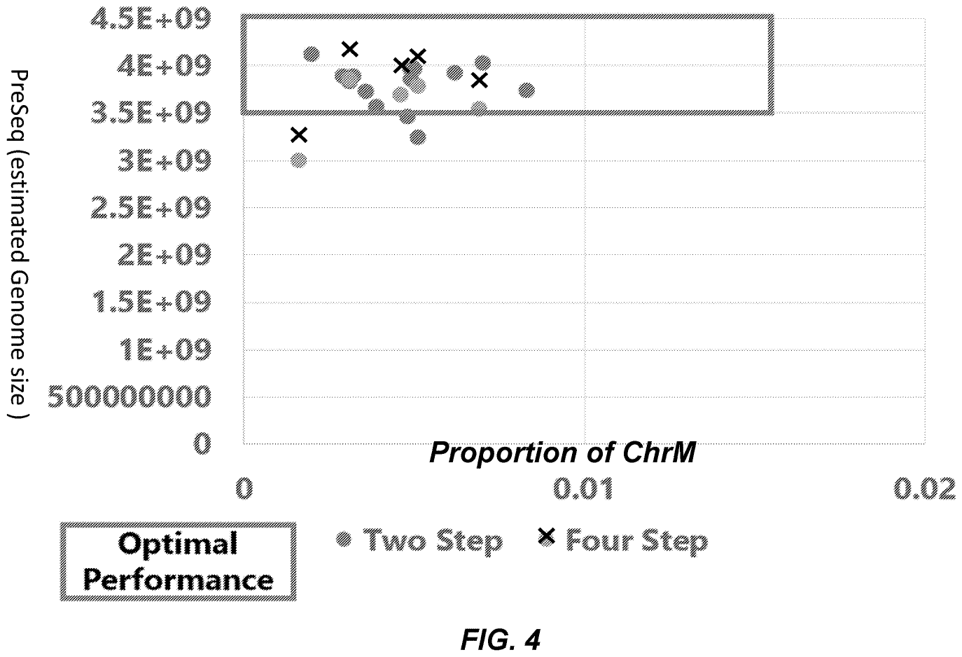

Following the general methods of Example 1, single cells were analyzed with modification: a sub group of experiments was conducted using a two-step PTA protocol involving lysis of cells and priming followed by direct addition of the PTA reaction mixture comprising the neutralization buffer (FIG. 1A-1B). The neutralization buffer comprised HEPES. Results obtained using the two step protocol resulted in comparable yields of amplificated products (FIG. 2A-2B). Different replicates were examined for varying amounts of template or using single cells (FIG. 3). Single cell replicates showing no amplification likely did not receive a cell. Pre-seq values and % mitochondrial reads were also measured, as shown in Table 1 and FIG. 4, demonstrating optimal performance (balance between maximum pre-seq values and minimizing mitochondrial reads) for both two and four step methods.

TABLE 1

| Sample Name | Pre-Seq Value | % Mitochondrial Reads |
|---|---|---|
| H3KVN3-Jay-4Step-SC1 | 2993719098 | 0.0016 |
| H3KVN3-Jay-4Step-SC10 | 3785178288 | 0.0051 |
| H3KVN3-Jay-4Step-SC4 | 3691627375 | 0.0046 |
| H3KVN3-Jay-4Step-SC5 | 3856769543 | 0.0031 |
| H3KVN3-Jay-4Step-SC8 | 3544172893 | 0.0069 |
| H3KVN3-Jeff-2Step-A6 | 3557702262 | 0.0039 |
| H3KVN3-Jeff-2Step-A7 | 3851783864 | 0.0049 |
| H3KVN3-Jeff-2Step-A8 | 3740390108 | 0.0083 |
| H3KVN3-Jeff-2Step-B7 | 3241762990 | 0.0051 |
| H3KVN3-Jeff-2Step-B8 | 3886454651 | 0.0032 |
| H3KVN3-Jeff-2Step-B9 | 3954709876 | 0.0049 |
| H3KVN3-Jeff-2Step-C6 | 3970269923 | 0.005 |

### Example 3: Methods for High-Throughput Primary Template-Directed Amplification

Following the general methods of Examples 1 and 2, single cells were analyzed with modification: concentrations of MgCl, dNTPs, and ddNTPs was varied for both 4-step and 2-step conditions (Table 2). Reactions were run at 2-5 microliters, with most at 3 microliters.

TABLE 2

| Reagent Rxn Conc (mM) | 4-step | [SB4B 2-step] | [SB4W 2-step] |
|---|---|---|---|
| Magnesium Chloride | 8 | 8 | 4.4 |
| dNTPs | 2.8 | 2.8 | 1.25 |
| ddNTPs | 0.3 | 0.3 | 0.15 |

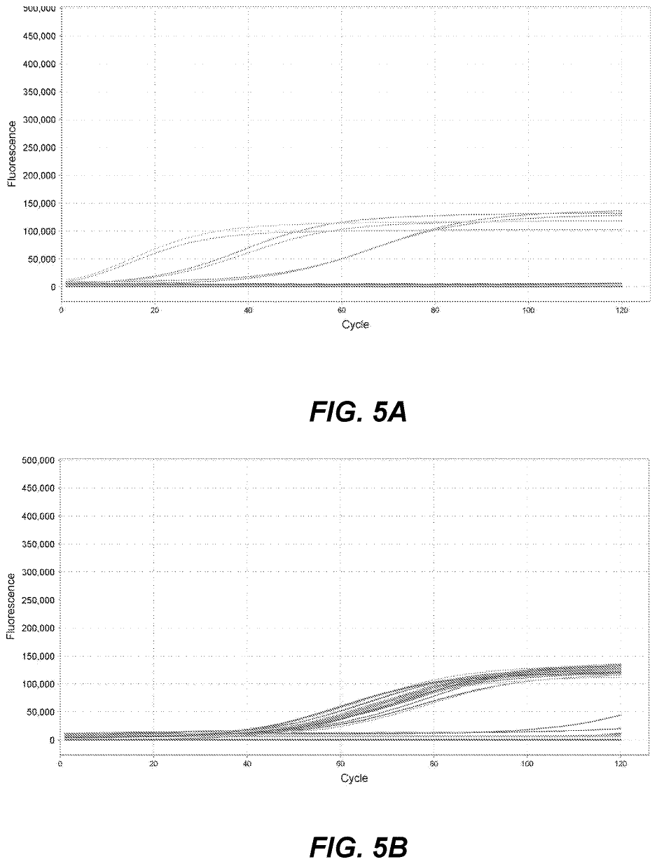

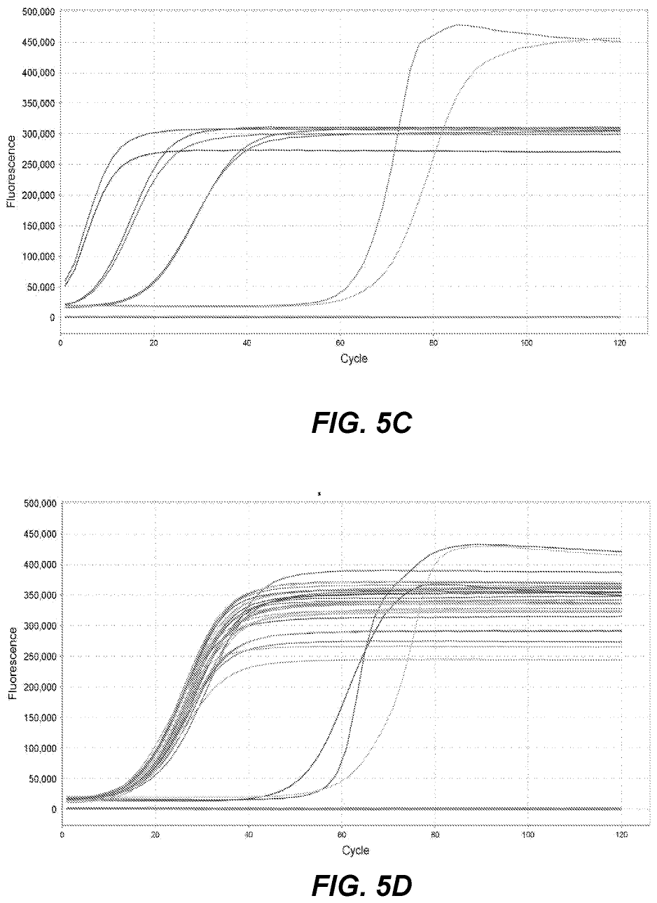

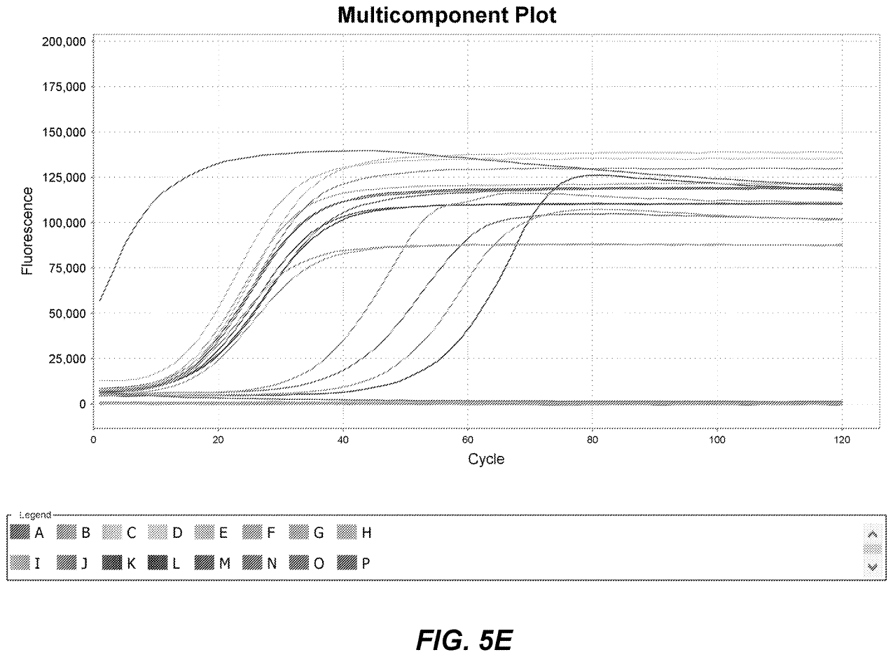

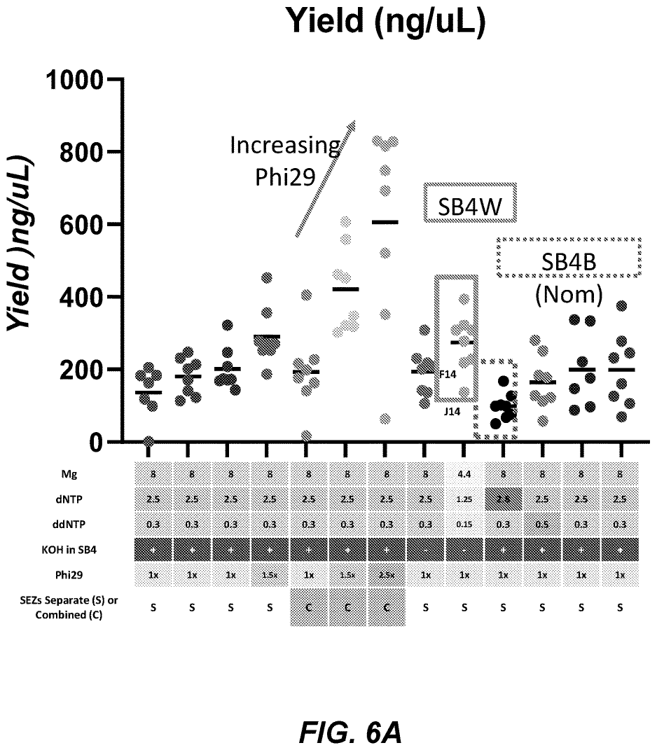

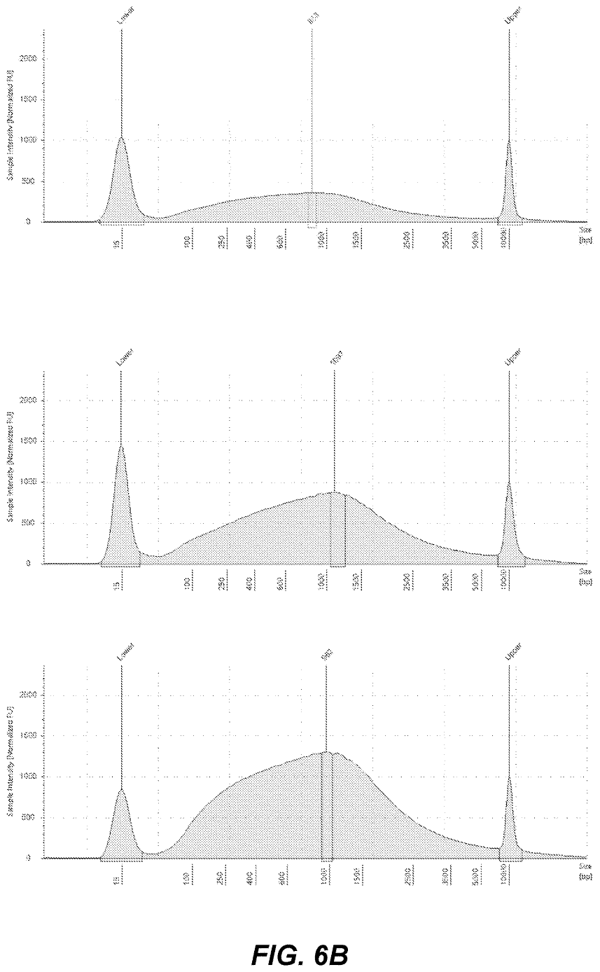

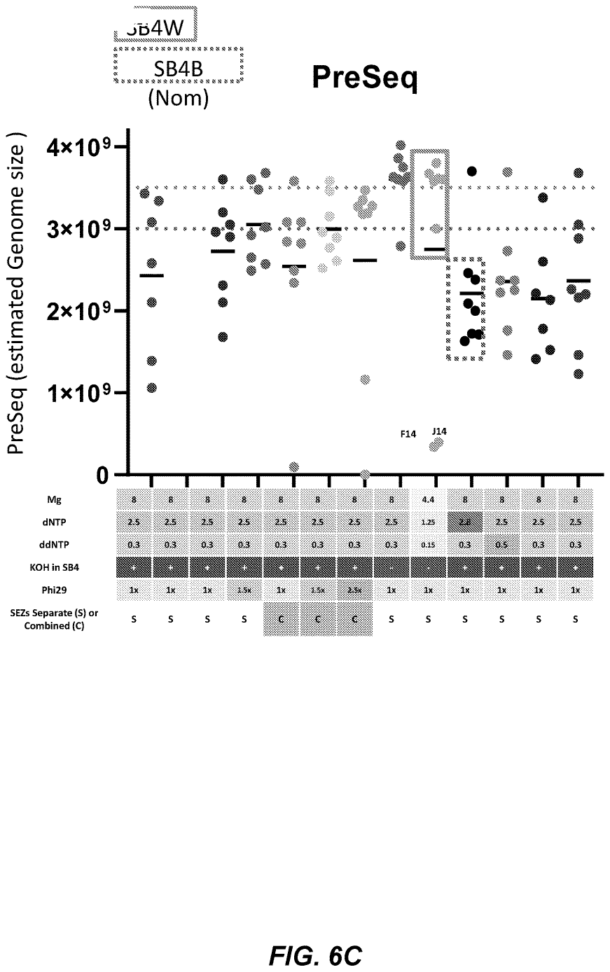

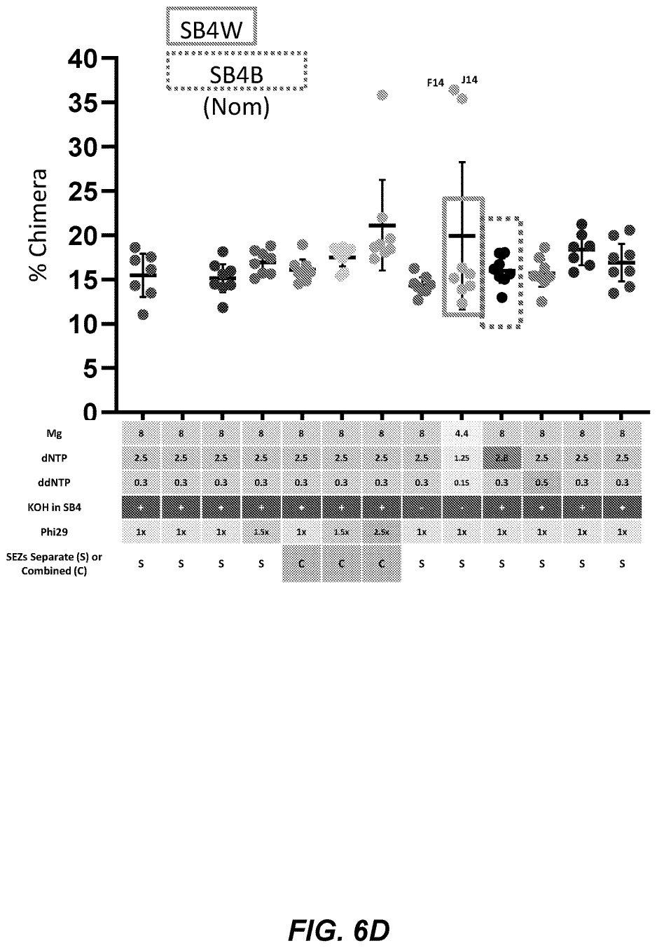

Real time PTA results for controls (SB4B and SB4W) are shown in FIGS. 5A and 5C, respectively and single cells (SB4B and SB4W) are shown in FIGS. 5B and 5D, respectively. Eight additional samples were also sequenced from FIG. 5E. Uncleaned PTA measured at 1:10 dilution by Qubit Flex. The yield was driven strongly by Phi29 concentration/amount in reaction, although no difference in yield was observed when combining SEZ1 and SEZ2. Despite the early amplification by C, yield for SB4W wasn't out of range. Late amplifying samples generally had a lower yield (greater separation in this if reaction time is reduced as well). Uncleaned PTA reactions were also put on a tapestation at a 1:100 dilution (~1-8 ng/μL). SB4W fragment size was not substantially different from other PTA formulations measured. (FIG. 6B).

Library preparation was also performed on samples obtained using these conditions. Library preparation was performed using Tecan d300/HP d100, 1 μL of 1:10 dilution of PTA reaction diluted into 3 μL Elution Buffer, Added 2 μL ERAT mix without enhancer (H₂O+0.4% Tween supplemented,

ERAT incubation, adding 1 μL Library Adapters from adapter plate, adding 1 μL Ligation Mix at 2× typical concentration, and performing ligation incubation. Samples were then multiplexed (8 samples) into 1 with 36 μL of 36 mM EDTA. Bead cleanup was performed, the library was amplified, and double sided bead cleanup was used. Sequencing results (pre-seq and chimera rates) are shown in FIGS. 6C-6D, respectively. SB4W conditions yielded highly diverse libraries, and increased enzyme also increases sequence diversity. Other samples performed less well than previously observed, but sequencing failures were predicted by real time PTA (F14, J14). Good quality SB4W samples were among the lower chimeric rate samples, and without increased SEZ1 was correlated with increased chimerism. Without being bound by theory, some chimerism may relate to library prep process.

### Example 4:2-Step PTA Reaction

A two-step PTA reaction was conducted according to the general method in FIG. 1A and Examples 1-3. Briefly, cells are isolated, treated with a lysis buffer/primer solution (“MS Mix”), incubated at room temperature, treated with a neutralization/PTA mix (“Reaction Mix”), incubated at 30° C. for 2.5 hours, and then converted to a sequencing ready library for analysis. Compared to the four step method, lysis and priming steps were combined, and neutralization and PTA reaction steps were combined (FIG. 1B).

Components: Components used in this example included cell buffer (1000 μL), SM3 reagent (500 μL), 12×SS2 reagent (500 μL), SDXT reagent (360 μL), SB5 reagent (1200 μL), SEZC reagent (100 μL), DNA/nuclease free water (500 μL), and control genomic DNA (50 ng/μL, 10 μL). Reagents were stored at −20° C. prior to use. The general function and components of the reagents are shown in Table 3A.

TABLE 3A

| Mix | Reagent | Step | Function | Components |
|---|---|---|---|---|
| MS Mix | SM3 Reagent | 1 | Cell lysis and | KOH, EDTA, (optionally HEPES) |
| | | | genome denaturation | |
| | 12X SS2 | 1 | Reducing agent | DTT |
| | Reagent | | | |
| | SDXT | 1 | Priming | Random hexamers |
| | Reagent | | | |
| Reaction Mix | SB5 Reagent | 2 | Reaction substrates | dNTPs, alpha-thio ddNTPs, salts, |
| | | | | HEPES, Tween, DTT, dye |
| | | | | (See Table 3B, formula SB5.1) |
| | SEZC Reagent | 2 | Enzyme mix | Phi29 Polymerase (99 units/μL), |
| | | | | SSB (single-strand DNA binding |
| | | | | protein, 6.15 ng/μL) |

SB5 and variant formulations thereof were also evaluated. These are described in Table 3B (concentrations in mM, volumes in microliters).

TABLE 3B

| | | Formulation | | | | | | | | | | |
|---|---|---|---|---|---|---|---|---|---|---|---|---|
| | | 2SHT | 2SHT | 4333 | 4333 | 4333-5.1 | 4333 | 4333 | 4333 | 4333 | 4333 | 4333 |
| | | Sub-Formulation | | | | | | | | | | |
| | | | SB3 | | SB5 | | | | | | | |
| | | (SB3) | actual | (SB5) | actual | (SB5.1) | | | | | | |
| | | L1 | L2 | L2 | L2 | L3, L4, L5 | (SB5.12) | SB5F | SB5G | Mid | High | Highest |
| Reagent | Component | | | | | | | | | | | |
| SM3 | mM KOH | 400 | 400 | 450 | 450 | 500 | 500 | 500 | 500 | 500 | 500 | 500 |
| | mM EDTA | 2 | 2 | 2.5 | 2.5 | 2.5 | 2.5 | 2.5 | 2.5 | 2.5 | 2.5 | 2.5 |
| | mM HEPES | 0 | 10 | 250 | 0 | 10 | 10 | 10 | 20 | 10 | 10 | 10 |
| SB5 | mM HEPES | 186.25 | 186.25 | 250 | 250 | 280 | 305 | 275 | 264 | 280 | 280 | 280 |
| | mM (NH₃)₂SO₄ | 16.67 | 16.67 | 22 | 22 | 25 | 27.3 | 24 | 23.2 | 25 | 25 | 25 |
| | mM MgCl₂ | 4.4 | 4.4 | 6.2 | 6.2 | 6.5 | 7.4 | 6.7 | 6.55 | 6.5 | 6.5 | 6.5 |
| | % Tween | 0.35 | 0.35 | 0.35 | 0.35 | 0.35 | 0.35 | 0.35 | 0.35 | 0.35 | 0.35 | 0.35 |
| | mM DTT | 6.2 | 6.2 | 6.2 | 6.2 | 7 | 7.5 | 6.75 | 6.53 | 7 | 7 | 7 |
| | X EvaGreen | 0.076 | 0.076 | 0.076 | 0.076 | 0.08 | 0.09 | 0.08 | 0.08 | 0.08 | 0.08 | 0.08 |
| | mM dATP | 1.25 | 1.25 | 1.2 | 1.2 | 1.35 | 1.5 | 1.32 | 1.27 | 1.35 | 1.35 | 1.35 |
| | mM dTTP | 1.25 | 1.25 | 1.2 | 1.2 | 1.35 | 1.5 | 1.32 | 1.27 | 1.35 | 1.35 | 1.35 |
| | mM dCTP | 1.25 | 1.25 | 1.2 | 1.2 | 1.35 | 1.5 | 1.32 | 1.27 | 1.35 | 1.35 | 1.35 |
| | mM dGTP | 1.25 | 1.25 | 1.2 | 1.2 | 1.35 | 1.5 | 1.32 | 1.27 | 1.35 | 1.35 | 1.35 |
| | mM ddATP | 0.15 | 0.15 | 0.2 | 0.2 | 0.24 | 0.265 | 0.22 | 0.21 | 0.24 | 0.24 | 0.24 |
| | mM ddCTP | 0.15 | 0.15 | 0.2 | 0.2 | 0.24 | 0.265 | 0.22 | 0.21 | 0.24 | 0.24 | 0.24 |
| | mM ddTTP | 0.15 | 0.15 | 0.2 | 0.2 | 0.24 | 0.265 | 0.22 | 0.21 | 0.24 | 0.24 | 0.24 |
| | mM ddGTP | 0.15 | 0.15 | 0.2 | 0.2 | 0.24 | 0.265 | 0.22 | 0.21 | 0.24 | 0.24 | 0.24 |
| Volumes | Lysis Vol | 1 | 1 | 2 | 2 | 2 | 2 | 2 | 2 | 2 | 2 | 2 |
| (micro | Rxn Vol | 3 | 3.2 | 4 | 4.2 | 4 | 4 | 4 | 4 | 4 | 4 | 4 |
| liters) | Vol Cell buffer | 0.2857 | 0.2857 | 1 | 1 | 1 | 1 | 1 | 1 | 1 | 1 | 1 |
| | in Lysis | | | | | | | | | | | |
| | Vol SM3 in Lysis | 0.2679 | 0.2679 | 0.37 | 0.37 | 0.375 | 0.375 | 0.375 | 0.375 | 0.4 | 0.44 | 0.48 |
| | Vol 12XSS2 | 0.0178 | 0.0178 | 0.03 | 0.03 | 0.025 | 0.025 | 0.025 | 0.025 | 0.04 | 0.04 | 0.04 |
| | Vol SB5 in Rxn | 1.85 | 2.035 | 1.8 | 1.98 | 1.85 | 1.85 | 1.85 | 1.85 | 1.8 | 1.8 | 1.8 |
| MS | Lysis [KOH] | 107.16 | 107.16 | 83.25 | 83.25 | 93.75 | 93.75 | 93.75 | 93.75 | 100.00 | 110.00 | 120.00 |
| Mix | Lysis [EDTA] | 0.54 | 0.54 | 0.46 | 0.46 | 0.47 | 0.47 | 0.47 | 0.47 | 0.50 | 0.55 | 0.60 |
| (mM) | Lysis [NaCl] | 39.14 | 39.14 | 68.50 | 68.50 | 68.50 | 68.50 | 68.50 | 68.50 | 68.50 | 68.50 | 68.50 |
| | Lysis [DTT] | 17.80 | 17.80 | 15.00 | 15.00 | 12.50 | 12.50 | 12.50 | 12.50 | 20.00 | 20.00 | 20.00 |
| | Lysis [Primers] | 2.86 | 2.86 | 5.00 | 5.00 | 5.00 | 5.00 | 5.00 | 5.00 | 5.00 | 5.00 | 5.00 |
| Full | [Cell Buffer] | 0.10 | 0.09 | 0.25 | 0.24 | 0.25 | 0.25 | 0.25 | 0.25 | 0.25 | 0.25 | 0.25 |
| Reaction | [NaCl] | 13.05 | 12.23 | 34.25 | 32.62 | 34.25 | 34.25 | 34.25 | 34.25 | 34.25 | 34.25 | 34.25 |
| | [KOH] | 35.72 | 33.49 | 41.63 | 39.64 | 46.88 | 46.88 | 46.88 | 46.88 | 50.00 | 55.00 | 60.00 |
| | [EDTA] | 0.18 | 0.17 | 0.23 | 0.22 | 0.23 | 0.23 | 0.23 | 0.23 | 0.25 | 0.28 | 0.30 |
| | mM HEPES | 114.85 | 118.44 | 112.50 | 117.86 | 129.50 | 141.06 | 127.19 | 122.10 | 126.00 | 126.00 | 126.00 |
| | mM (NH₃)₂SO₄ | 10.28 | 10.60 | 9.90 | 10.37 | 11.56 | 12.63 | 11.10 | 10.73 | 11.25 | 11.25 | 11.25 |
| | mM MgCl₂ | 2.71 | 2.80 | 2.79 | 2.92 | 3.01 | 3.42 | 3.10 | 3.03 | 2.93 | 2.93 | 2.93 |
| | % Tween | 0.22 | 0.22 | 0.16 | 0.17 | 0.16 | 0.16 | 0.16 | 0.16 | 0.16 | 0.16 | 0.16 |
| | mM DTT | 12.72 | 12.84 | 10.29 | 10.42 | 9.49 | 9.72 | 9.37 | 9.27 | 13.15 | 13.15 | 13.15 |
| | X EvaGreen | 0.05 | 0.05 | 0.03 | 0.04 | 0.04 | 0.04 | 0.04 | 0.04 | 0.04 | 0.04 | 0.04 |
| | mM dATP | 0.77 | 0.79 | 0.54 | 0.57 | 0.62 | 0.69 | 0.61 | 0.59 | 0.61 | 0.61 | 0.61 |
| | mM dTTP | 0.77 | 0.79 | 0.54 | 0.57 | 0.62 | 0.69 | 0.61 | 0.59 | 0.61 | 0.61 | 0.61 |
| | mM dCTP | 0.77 | 0.79 | 0.54 | 0.57 | 0.62 | 0.69 | 0.61 | 0.59 | 0.61 | 0.61 | 0.61 |
| | mM dGTP | 0.77 | 0.79 | 0.54 | 0.57 | 0.62 | 0.69 | 0.61 | 0.59 | 0.61 | 0.61 | 0.61 |
| | mM ddATP | 0.09 | 0.10 | 0.09 | 0.09 | 0.11 | 0.12 | 0.10 | 0.10 | 0.11 | 0.11 | 0.11 |
| | mM ddCTP | 0.09 | 0.10 | 0.09 | 0.09 | 0.11 | 0.12 | 0.10 | 0.10 | 0.11 | 0.11 | 0.11 |
| | mM ddTTP | 0.09 | 0.10 | 0.09 | 0.09 | 0.11 | 0.12 | 0.10 | 0.10 | 0.11 | 0.11 | 0.11 |
| | mM ddGTP | 0.09 | 0.10 | 0.09 | 0.09 | 0.11 | 0.12 | 0.10 | 0.10 | 0.11 | 0.11 | 0.11 |
| | Ratio Mg/NTPs | 0.79 | 0.79 | 1.11 | 1.11 | 1.02 | 1.05 | 1.09 | 1.11 | 1.02 | 1.02 | 1.02 |
| | Total NTPs | 3.45 | 3.56 | 2.52 | 2.64 | 2.94 | 3.27 | 2.85 | 2.74 | 2.86 | 2.86 | 2.86 |

Controls. The following controls in Table 4 were used:

TABLE 4

| Purpose | Formulation |
|---|---|
| No Template Control (NTC) | Cell Buffer Alone |
| High Input Positive Control | 1 ng gDNA |
| Mid Input Positive Control | 100 pg gDNA |
| Low Input Positive Control (Roughly | 10 pg gDNA |
| Equivalent to 1-1.5 Cells) | |

Each control was run in duplicate to baseline each experiment. The NTC helped detect contamination such as carryover from adjacent wells or the lab environment, which is helpful due to the high sensitivity of PTA to ultra-low levels of nucleic acid in a sample. Bulk gDNA controls help assess the correct execution of the protocol and quantitative accuracy.

Manual Pipetting Technique: To avoid material loss in the reaction, direct contact between pipet tips and the cell suspension, lysate, or other reaction intermediaries during manual reagent additions was avoided. Loss of a small amount of liquid is unavoidable whenever the pipet tip is allowed to come into contact with the reaction mix. All reagent additions were dispensed onto the wall of the tube.

Gentle and Thorough Mixing: Once the reagent was added to the tube, gentle and thorough mixing of the reaction components was employed. Non-homogeneity within the reaction may lead to inefficiency and diminish performance. To ensure each reagent addition is mixed into the reaction thoroughly, plate/tubes were sealed and briefly spun in a centrifuge/plate spinner (10 seconds at ~750×g was sufficient). Just enough force was used to combine the added droplet with the material in the bottom of the tube.

Once the added droplet was combined with the reaction components in the bottom of the tube, the reaction plate/tubes was placed in a programmable thermal mixer and gently mixed. After mixing, reactions were spun again to ensure any droplets generated during the mixing process were recombined in the bottom of the plate/tubes.

Pipet reagent additions were made on the side of the tube, avoiding any contact with the material in the bottom of the tube, then SEAL-SPIN-MIX-SPIN. After these steps, incubation or the next reagent addition was carried out.

Quantification: A fluorometric method of quantification (such as Qubit) with the amplification products and sequencing libraries was used.

The protocol was optimized for single live mammalian cells, and correspondingly, ultra-low amounts of DNA input (4 pg-10 ng). Input can be either single or multiple cells, obtained by common cell collection methods including fluorescence-activated cell sorting (FACS), fluorescence-activated nuclei sorting (FANS), laser capture microdissection (LCM), and other cell capture techniques. No upper limit has been established for multiple cell input, but theoretically dozens to hundreds of cells per reaction are feasible. Viable single cells were placed into 1 μL of Cell Buffer, then subjected to the PTA method or frozen at −80° C. for short-term storage.

### Experiment Preparation

This example allowed processing of single or multiple cells (or nuclei) through a PTA-mediated genome amplification process. Reagent additions were optimally carried out using an automated liquid handler to facilitate a throughput of up to 384 samples per run. The genome amplification took place in an isothermal incubation lasting 2.5 hours which is carried out in a thermal cycler. Cells were placed into the wells of a 384-well plate containing 1 μL of Cell Buffer directly via FACS/FANS or other methods. Cells may also be sorted “dry” into empty wells if desired. In cases where cells are “dry” sorted, 1 μL of Cell Buffer was added to each well prior to beginning the protocol. If wells contained less than 1 μL, Cell Buffer was added to bring the volume to 1 μL.

The example protocol was carried out in a DNA-free, pre-amplification workspace or PCR hood enclosure to avoid the possible introduction of exogenous DNA from the operator or the lab environment. A no-template control (NTC) was further used to identify an extraneous sources of nucleic acids. Positive control reactions were run at a range of input concentrations. Traditional vortex mixers on cells, lysates, and other reaction intermediaries during the protocol can lead to poor performance. Vortexing can result in uneven mixing of reactions, leading to variable performance and splashing of tube contents on the plate seal or tube lid, resulting in loss of genome coverage. A vortex mixer was used to thoroughly mix all reagents after thawing except SEZC Reagent. All reactions and reagents were kept on ice unless stated otherwise. When referencing “briefly spin down,” the intent was to ensure any droplets dispersed within a tube are collected. A quick pulse (3-5 seconds) on a benchtop microcentrifuge was usually sufficient.

### Protocol I: Reagent Retrieval and Control Setup

Procedure: Components were retrieved from −20° C. storage. 12×SS2, Control gDNA, DNA/Nuclease-free Water, and Cell Buffer were placed at room temperature to thaw. SM3, SDXT, and SB5 were placed on ice to thaw for 30 minutes to 1 hour. SEZC was left in −20° C. storage until needed. Once the reagents thawed, they were vortexed for 5 seconds, briefly spun down, and placed on ice. Important: Once 12×SS2 was thawed, it was vortexed thoroughly until any precipitate was fully dissolved, briefly spun down, and placed on ice.

Control Setup: A 1 ng/μL gDNA stock was prepared by adding 1 μL of Control gDNA to 49 μL of Cell Buffer in a labeled microcentrifuge tube. The 1 ng/μL gDNA was vortexed for 5 seconds, briefly spin down, and placed on ice. Optionally, the 1 ng/μL Control gDNA stock was verified to be the intended concentration using a Qubit fluorometer. Note: If the concentration deviates from the expected concentration Ing/μL by more than 10%, the dilution factor in subsequent dilutions was modified to achieve the desired 100 μg/μL and 10 pg/μL concentrations. Two additional microcentrifuge tubes were set up and label them 100 pg/μL, and 10 μg/μL. The 1 ng/μL gDNA prepared above was serially diluted according to Table 5. The 100 μg/μL solution was prepared by taking 2 μL from the 1 ng/μL gDNA solution and adding 18 μL of Cell Buffer. Similarly, 2 μL from the 100 μg/μL solution was used and 18 μL of Cell Buffer was added to create the 10 μg/μL solution. After each dilution, vortexing occurred thoroughly for 5 seconds, the solutions briefly spun down, and placed on ice before proceeding to the next dilution step.

TABLE 5
Control Serial Dilution Setup

| Tube Label | gDNA | Cell Buffer |
|---|---|---|
| 1 ng/μL gDNA | 1 μL of Control gDNA | 49 μL |
| 100 pg/μL gDNA | 2 μL of 1 ng/μL gDNA | 18 μL |
| 10 pg/μL gDNA | 2 μL of 100 pg/μL gDNA | 18 μL |

A thermal cycler was programmed with a 384-well block installed to run the DNA amplification program (Table 6).

TABLE 6
DNA Amplification (lid temperature 105°
C., reaction volume 4 μL)

| Step | Temperature | Time | |
|---|---|---|---|
| Hold 1 | 30° C. | 2.5 | hours |
| Hold 2 | 65° C. | 3 | minutes |
| Hold 3 | 4° C. | ∞ | |
| Total Time | — | ~2.6 | hours |

The plate containing samples was placed on ice. If cells were stored at −80° C., cells were thawed on ice for 5 minutes, spun for 10 seconds, and placed on ice. If cells were fresh they were maintained on ice and amplification steps proceeded promptly. If cells were suspended in less than 1 μL, Cell Buffer was added to bring them up to 1 μL.

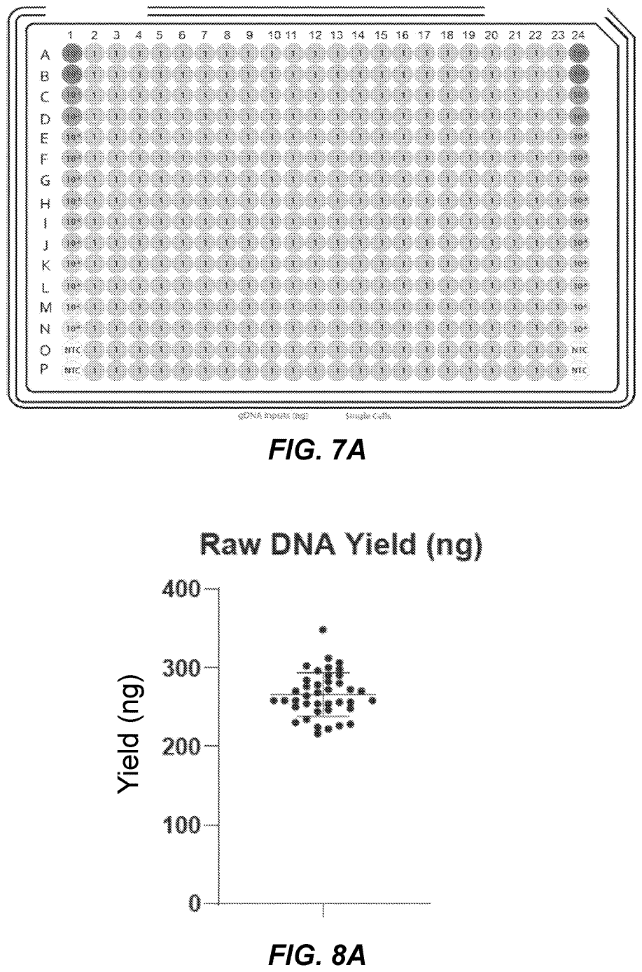

Dispense DNA controls. 1 μL of Cell Buffer was added to each Negative Control (NTC) well. 1 μL of 10 μg/μL gDNA dilution was added to each 10 μg Control gDNA well. 1 μL of 100 μg/μL gDNA dilution was added to each 100 μg Control gDNA well. 1 μL of 1 ng/μL gDNA dilution was added to each 1 ng Control gDNA well. The plate layout is shown in FIG. 7.

For “Step 1” of the 2-step PTA process, MS Mix was prepared by combining the following reagents in a microcentrifuge well (Table 7).

TABLE 7
Volume of Components in MS Mix for
Reactions in a 384 Well Plate

| | | | Volume per |
|---|---|---|---|
| | Volume per | Volume per | 384 Reactions |
| Component | Reaction (μL) | 96 Reactions (μL)* | (μL)* |
| SDXT Reagent | 0.600 | 74.9 | 299.5 |
| 12X SS2 Reagent | 0.025 | 3.1 | 12.5 |
| SM3 Reagent | 0.375 | 46.8 | 187.2 |
| Total Volume | 1.0 | 124.8 | 499.2 |

*30% overage included

After mixing, the tubes were vortexed 5 seconds to mix, briefly spun down, and placed on ice. Using an automated liquid handler, 1 μL of MS Mix was added to each well. The plate was sealed and spun down for ~10 sec to combine components. In a thermal mixer, mixing was conducted at room temperature for 20 min at 1,400 rpm. During incubation the following steps were completed: The DNA amplification protocol was started (see Table 6) on the thermal cycler and the block allowed to reach the incubation set point of 30° C. The thermal cycler was then paused. The Reaction Mix was then prepared by combining the components in the order listed in Table 8 on ice.

TABLE 8
Volume of Components in Reaction Mix

| | | Volume per | Volume per |
|---|---|---|---|
| | Volume per | 96 Reactions | 384 Reactions |
| Component | Reaction (μL) | (μL)* | (μL)* |
| SB5 Reagent | 1.85 | 284.2 | 1136.6 |
| SEZC Reagent | 0.15 | 23.0 | 92.2 |
| Total Volume | 2.0 | 307.2 | 1228.8 |

*60% overage included

For “Step 2” of the 2-step PTA process, the Reaction Mix was pipetted up and down 10 times with the pipet set to 50% of the total volume to mix, briefly spun down, and placed on ice. Care was taken to avoid creating air bubbles when pipet mixing. The plate was removed from thermal mixer, spun down for 10 seconds, and placed on ice. Using an automated liquid handler, 2 μL of the Reaction Mix was added to each well. The plate was then sealed and spun down for 10 seconds, mixed at room temperature in the thermal mixer for 1 min at 1000 rpm, spun down for 10 seconds, and placed on ice. The plate was kept on ice until the thermal cycler had reached 30° C., then the plate was loaded and the thermal cycler program unpaused. After the program was complete, the plate was removed, spun down for 10 seconds, and placed on ice. The Quality Control procedure described below was then conducted, or samples stored overnight at −20° C.

### III. Protocol III: Quality Control Checkpoint

A DNA quantification and fragment size distribution analysis was conducted on the PTA amplification products. Purification of the amplified DNA was not required if using the 384 reaction, semi-automated protocol to run 4 μL reactions.

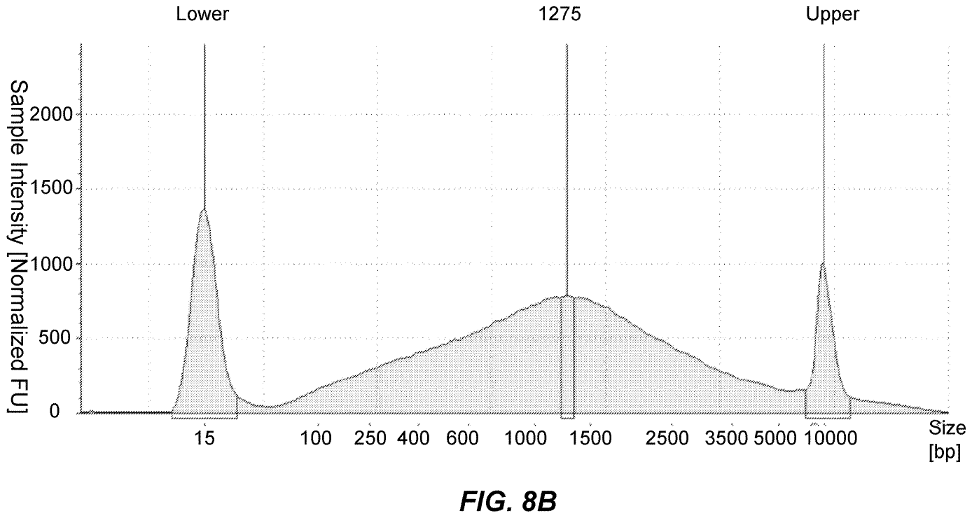

Procedure: 1. To assess DNA yield, each reaction was diluted 5-fold by adding 16 μL of Elution Buffer. 2 μL of diluted reaction mix was then added to 198 μL Qubit reagent and the concentration measured per manufacturer's instructions. Average DNA amplification yields were around 260 ng from single human cells, with a range between 220 ng and 350 ng (FIG. 8A). A 2 ng/μL dilution in a fresh PCR plate was prepared a by pipetting amplified DNA samples into Elution Buffer, the plate sealed, vortexed briefly, and spun down. Fragment size distribution was determined by running 2 μL of each 2 ng/μL diluted sample using a Tapestation HS D5000 Screentape or other fragment analysis instrument using manufacturer's instructions. Next, sequencing library preparation was conducted. An electropherogram representing the amplified sample which has been normalized to 2 ng/μL and run on a Tapestation using the D5000 HS Screentape is shown in FIG. 8B. Average fragment size was 1275 bp.

### Sequencing Library Preparation

A ResolveDNA® Preparation Kit (Bioskryb Genomics) was used to prepare next generation sequencing libraries from the amplified DNA produced in preparation for next generation sequencing. The library preparation process added sequencing adapters and barcodes for multiplex sequencing on Illumina® sequencing platforms.

### Initial Low-Pass Sequencing

An initial round of low depth sequencing was performed (2-5 million reads per cell) to check library complexity and ensure the single-cell libraries were uniformly amplified as desired, as well as to estimate data quality prior to performing high-depth sequencing. Typically, samples sequenced to this level were suitable for use in copy number variation (CNV) analysis. Generally, suitable data included up to 5 million reads/cell for CNV analysis. For single nucleotide variant analysis (SNV) and analysis of other genomic structural variation (such as detection of fusions and indels), deep sequencing (25-30× genomic coverage) was used.

### Data Analysis for Sample QC and Triage for Deep Sequencing

The BioSkryb BaseJumper® Bioinformatics platform contains an analytical pipeline, PreSeq, which utilizes algorithms designed to evaluate low depth sequencing data to predict genome coverage at higher depth sequencing levels and for visualizing other general performance metrics. The following pipeline capabilities were used to analyze sequencing data:

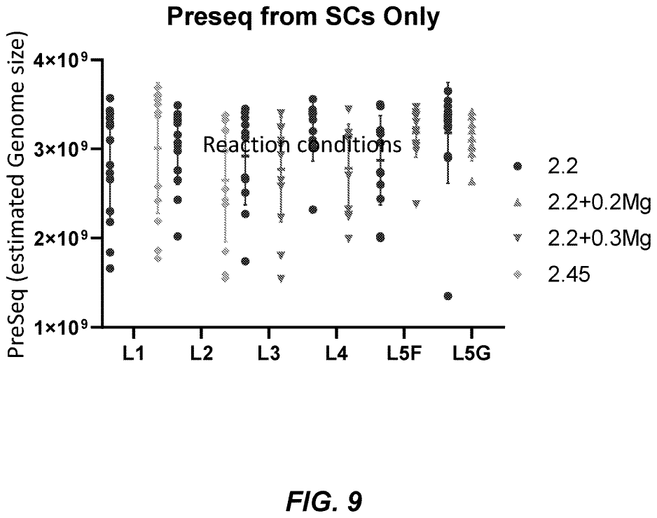

PreSeq (Quality Control): Library complexity, error rates, chromosomal coverage, library anomalies, and read counts are among the metrics returned. This quickly determined the quality of sequencing data (by down sampling) before moving on to longer, more expensive analyses. Low pass data for various formulations of SB5 is shown in FIG. 9. The circle series (Table 3A, 4333-5.1) between L3 and L4 in FIG. 9 provided high genome size (indicative of sequencing quality) and high consistency among cells tested.

WGS pipeline: The genomic pipeline analyzed single nucleotide variants (SNVs), small insertions or deletions (Indels), and copy number variants (CNVs), (data not shown). Exome or other targeted panel capture methods can be used to contextualize these data against regions of interest.

### Example 5: Comparison of 2-Step and 4-Step Methods

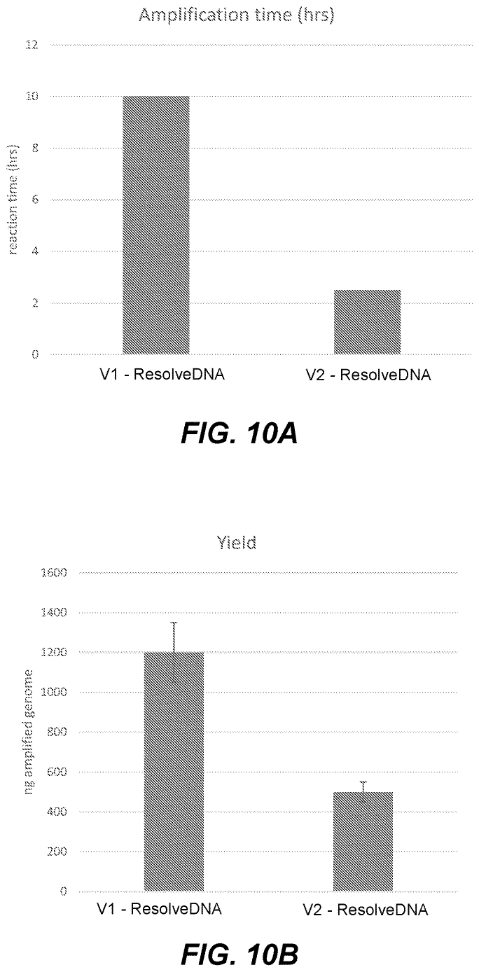

A two-step PTA reaction was conducted according to the general method in FIG. 1A to demonstrate several advantages. Briefly, cells are isolated, treated with a lysis buffer/primer solution (“MS Mix”), incubated at room temperature, treated with a neutralization/PTA mix (“Reaction Mix”), incubated at 30° C. for 2.5 hours, and then converted to a sequencing ready library for analysis (4333-5.1). Compared to the four step method, lysis and priming steps were combined, and neutralization and PTA reaction steps were combined (FIG. 1B). These reactions were then compared to previous legacy versions of PTA chemistry. First an improvement in time savings of the 2-step reaction was observed. The time required to generate sufficient amplified genome material was reduced approximately 4× (FIG. 10A). The material required for effective library preparation (~100 ng) was produced within the amplification time for both reactions. The reaction time was further reduced by lowering the quantity of detergent in the reaction, to roughly 0.5-1.5 hrs based on the data from real-time monitoring of the reaction.

Sequencing Library Preparation Amplified material weas then converted through a ligation and amplification process to create dual indexed short read sequencing libraries.

A ResolveDNA® Preparation Kit (Bioskryb Genomics) was used to prepare next generation sequencing libraries from the amplified DNA produced in preparation for next generation sequencing. The library preparation process added sequencing adapters and barcodes for multiplex sequencing on Illumina® sequencing platforms.

The examples described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

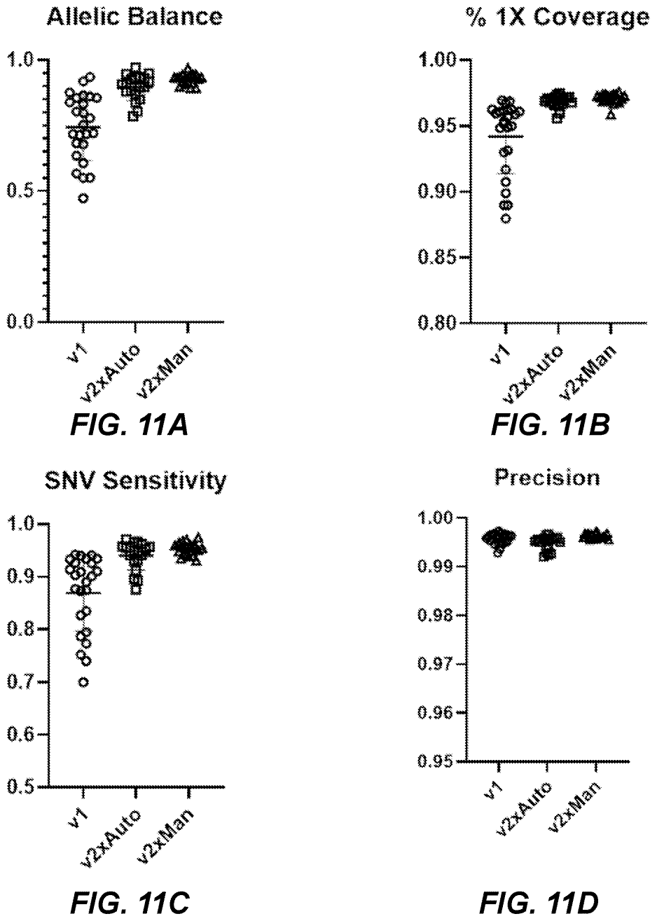

Data quality was a significant improvement over the legacy PTA (V1 ResolveDNA) presented in the adjacent figure (FIGS. 11A-11D). This was evident in the improvement of Allelic balance, coverage, and sensitivity. While the precision was unchanged this is also a significant result as typically what is observed with increases in SNV sensitivity is a coordinated decrease in precision.

This chemistry was then tested against a series of specifications to determine the statistical confidence (Table 9).

TABLE 9

| Performance | | | Passing | Observed |
|---|---|---|---|---|
| Characteristic | Analyte | Analytical Description | Criteria | Value |
| Accuracy | Single Cell DNA | Concordance of PTA amplified single DNA single | >99% | 99.99% |
| | | nucleotide variants (SNVs) with NIST validated | | |
| | | reference sequence | | |
| Sensitivity | Single Cell DNA | A measure of true SNVs called relative to all | >90% | 95.37% |
| | | variants | | |
| Specificity | Single Cell DNA | A measure of non-SNV variants not called as SNV | >90% | 99.99% |
| | | variants relative to total non-variants based on | | |
| | | HG001 reference standard | | |
| Precision | Single Cell DNA | Concordance of PTA amplified single DNA SNVs | >99% | 99.63% |
| | | with other cells from the same sample | | |
| Allelic Balance | Single Cell DNA | Detection of both alleles in regions of known | >85% | 92.7% |
| | | heterozygosity in the NIST validated reference | | |
| | | sequence | | |
| Coverage | Single Cell DNA | A measure of the degree of unevenness in the | <25% | 19.7% |
| Uniformity | | sequencing depth distribution across the genome | | |
| | | utilizing the Gini Coefficient | | |
| Genome | Single Cell DNA | The percentage of the reference genome that is | >90% | 97.1% |
| Completeness | | covered by at least one read | | |
| Failure Rate | Single Cell DNA | Percentage of cells with below specification DNA | <20% | 2.8% |
| | | yield | | |
| Limit of Detection | Genome | N/A; Assay is performed at the lower limit of a | N/A | N/A |
| | | genome (1 cell) | | |

Detailed analysis of deep sequencing single cells demonstrated the high reproducibility of the individual reactions carried out by various operators. The data demonstrated superior performance metrics in terms of genome coverage, sensitivity, precision, accuracy and specificity, which was driven by high allelic balance and a low Gini coefficient.

TABLE 10A

| | | | | | | PF Reads | Mean | PCT | PCT |
|---|---|---|---|---|---|---|---|---|---|
| Biosample: Same | Lot | Kit | Cell | Operator | PF Reads | Aligned | Coverage | 1X | 5X |
| BSB09405-3117-C-4A-D-02f | 8a | 1 | SC14 | A | 765775584 | 763686552 | 30.337 | 0.973 | 0.936 |
| BSB09405-3117-C-4A-D-02G | 8a | 1 | SC15 | A | 1238293769 | 1235149146 | 47.133 | 0.976 | 0.950 |
| BSB09405-3117-E-4B-D-05E | 8a | 1 | SC15 | B | 974265307 | 972182108 | 37.153 | 0.959 | 0.926 |
| BSB09405-3117-E-4B-D-05F | 8a | 1 | SC16 | B | 7562133572 | 750343549 | 29.817 | 0.968 | 0.917 |
| BSB09405-3117-C-5A-E-02G | 9a | 1 | SC15 | A | 824957472 | 823104725 | 30.050 | 0.971 | 0.926 |
| BSB09405-3117-C-5A-E-02H | 9a | 1 | SC16 | A | 1136252841 | 1133871613 | 41.758 | 0.974 | 0.941 |
| BSB09405-3117-E-5B-E-05C | 9a | 1 | SC12 | B | 803086209 | 801507814 | 30.740 | 0.973 | 0.934 |
| BSB09405-3117-E-5B-E-05D | 9a | 1 | SC13 | B | 1091587323 | 1089658729 | 42.331 | 0.974 | 0.946 |
| BSB09405-3117-C-1A-A-01A | 10 | 1 | SC01 | A | 793086583 | 791135087 | 28.875 | 0.970 | 0.921 |
| BSB09405-3117-C-1A-A-01B | 10 | 1 | SC02 | A | 744535467 | 742869578 | 27.536 | 0.970 | 0.919 |
| BSB09405-3117-E-1C-A-05A | 10 | 1 | SC10 | B | 513497067 | 511890655 | 20.056 | 0.969 | 0.904 |
| BSB09405-3117-E-1C-A-04H | 10 | 1 | SC9 | B | 424525317 | 423139391 | 17.277 | 0.967 | 0.886 |
| BSB09405-3117-C-2A-B-03f | 10 | 2 | SC22 | A | 911409943 | 908951521 | 34.759 | 0.974 | 0.938 |
| BSB09405-3117-C-2A-B-03G | 10 | 2 | SC23 | A | 841456461 | 839297237 | 32.962 | 0.970 | 0.930 |
| BSB09405-3117-B-2B-B-05G | 10 | 2 | SC14 | B | 818332686 | 816332994 | 31.858 | 0.971 | 0.927 |
| BSB09405-3117-B-2B-B-05H | 10 | 2 | SC15 | B | 910908088 | 908848835 | 34.858 | 0.974 | 0.937 |
| BSB09405-3117-C-3A-C-02E | 10 | 3 | SC13 | A | 845581503 | 843687056 | 31.466 | 0.972 | 0.928 |
| BSB09405-3117-C-3A-C-02f | 10 | 3 | SC14 | A | 861745192 | 859700663 | 31.684 | 0.969 | 0.924 |
| BSB09405-3117-E-3B-C-06C | 10 | 3 | SC19 | B | 870993392 | 869269000 | 32.717 | 0.973 | 0.935 |
| BSB09405-3117-E-3B-C-06D | 10 | 3 | SC20 | B | 864945678 | 863259850 | 31.857 | 0.973 | 0.934 |
| Single Cell Average | | | | | 849368473 | 847394305 | 32.261 | 0.971 | 0.928 |
| ±95% Confidence Interval | | | | | 80687063 | 80557398 | 2.976 | 0.002 | 0.006 |

TABLE 10B

| | | Proportion | | | | | | | |
|---|---|---|---|---|---|---|---|---|---|
| Biosample: | Proportion | Mito | | | F- | | | Allelic | Gini |
| Same | Chimeras | (chrM) | Precision | Sensitivity | measure | Specificity | Accuracy | Balance | Coefficient |
| BSB09405-3117- | 0.085 | 0.002 | 0.9968 | 0.9673 | 0.9818 | 0.999996 | 0.999955 | 0.943438 | 0.190304 |
| C-4A-D-02f | | | | | | | | | |
| BSB09405-3117- | 0.106 | 0.002 | 0.9968 | 0.9570 | 0.9765 | 0.999996 | 0.999943 | 0.914012 | 0.196822 |
| C-4A-D-02G | | | | | | | | | |
| BSB09405-3117- | 0.093 | 0.003 | 0.9964 | 0.9509 | 0.9731 | 0.999996 | 0.999934 | 0.945128 | 0.197243 |
| E-4B-D-05E | | | | | | | | | |
| BSB09405-3117- | 0.097 | 0.003 | 0.9955 | 0.9308 | 0.9621 | 0.999995 | 0.999908 | 0.891730 | 0.202054 |
| B-4B-D-05F | | | | | | | | | |
| BSB09405-3117- | 0.091 | 0.003 | 0.9962 | 0.9557 | 0.9756 | 0.999995 | 0.999940 | 0.931944 | 0.199788 |
| C-5A-E-02G | | | | | | | | | |
| BSB09405-3117- | 0.091 | 0.002 | 0.9968 | 0.9638 | 0.9800 | 0.999996 | 0.999951 | 0.933605 | 0.198975 |
| C-5A-E-02H | | | | | | | | | |
| BSB09405-3117- | 0.079 | 0.002 | 0.9970 | 0.9742 | 0.9855 | 0.999996 | 0.999964 | 0.968453 | 0.186121 |
| E-5B-E-05C | | | | | | | | | |
| BSB09405-3117- | 0.074 | 0.002 | 0.9972 | 0.9703 | 0.9835 | 0.999997 | 0.999959 | 0.944250 | 0.196797 |
| E-5B-E-05D | | | | | | | | | |
| BSB09405-3117- | 0.096 | 0.002 | 0.9957 | 0.9394 | 0.9667 | 0.999995 | 0.999919 | 0.907743 | 0.202179 |
| C-1A-A-01A | | | | | | | | | |
| BSB09405-3117- | 0.091 | 0.003 | 0.9960 | 0.9555 | 0.9753 | 0.999995 | 0.999940 | 0.933918 | 0.197968 |
| C-1A-A-01B | | | | | | | | | |
| BSB09405-3117- | 0.087 | 0.002 | 0.9960 | 0.9545 | 0.9748 | 0.999995 | 0.999938 | 0.943052 | 0.188418 |
| E-1C-A-05A | | | | | | | | | |
| BSB09405-3117- | 0.087 | 0.001 | 0.9955 | 0.9433 | 0.9687 | 0.999995 | 0.999924 | 0.931142 | 0.188699 |
| B-1C-A-04H | | | | | | | | | |
| BSB09405-3117- | 0.100 | 0.003 | 0.9965 | 0.9607 | 0.9783 | 0.999996 | 0.999947 | 0.935773 | 0.198171 |
| C-2A-B-03f | | | | | | | | | |
| BSB09405-3117- | 0.089 | 0.002 | 0.9966 | 0.9386 | 0.9667 | 0.999996 | 0.999919 | 0.891880 | 0.201748 |
| C-2A-B-03G | | | | | | | | | |
| BSB09405-3117- | 0.094 | 0.003 | 0.9960 | 0.9480 | 0.9714 | 0.999995 | 0.999930 | 0.916320 | 0.199063 |
| E-2B-B-05G | | | | | | | | | |
| BSB09405-3117- | 0.097 | 0.003 | 0.9963 | 0.9625 | 0.9791 | 0.999996 | 0.999949 | 0.937191 | 0.198559 |
| B-2B-B-05H | | | | | | | | | |
| BSB09405-3117- | 0.096 | 0.002 | 0.9962 | 0.9456 | 0.9703 | 0.999995 | 0.999928 | 0.912558 | 0.200202 |
| C-3A-C-02E | | | | | | | | | |
| BSB09405-3117- | 0.098 | 0.003 | 0.9960 | 0.9349 | 0.9645 | 0.999995 | 0.999914 | 0.898429 | 0.203475 |
| C-3A-C-02f | | | | | | | | | |
| BSB09405-3117- | 0.087 | 0.003 | 0.9958 | 0.9594 | 0.9773 | 0.999995 | 0.999944 | 0.927930 | 0.200124 |
| E-3B-C-06C | | | | | | | | | |
| BSB09405-3117- | 0.086 | 0.003 | 0.9989 | 0.9613 | 0.9783 | 0.999998 | 0.999947 | 0.932047 | 0.196984 |
| E-3B-C-06D | | | | | | | | | |
| Single Cell | 0.091 | 0.002 | 0.9963 | 0.9537 | 0.9745 | 0.999996 | 0.999938 | 0.927027 | 0.196885 |
| Average | | | | | | | | | |
| ±95% Confidence | 0.003 | 0.000 | 0.0002 | 0.0053 | 0.0028 | 0.000000 | 0.000007 | 0.008631 | 0.002168 |
| Interval | | | | | | | | | |

The examples described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of amplification from single cells comprising:
(a) contacting a single cell with lysis buffer, wherein the lysis buffer comprises at least one amplification primer;
(b) adding at the same time to the lysis buffer: a neutralization buffer, at least one nucleic acid polymerase, and a mixture of nucleotides, wherein the mixture of nucleotides comprises at least one terminator nucleotide which terminates nucleic acid replication by the polymerase; and
(c) amplifying at least some of the genome to generate a plurality of terminated amplification products, wherein the replication proceeds by strand displacement replication, wherein if the lysis buffer or neutralization buffer comprises TWEEN-20, the amount of TWEEN-20 is no more than 0.2% (v/v).

2. The method of claim 1, wherein the lysis buffer comprises one or more of a base, a buffering agent and a chelating agent.

3. The method of claim 2, wherein the buffering agent comprises HEPES (4-(2-hydroxyethyl)-1-piperazineethanesulfonic acid, BES, MOPS, TES, DIPSO, MOBS, TAPSO, Trizma, HEPPSO, POPSO, or TEA.

4. The method of claim 1, wherein the concentration of one or more components of the lysis buffer ranges from about 10 mM to about 500 mM.

5. The method of claim 1, wherein the pH of the lysis buffer is about 11 to about 14.

6. The method of claim 1, wherein the step (b) is performed for more than 10 minutes.

7. The method of claim 1, wherein the step (b), step (c), or both step (b) and step (c) is performed for less than 10 hours.

8. The method of claim 1, wherein the step (b), step (c), or both step (b) and step (c) is performed for less than 1 hour.

9. The method of claim 1, wherein the method further comprises addition of an ERAT and/or ligation mixture directly after the step (c).

10. The method of claim 1, wherein the terminator is an irreversible terminator.

11. The method of claim 10, wherein the terminator nucleotide is selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids.

12. The method of claim 10, wherein the nucleotides with modification to the alpha group are alpha-thio dideoxynucleotides.

13. The method of claim 1, wherein the terminator nucleotide comprises modifications of the r group of the 3′ carbon of the deoxyribose.

14. The method of claim 1, wherein the terminator nucleotide is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof.

15. The method of claim 1, wherein the polymerase has substantially no polymerase activity until heated to at least 40 degrees C.

16. The method of claim 1, wherein the concentration of magnesium is less than 8 mM.

17. The method of claim 1, wherein the mixture of nucleotides comprises at least one dNTP.

18. The method of claim 17, wherein the at least one dNTP is present at a concentration of 0.1-5 mM.

19. The method of claim 18, wherein the at least one dNTP is present at a concentration of less than 2.5 mM.

20. The method of claim 1, wherein the at least one terminator nucleotides is present at a concentration of less than 0.3 mM.

21. The method of claim 1, wherein the method further comprises sequencing the plurality of terminated amplification products or amplicons thereof.

22. The method of claim 21, wherein sequencing results in one or more of:
(a) an allelic balance of at least 0.8;
(b) a 1× coverage of at least 0.95;
(c) a precision of at least 0.99; and
(d) an SNV sensitivity of at least 0.85.

23. The method of claim 22, wherein the single cell is NA12878.

24. The method of claim 1, wherein the method comprises analysis of at least 100 cells.

25. The method of claim 1, wherein the method comprises analysis of at least 1000 cells.

26. A composition comprising at least one amplification primer, at least one nucleic acid polymerase, a buffer, and a mixture of nucleotides, wherein the mixture of nucleotides comprises at least one dNTP and at least one terminator nucleotide which terminates nucleic acid replication by the polymerase, where the buffer comprises magnesium, wherein concentration of magnesium is less than 8 mM.

27. The composition of claim 26, wherein the composition further comprises a target nucleic acid molecule.

28. The composition of claim 26, wherein magnesium concentration is 1-6 mM.

29. The composition of claim 26, wherein the magnesium concentration is 2-4 mM.

30. The composition of claim 26, wherein buffer comprises magnesium chloride.

31. The composition of claim 26, wherein the at least one dNTP is present at a concentration of 0.1-5 mM.

32. The composition of claim 26, wherein the at least one dNTP is present at a concentration of less than 2.5 mM.

33. The composition of claim 26, wherein the at least one dNTP is present at a concentration of 0.5-2 mM.

34. The composition of claim 26, wherein the at least one terminator nucleotide is present at a concentration of less than 0.3 mM.

35. The composition of claim 26, wherein the at least one terminator nucleotide is present at a concentration of 0.05-0.20 mM.

36. The composition of claim 26, wherein the concentration ratio of dNTPs and terminators to magnesium is 0.5-1.5.

37. The composition of claim 26, wherein the terminator is an irreversible terminator.

38. The composition of claim 37, wherein the terminator nucleotide is selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids.

39. The composition of claim 38, wherein the nucleotides with modification to the alpha group are alpha-thio dideoxynucleotides.

40. The composition of claim 26, wherein the terminator nucleotide comprises modifications of the r group of the 3′ carbon of the deoxyribose.

41. The composition of claim 26, wherein the terminator nucleotide is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof.

42. The composition of claim 26, wherein the composition further comprises a lysis buffer.

43. The composition of claim 42, wherein the lysis buffer comprises one or more of a base, a buffering agent and a chelating agent.

44. The composition of claim 43, wherein the buffering agent comprises HEPES (4-(2-hydroxyethyl)-1-piperazineethanesulfonic acid, BES, MOPS, TES, HEPES (4-(2-hydroxyethyl)-1-piperazineethanesulfonic acid), DIPSO, MOBS, TAPSO, Trizma, HEPPSO, POPSO, or TEA.

45. The composition of claim 42, wherein the concentration of one or more components of the lysis buffer ranges from about 10 mM to about 500 mM.
